(19) United States
(12) Patent Application Publication
(10) Pub. No.: US 2024/0141291 A1
(43) Pub. Date: May 2, 2024

(54) FUNCTIONAL BRAIN REGION-SPECIFIC NEURAL SPHEROIDS AND METHODS OF USE

(71) Applicant: THE UNITED STATES OF AMERICA, AS REPRESENTED BY THE SECRETARY, DEPARTMENT OF HEALTH & HUMAN SERVICES, Bethesda, MD (US)

(72) Inventors: Emily LEE, Rockville, MD (US); Caroline STRONG, Chevy Chase, MD (US); Molly BOUTIN, Troy, NY (US); Marc FERRER, Potomac, MD (US)

(21) Appl. No.: 18/547,037

(22) PCT Filed: Feb. 22, 2022
(86) PCT No.: PCT/US2022/017248

§ 371 Date: Aug. 18, 2023

Related U.S. Application Data
(60) Provisional application No. 63/151,698, filed on Feb. 20, 2021.

(51) Int. Cl. C12N 5/0793; C12N 5/079; G01N 33/50

(52) U.S. Cl. CPC C12N 5/0619; C12N 5/0622; G01N 33/5058; C12N 2502/081; C12N 2502/086; C12N 2502/28; C12N 2506/45; C12N 2513/00

(57) ABSTRACT

Functional, brain region-specific neural spheroids comprising neuronal cells and optionally glial cells at varying ratios are disclosed, as are methods of making such spheroids and methods for their use, such as for modeling particular brain regions that may be implicated in diseases, or for observing drug effects.

### BACKGROUND

### 1. Field

The disclosure relates to spheroids comprising neural cells and neural-associated cells that exhibit neurological properties including but not limited to electrophysiology, calcium activity, and neurotransmitter release. The disclosure further relates to methods of making and methods of using the spheroids.

### 2. Description of Related Art

Disease modeling and therapeutic testing of compounds for neurological diseases is particularly challenging because 2D in vitro culture models lack physiological relevance to in vivo neurocircuitry, while in vivo animal models are extremely low-throughput and have low human predictability.

Stem-cell derived neuronal organoids are used in modeling and therapeutic testing. However, several disadvantages severely limit the use of organoids for high-throughput systems (HTS). First, both cerebral and patterned brain organoids take long periods (weeks to months) to fully mature into different cell types and tissue-like architecture, and, second, their production protocols lack both robustness and reproducibility, producing organoids of variable size, cell composition and functionality, making them incompatible to use for HTS.

There exists a need in the art for in vitro preclinical models for neurological disorders and neurodegenerative diseases.

### BRIEF SUMMARY OF THE INVENTION

The present disclosure relates to the functional brain region-specific spheroids and their uses.

In an embodiment, an isolated spheroid may comprise a plurality of neurons. The spheroid may comprise between about 1% and 100% neurons by total number of cells in the spheroid, optionally about 90% neurons by total number of cells in the spheroid.

In an embodiment, the spheroid may further comprise at least one glial cell. The at least one glial cell may be an astrocyte, microglia, oligodendrocyte, and combinations thereof.

In an embodiment, the spheroid comprises between about 1% and 100% astrocytes by total number of cells in the spheroid.

In an embodiment, the spheroid may comprise between about 1% and 100% neurons by total number of neurons in the spheroid.

In an embodiment, the spheroid may further comprise endothelial cells.

In an embodiment, the spheroid may further comprise pericytes.

In an embodiment, the neurons may comprise afferent neurons, efferent neurons, interneurons, and combinations thereof.

In an embodiment, the neurons may comprise sensory neurons, motor neurons, interneurons, pyramidal neurons, and combinations thereof.

In an embodiment, the neurons may comprise unipolar neurons, bipolar neurons, pseudounipolar neurons, multipolar neurons, and combinations thereof.

In an embodiment, the neurons may comprise excitatory neurons, inhibitory neurons, and combinations thereof.

In an embodiment, the neurons may comprise GABAergic neurons, glutamatergic neurons, dopaminergic neurons, cholinergic neurons, serotonergic neurons, and combinations thereof.

In an embodiment, the spheroid may comprise GABAergic neurons, glutamatergic neurons, dopaminergic neurons, astrocytes, and combinations thereof. The spheroid may comprise GABAergic neurons, glutamatergic neurons, astrocytes, and combinations thereof. The spheroid may comprise GABAergic neurons, astrocytes, and combinations thereof. The spheroid may comprise motor neurons, GABAergic neurons, glutamatergic neurons, dopaminergic neurons, astrocytes, microglia, and combinations thereof. The spheroid may comprise motor neurons, GABAergic neurons, glutamatergic neurons, and combinations thereof. The spheroid may comprise motor neurons, GABAergic neurons, glutamatergic neurons, dopaminergic neurons, and combinations thereof. The spheroid may comprise motor neurons, GABAergic neurons, glutamatergic neurons, dopaminergic neurons, astrocytes, and combinations thereof. The spheroid may comprise motor neurons, GABAergic neurons, and combinations thereof. The spheroid may comprise motor neurons, glutamatergic neurons, and combinations thereof. The spheroid may comprise motor neurons, dopaminergic neurons, and combinations thereof. The spheroid may comprise motor neurons, astrocytes, and combinations thereof. The spheroid may comprise motor neurons, microglia, and combinations thereof. The spheroid may comprise GABAergic neurons, glutamatergic neurons, and combinations thereof. The spheroid may comprise GABAergic neurons, dopaminergic neurons, and combinations thereof. The spheroid may comprise GABAergic neurons, astrocytes, and combinations thereof. The spheroid may comprise GABAergic neurons, microglia, and combinations thereof. The spheroid may comprise glutamatergic neurons, dopaminergic neurons, and combinations thereof. The spheroid may comprise glutamatergic neurons, astrocytes, and combinations thereof. The spheroid may comprise glutamatergic neurons, microglia, and combinations thereof. The spheroid may comprise dopaminergic neurons, astrocytes, and combinations thereof. The spheroid may comprise dopaminergic neurons, microglia, and combinations thereof.

In an embodiment, between about 1 and 100% of the neurons in the spheroid may be motor neurons by total percentage of cell number per spheroid. The spheroid may comprise about 10% and 40% motor neurons by total number of cells, or between about 50% and 95% motor neurons by total number of cells, or between about 15% and 75% motor neurons by total number of cells. The spheroid may comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 91%, 92%, 93%, 94%, 95%, 96%, 97%, 98%, 99%, or 100% motor neurons by total number of cells.

In an embodiment, between about 1 and 100% of the neurons in the spheroid may be GABAergic neurons by total number of cells. The spheroid may comprise about 10% and 40% GABAergic neurons by total number of cells, or between about 50% and 95% GABAergic neurons by total number of cells, or between about 15% and 75% GABAergic neurons by total number of cells. The spheroid may comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 91%, 92%, 93%, 94%, 95%, 96%, 97%, 98%, 99%, or 100% GABAergic neurons by total number of cells.

In an embodiment, between about 1 and 100% of the neurons in the spheroid may be glutamatergic neurons by total percentage of cells per spheroid. The spheroid may comprise about 10% and 40% glutamatergic neurons by total number of cells, or between about 50% and 75% glutamatergic neurons by total number of cells, or between about 50% and 95% glutamatergic neurons by total number of cells. The spheroid may comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 91%, 92%, 93%, 94%, 95%, 96%, 97%, 98%, 99%, or 100% glutamatergic neurons by total number of cells.

In an embodiment, between about 1 and 100% of the cells in the spheroid may be dopaminergic neurons by total percentage of cell number per spheroid. The spheroid may comprise about 10% and 40% dopaminergic neurons by total number of cells, or between about 50% and 75% dopaminergic neurons by total number of cells, or between about 50% and 95% dopaminergic neurons by total number of cells. The spheroid may comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 91%, 92%, 93%, 94%, 95%, 96%, 97%, 98%, 99%, or 100% dopaminergic neurons by total number of cells.

In an embodiment, between about 1 and 100% of the cells in the spheroid may be astrocytes by total percentage of cell number per spheroid. The spheroid may comprise between about 1% and 20% astrocytes by total number of cells, or between about 5% and 25% astrocytes by total number of cells, or between about 10% and 75% astrocytes by total number of cells. The spheroid may comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 91%, 92%, 93%, 94%, 95%, 96%, 97%, 98%, 99%, or 100% astrocytes by total number of cells.

In an embodiment, between about 1 and 100% of the cells in the spheroid may be microglia by total percentage of cell number per spheroid. The spheroid may comprise between about 1% and 20% microglia by total number of cells, or between about 5% and 25% microglia by total number of cells, or between about 10% and 75% microglia by total number of cells. The spheroid may comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 91%, 92%, 93%, 94%, 95%, 96%, 97%, 98%, 99%, or 100% microglia by total number of cells.

In an embodiment, the percentage of neurons in the spheroid may be between about 1% and 20%, 10% and 20%, 15% and 60%, 20% and 40%, 50% and 80%, 40% and 90%, 25% and 50%, 35% and 65%, 5% and 70%, 65% and 70%, 60% and 98%, 1% and 50%, 5% and 75%, 10% and 40%, or 50% and 80% by total percentage of cell number per spheroid. The percentage of the neurons cells may be 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% by total percentage of cell number per spheroid.

In an embodiment, between about 1 and 100% of the neurons in the spheroid may be motor neurons by total percentage of neurons per spheroid. The spheroid may comprise between about 10% and 40% motor neurons by total number of neurons in the spheroid, or between about 50% and 95% motor neurons by total number of neurons in the spheroid, or between about 15% and 75% motor neurons by total number of neurons in the spheroid. The spheroid may comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 91%, 92%, 93%, 94%, 95%, 96%, 97%, 98%, 99%, or 100% motor neurons by total number of neurons in the spheroid.

In an embodiment, between about 1 and 100% of the neurons in the spheroid may be GABAergic neurons by total percentage of neurons per spheroid. The spheroid may comprise between about 10% and 40% GABAergic neurons by total number of neurons in the spheroid, or between about 50% and 95% GABAergic neurons by total number of neurons in the spheroid, or between about 15% and 75% GABAergic neurons by total number of neurons in the spheroid. The spheroid may comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 91%, 92%, 93%, 94%, 95%, 96%, 97%, 98%, 99%, or 100% GABAergic neurons by total number of neurons in the spheroid. About 30% of the neurons in the spheroid may be GABAergic neurons.

In an embodiment, between about 1 and 100% of the neurons in the spheroid may be glutamatergic neurons by total percentage of neurons per spheroid. The spheroid may comprise about 10% and 40% glutamatergic neurons by total number of neurons in the spheroid, or between about 50% and 75% glutamatergic neurons by total number of neurons in the spheroid, or between about 50% and 95% glutamatergic neurons by total number of neurons in the spheroid. The spheroid may comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 91%, 92%, 93%, 94%, 95%, 96%, 97%, 98%, 99%, or 100% glutamatergic neurons by total number of neurons in the spheroid. About 5% to 70% of the neurons in the spheroid may be glutamatergic neurons by total percentage of neurons per spheroid.

In an embodiment, between about 1 and 100% of the neurons in the spheroid may be dopaminergic neurons by total percentage of neurons per spheroid. The spheroid may comprise about 10% and 40% dopaminergic neurons by total number of neurons in the spheroid, or between about 50% and 75% dopaminergic neurons by total number of neurons in the spheroid, or between about 50% and 95% dopaminergic neurons by total number of neurons in the spheroid. The may comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 91%, 92%, 93%, 94%, 95%, 96%, 97%, 98%, 99%, or 100% dopaminergic neurons by total number of neurons in the spheroid. About 65% of the neurons in the spheroid may be dopaminergic neurons.

In an embodiment, the spheroid may further comprise endothelial cells. The amount of endothelial cells may be between 1% and 100% by total number of cells. The spheroid may comprise between about 10% and 40% endothelial cells by total number of cells, or between about 50% and 75% endothelial cells by total number of cells, or between about 50% and 95% endothelial cells by total number of cells. The spheroid may comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 91%, 92%, 93%, 94%, 95%, 96%, 97%, 98%, 99%, or 100% endothelial cells by total number of cells.

In an embodiment, the spheroid may further comprise pericytes. The amount of pericytes may be between 1% and 100% by total number of cells. The spheroid may comprise between about 10% and 40% pericytes by total number of cells, or between about 50% and 75% pericytes by total number of cells, or between about 50% and 95% pericytes by total number of cells. The spheroid may comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 91%, 92%, 93%, 94%, 95%, 96%, 97%, 98%, 99%, or 100% pericytes by total number of cells.

In an embodiment, between about 1% and 100% of the cells may be neurons by total number of cells. The spheroid may comprise about 10% and 40% neurons by total number of cells, or between about 50% and 75% neurons by total number of cells, or between about 50% and 95% neurons by total number of cells. The spheroid may comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 91%, 92%, 93%, 94%, 95%, 96%, 97%, 98%, 99%, or 100% neurons by total number of cells.

In an embodiment, the spheroid may comprise GABAergic neurons, glutamatergic neurons, astrocytes in a ratio of 7 to 7.5 glutamatergic neurons: 2.5 to 3 GABAergic neurons: 1 astrocyte. The spheroid may comprise GABAergic neurons, glutamatergic neurons, dopaminergic neurons, and astrocytes in a ratio of 3 to 3.5 GABAergic neurons: 0.5 glutamatergic neurons: 6.0 to 6.5 dopaminergic neurons: and 1 astrocyte.

In an embodiment, the spheroid may be a VTA-like spheroid comprising 65% Dopaminergic neurons, 5% glutamatergic neurons, 30% GABAergic neurons by percentage of neurons and 10% astrocytes by total number of cells.

In an embodiment, the spheroid may be a PFC-like spheroid comprising 0% dopaminergic neurons, 70% glutamatergic neurons, 30% GABAergic neurons by percentage of neurons and 10% astrocytes by total number of cells.

In an embodiment, the spheroid may comprise about 65% dopaminergic neurons, about 30% GABAergic neurons, and about 5% glutamatergic neurons by total percentage of cell number per spheroid. The spheroid may comprise about 30% GABAergic neurons and about 70% glutamatergic neurons by total percentage of cell number per spheroid. The spheroid may comprise about 90% dopaminergic neurons and about 10% astrocytes by total percentage of cell number per spheroid. The spheroid may comprise about 90% GABAergic neurons and about 10% astrocytes by total percentage of cell number per spheroid. The spheroid of any one of claims 1-75, wherein the spheroid may comprise about 90% glutamatergic neurons and about 10% astrocytes by total percentage of cell number per spheroid. The spheroid may comprise about 85% dopaminergic neurons about 5% GABAergic neurons, and about 10% astrocytes by total percentage of cell number per spheroid. The spheroid may comprise about 75% dopaminergic neurons, about 15% GABAergic neurons, and about 10% astrocytes by total percentage of cell number per spheroid. The spheroid may comprise about 85% GABAergic neurons, about 5% dopaminergic neurons, and about 10% astrocytes by total percentage of cell number per spheroid. The spheroid may comprise about 75% GABAergic neurons, about 15% dopaminergic neurons, and about 10% astrocytes by total percentage of cell number per spheroid. The spheroid may comprise about 30% dopaminergic neurons, about 30% GABAergic neurons, about 30% glutamatergic neurons, and about 10% astrocytes by total percentage of cell number per spheroid. The spheroid may comprise about 45% GABAergic neurons, about 45% glutamatergic neurons, and about 10% astrocytes by total percentage of cell number per spheroid. The spheroid may comprise about 45% dopaminergic neurons, about 45% glutamatergic neurons, and about 10% astrocytes by total percentage of cell number per spheroid. The spheroid may comprise about 75% dopaminergic neurons, about 7.5% GABAergic neurons, about 7.5% glutamatergic neurons, and about 10% astrocytes by total percentage of cell number per spheroid. The spheroid may comprise about 55% dopaminergic neurons, about 15% GABAergic neurons, about 15% glutamatergic neurons, and about 10% astrocytes by total percentage of cell number per spheroid. The spheroid may comprise about 22.5% dopaminergic neurons, about 45% GABAergic neurons, about 22.5% glutamatergic neurons, and about 10% astrocytes by total percentage of cell number per spheroid. The spheroid may comprise about 7.5% dopaminergic neurons, about 75% GABAergic neurons, about 7.5% glutamatergic neurons, and about 10% astrocytes by total percentage of cell number per spheroid. The spheroid may comprise about 22.5% dopaminergic neurons, about 22.5% GABAergic neurons, about 45% glutamatergic neurons, and about 10% astrocytes by total percentage of cell number per spheroid. The spheroid may comprise about 7.5% dopaminergic neurons, about 7.5% GABAergic neurons, about 75% glutamatergic neurons, and about 10% astrocytes by total percentage of cell number per spheroid.

In an embodiment, the spheroid may comprise about 60% dopaminergic neurons, about 27.5% GABAergic neurons, about 2.5% glutamatergic neurons, and about 10% astrocytes by total percentage of cell number per spheroid and exhibits the properties of cells from the ventral tegmental area (VTA).

In an embodiment, the spheroid may comprise about 25% GABAergic neurons, about 65% glutamatergic neurons, and about 10% astrocytes by total percentage of cell number per spheroid and exhibits the properties of cells from the prefrontal cortex (PFC).

In an embodiment, the spheroid may comprise between about 1 and 100,000 cells in total. The spheroid may comprise between about 100 and 100,000 cells in total; 5,000 and 30,000 cells in total; 1,000 and 50,000 cells in total; 10,000 and 25,000 cells in total; 25,000 and 50,000 cells in total; 5,000 and 10,000 cells in total; 30,000 and 70,000 cells in total; or 15,000 and 30,000 cells in total. The spheroid may comprise about 1,000; 2,000; 3,000; 4,000; 5,000; 6,000; 7,000; 8,000; 9,000; 10,000; 11,000; 12,000; 13,000; 14,000; 15,000; 16,000; 17,000; 18,000; 19,000; 20,000; 21,000; 22,000; 23,000; 24,000; 25,000; 26,000; 27,000; 28,000; 29,000; 30,000; 31,000; 32,000; 33,000; 34,000; 35,000; 36,000; 37,000; 38,000; 39,000; 40,000; 41,000; 42,000; 43,000; 44,000; 45,000; 46,000; 47,000; 48,000; 49,000; 50,000; 51,000; 52,000; 53,000; 54,000; 55,000; 56,000; 57,000; 58,000; 59,000; 60,000; 61,000; 62,000; 63,000; 64,000; 65,000; 66,000; 67,000; 68,000; 69,000; 70,000; 71,000; 72,000; 73,000; 74,000; 75,000; 76,000; 77,000; 78,000; 79,000; 80,000; 81,000; 82,000; 83,000; 84,000; 85,000; 86,000; 87,000; 88,000; 89,000; 90,000; 91,000; 92,000; 93,000; 94,000; 95,000; 96,000; 97,000; 98,000; 99,000; and 100,000 cells in total. The spheroid may comprise about 10,000 cells in total. The spheroid may comprise about 30,000 cells in total.

In an embodiment, the spheroid may exhibit electrophysiological properties, calcium activity profile, neurotransmitter release, or a combination thereof, substantially similar to cells from a defined brain region selected from the ventral tegmental area (VTA), prefrontal cortex (PFC), nucleus accumbens, amygdala, hippocampus, somatomotor cortex, somatosensory cortex, parietal lobe, occipital lobe, cerebellum, and temporal lobe.

In an embodiment, the spheroid may exhibit electrophysiological properties. The spheroid may exhibit electrophysiological properties substantially similar to cells in the ventral tegmental area (VTA). The spheroid may exhibit electrophysiological properties substantially similar to cells in the prefrontal cortex (PFC). The spheroid may exhibit electrophysiological properties substantially similar to cells in the nucleus accumbens. The spheroid may exhibit electrophysiological properties substantially similar to cells in the amygdala. The spheroid may exhibit electrophysiological properties substantially similar to cells in the hippocampus.

In an embodiment, the spheroid may exhibit a calcium activity profile. The spheroid may exhibit calcium activity profiles substantially similar to cells in the ventral tegmental area (VTA). The spheroid may exhibit calcium activity profiles substantially similar to cells in the prefrontal cortex (PFC). The spheroid may exhibit calcium activity profiles substantially similar to cells in the nucleus accumbens. The spheroid may exhibit calcium activity profiles substantially similar to cells in the amygdala. The spheroid may exhibit calcium activity profiles substantially similar to cells in the hippocampus.

In an embodiment, the spheroid may exhibit neurotransmitter release. The spheroid may exhibit neurotransmitter release substantially similar to cells in the ventral tegmental area (VTA). The spheroid may exhibit neurotransmitter release substantially similar to cells in the prefrontal cortex (PFC). The spheroid may exhibit neurotransmitter release substantially similar to cells in the nucleus accumbens. The spheroid may exhibit neurotransmitter release substantially similar to cells in the amygdala. The spheroid may exhibit neurotransmitter release substantially similar to cells in the hippocampus.

In an embodiment, the spheroid may be between about 10 m and 1,000 m in size, as measured across the diameter. The spheroid may be between about 100 m and 500 m in size, as measured across the diameter, between about 250 m and 725 m in size, as measured across the diameter, or between about 750 m and 1,000 m in size as measured across the diameter. The spheroid may be about 100, 125, 150, 175, 200, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or 1,000 m in size as measured across the diameter.

In an embodiment, the spheroid may be substantially spherical in shape.

In an embodiment, the spheroid may grow in suspension.

In an embodiment, the spheroid may not adhere to a substrate in culture.

In an embodiment, the neurons may be differentiated.

In an embodiment, the glia may be differentiated.

In an embodiment, a method of making a spheroid described herein may comprise: (a) obtaining neurons; (b) admixing the neurons; and (c) culturing the admixed neurons under conditions to form a spheroid. The method may further comprise adding glial cells. The neurons obtained in step (a) may be differentiated neurons. The method may comprise admixing the neurons and/or glia in step (b) at a pre-determined amount. The method may comprise agitating the neurons and/or glia admixed for between about 1 and 10 minutes. The neurons and/or glia may be cultured in step (c) at about 37° C. The neurons and/or glia may be centrifuged after step (b) and before step (c). The spheroid may be cultured in step (c) for between about 7-28 days after admixing the cells together, optionally for about 21 days. The spheroid may mature in about 7-28 days after spheroid formation, optionally after about 21 days. The media used in step (c) may comprise N2 media supplement, B27 media supplement, brain-derived neurotrophic factor (BDNF), glial cell line-derived neurotrophic factor (GDNF), laminin, ascorbic acid, cAMP, and combinations thereof.

In an embodiment, a method of making a spheroid described herein may comprise: (a) obtaining cells comprising neurons, glia, and combinations thereof, optionally wherein the neural cells may be differentiated neural cells; (b) admixing the cells; (c) providing agitation to the mixture of neural cells; (d) centrifuging the cells; (e) resuspending the cells after centrifugation; (f) plating the cells in a vessel; and (g) culturing the cells under conditions to form a spheroid. The vessel may be a plate, dish, tray, or flask. The well may be a multi-well dish.

In an embodiment, a method of using the spheroid of any one of the above embodiments, comprising culturing the spheroid and measuring electrophysiological activity in the presence and absence of an agent. The agent may comprise at least one compound or a combination of two or more compounds. The agent may comprise at least one control compound and at least one test compound. The compound may be a toxin. The agent may be a dopamine receptor agonist, dopamine receptor antagonist, glutamate receptor agonist, glutamate receptor antagonist, GABA receptor agonist, GABA receptor antagonist, opioid receptor agonist, opioid receptor antagonist, and combinations thereof.

### BRIEF DESCRIPTION OF THE DRAWINGS

FIG. 1 depicts an overview of how region-specific spheroids described herein may be assembled. Stem cells, here induced human pluripotent stem cells (hiPSCs), may be differentiated into neurons, e.g., motor neurons, GABAergic neurons, glutamatergic neurons, dopaminergic neurons, and/or glial cells, e.g., astrocytes, microglia. The region-specific spheroids described herein are assembled by combining differentiated neural cells in different amounts. The methods described herein allow for the study of genetic influences by combining healthy and diseased cells.

FIG. 2, panel A depicts the formation of uniform and functional spheroids from fully matured, iPSC-derived neurons and iPSC derived astrocytes, 3 weeks post-formation. Spheroids of different compositions of iPSC-derived dopaminergic neurons, GABAergic neurons, glutamatergic neurons, and astrocytes all form viable spheroids using the disclosed formation protocol. Each column is a different ratio of iPSC derived neurons (dopaminergic:GABAergic:glutamatergic:10% astrocytes). 384-well plate. FIG. 2, panel B depicts a single spheroid as described herein, comprising about 10,000 neural cells, substantially spherical shape, and approximate diameter of about 450-500 μm. The scale bar is 1,000 μm.

FIG. 3 depicts a mapping of exemplary region-specific spheroids as described herein and their corresponding area in the mammalian brain (a human brain is shown for illustrative purposes). The spheroids described herein can be designed and produced to model relevant brain regions by combining differentiated neurons, including iPSC-derived differentiated neurons, and used to study scientifically- and clinically-relevant topics including (but not limited to) opioid use disorder (OUD), Parkinson's disease (PD), Alzheimer's disease (AD), Huntington's Disease (HD), Autism spectrum disorder, Rett Syndrome, Dravet Syndrome, dementia, epilepsy, Amyotrophic lateral sclerosis, or Down Syndrome.

FIG. 4 depicts 16 exemplary spheroids as described herein and their activity profiles. The spheroids shown in this figure were formed, maintained in culture for about 4 weeks, and then calcium activity was tested using a fluorescent imaging plate reader (FLIPR). Calcium 6 (Cal6), a dye that fluoresces as intracellular calcium fluctuates, was added to spheroids 2-hours (hrs) before recording on the FLIPR. Spheroid activity was then measured by examining changes in intracellular calcium levels, which is used as a readout for neuronal activity. Here it was shown that population neuronal activity within spheroids is unique to each ratio of designer spheroid since activity profiles were different between the 16 spheroids, each with a different neurons and/or glia composition, was tested. GABAergic neurons, glutamatergic neurons, dopaminergic neurons, astrocytes. (1)-(16) depict a spheroid with different mixture of neurons and glia.

FIG. 5 depicts the spheroid calcium activity, as measured by FLIPR, between StemoniX cortical spheroids (data obtained from Woodruff, G.; Phillips, N.; Carromeu, C.; et al. Screening for Modulators of Neural Network Activity in 3D Human iPSC-derived Cortical Spheroids. PLoS One. 2020, 15(10), e0240991.) and prefrontal cortex (PFC)-like spheroids described herein. (A) Baseline FLIPR recordings form StemoniX cortical spheroids in Supplementary FIG. 1 of of Woodruff et al. PLoS One (2020), 15, e0240991. Here, neurons were transduced with Incucyte Neuroburst Orange reagent, a lentivirus encoding a fluorescent calcium indicator driven by the synapsin promoter was used in order to examine calcium activity changes within neurons. 10-minute baseline recordings were obtained at 8-weeks and 12-weeks, and representative calcium activity plots over the 10-min recording are displayed. (B). A 10-min baseline FLIPR recording in 3-week old PFC-like spheroids, which were exposed to cal6 dye, which fluctuates in fluorescence as intracellular calcium activity levels change. Representative plot of 3-week old PFC-like spheroid is shown, with calcium activity over the 10-min recording displayed. This activity was measured in prefrontal-cortex-like spheroids, which were 90% neurons and 10% astrocytes, with neurons comprising 70% glutamatergic neurons and 30% GABAergic neurons as a percentage of total neurons after 3 weeks in culture.

FIG. 6 depicts a flow chart with an exemplary method for making the spheroids described herein.

FIG. 7 depicts a flow chart with an exemplary method for characterization and use of the spheroids described herein. The last three steps may be modified to fit a specific application and are extendable to other disorders/diseases, e.g., neurodegenerative disorders including but not limited to Alzheimer's Disease, Parkinson's disease, and Huntington's Disease.

FIG. 8 depicts exemplary data acquisition and filtering pipelines. Step 1. Prior to beginning FLIPR recording, an image of the cell plate is obtained to view which spheroids are inside or outside of the mask where fluorescence is imaged by the FLIPR camera. Step 2. Representative plots showing calcium activity from spheroids inside the mask vs outside of the mask. Step 3. After FLIPR recordings, analysis is done within PeakPro 2.0 software to extract 17 parameters associated with spheroid calcium activity peaks. Percent coefficients of variance (CV) values are calculated to determine which peak parameters to use in the high content profiler (HCP) analysis. Examples of some, but not all, of these parameters are shown in the graphic below. Step 4. Peak parameters obtained from PeakPro 2.0 that contained CV values below 20% were incorporated into the HCP analysis. Principal component analysis generated a 3D scatter plot showing the spatial distribution of designer spheroids based on calcium activity profiles. Here it is shown that spheroids consisting only of GABAergic, glutamatergic, and dopaminergic neurons are separated away from each other. Also, PFC-like spheroids (70% glutamatergic, 30% GABAergic neurons) cluster in between spheroids with Gluta Only or GABA Only while VTA-like spheroids (65% dopaminergic, 5% glutamatergic, 30% GABAergic neurons) cluster closer to spheroids with Dopa Only. As a control, another designer spheroid type containing 60% dopaminergic, 20% glutamatergic, and 20% GABAergic neurons was also examined, and calcium activity profiles from the HCP analysis clustered it closest to VTA-like and Dopa Only spheroids. This shows that calcium activity profiles from designer spheroids containing similar ratios and cell types cluster closely together.

FIG. 9, panel A depicts the activity of ventral tegmental area (VTA)-like spheroids comprising 65% dopaminergic neurons, 30% GABAergic neurons, and 5% glutamatergic neurons as a percentage of total neurons and 10% astrocytes by total number of cells. Data shown here is from a 10-min FLIPR recording 1-min after spheroids were tested with DMSO and quality control (QC) compounds, including voltage-gated potassium (Kv) inhibitor, GABA_{A} receptor antagonist and agonist, dopamine receptor D2 antagonist, dopamine receptor D1 antagonist, NMDA receptor antagonist, and AMPA receptor antagonist. Peak parameters used in the high content profiler analysis were examined here individually, and QC compounds were compared to DMSO-control wells. In VTA-like spheroids, Kv-inhibition increases peak decay time. GABA_{A} receptor antagonism has stimulatory effects, increasing peak count, amplitude, rate, and reducing spacing and rise time. GABA_{A} receptor agonism, along with D1R and AMPAR antagonism reliably produce inhibitory effects in spheroids. Antagonism of D2Rs, which are inhibitory g-protein coupled receptors, showed a statistical trend toward a significant increase in peak count. These data show that GABA_{A} receptor antagonism in this spheroid type serves as a good stimulatory control compound while GABA_{A} receptor agonism serves as a good negative control compound.

FIG. 9, panel B depicts the activity of prefrontal cortex (PFC)-like spheroids comprising 0% dopaminergic neurons, 30% GABAergic neurons, and 70% glutamatergic neurons as a percentage of total neurons and 10% astrocytes by total number of cells. Data shown here is from a 10-min FLIPR recording 1-min after spheroids were tested with DMSO, and quality control (QC) compounds, including Kv inhibitor, GABA_{A} receptor antagonist and agonist, dopamine receptor D2 antagonist, dopamine receptor D1 antagonist, NMDA receptor antagonist, and AMPA receptor antagonist. Peak parameters used in the high content profiler analysis were examined here individually, and QC compounds were compared to DMSO-control wells. In PFC-like spheroids, Kv-inhibition reliably increases peak count. GABAaR antagonism increased peak amplitude, rate, rise and decay time, while decreasing peak rate, suggesting a complex activity profile indicative of both excitatory and inhibitory effects. GABAaR agonism, along with D1R and AMPAR antagonism reliably produce inhibitory effects in spheroids. These data show that Kv inhibition in this spheroid type serves as a good stimulatory control compound while GABAaR agonism serves as a good negative control compound.

FIG. 10: panel A depicts the baseline activity of ventral tegmental area (VTA)-like spheroids comprising 65% dopaminergic neurons, 30% GABAergic neurons, and 5% glutamatergic neurons as a percentage of total neurons and prefrontal cortex (PFC)-like spheroids (spheroids described herein comprising 0% dopaminergic neurons, 30% GABAergic neurons, and 70% glutamatergic neurons as a percentage of total neurons and 10% astrocytes by total number of cells) with chronic DAMGO ([D-Ala², N-MePhe⁴, Gly-ol]-enkephalin) treatment. Top Row (A)=control wells. Middle Row (A)=chronically dosed with 10 μM DAMGO for 10 days. Bottom Row (A)=chronically dosed with 10 μM DAMGO for 7 days, followed by 3 days withdrawal. This models opioid use disorder where chronic DAMGO treatment has the opposite effect on peak count (panel B) in VTA-like versus PFC-like spheroids while reducing peak amplitude (panel C) in both spheroid groups. Chronic DAMGO treatment (with and without withdrawal (“WD”)) has a differential effect on peak decay time in VTA-like spheroids versus PFC-like spheroids. The baseline changes after chronic DAMGO treatment and DAMGO withdrawal in VTA-like spheroids are consistent with data from in vivo animal and human studies, showing increased basal activity within this brain region. Human studies show elevated BOLD signal activation within the VTA of people suffering from heroin addiction in response to visual cues related to heroin, when compared to the VTA of healthy individuals (Yang et al. Human Brain Mapp. 2009, 30(3):766-75; Zijlstra et al. Drug Alcohol Depend. 2009, 99(1-3): 183-92). In animal studies, chronic morphine exposure in mice increases the basal firing rate of dopaminergic neurons in the VTA, measured by patch clamp electrophysiology (Meye et al. J Neurosci. 2012, 32(46):16120-8). In vivo electrophysiology data suggests heroin self-administration inhibits prefrontal cortical neurons (Chang et al. Brain Res. (1997) 754(1-2):12-20; Nui et al. Neurosci Lett. 2017, 640:144-151). Furthermore, chronic morphine is associated with dendritic spine loss within glutamatergic pyramidal neurons within the PFC, suggesting reduced glutamatergic transmission and enhanced inhibition within this brain region (Robinson et al. Synapse (1999) 33(2): 160-2).

FIG. 11 (panels A-C) depicts calcium activity profiles according to the type of spheroid, obtained from 8-minute FLIPR recordings. These data show unique calcium activity profiles dependent on neuronal subtypes present in the spheroid. (A) Dopaminergic neurons+astrocytes; (B) GABAergic neurons+astrocytes; and (C) glutamatergic neurons+astrocytes. All were measured at about 3-weeks of culture.

FIG. 12 depicts functional profiles visualized using calcium imaging with FLIPR® (fluorometric imaging plate reader) for 3-week-old spheroids formed using iPSC-derived neurons and astrocytes as described herein. Each column represents an individual spheroid with a specific cell composition.

FIG. 13 (panels A-C) depicts distinct activity profiles between VTA-like and PFC-like spheroids. (A) VTA-like spheroids are compared to PFC-like spheroids to show distinct calcium activity profiles between these two populations. PFC-like spheroids, consisting of majority excitatory glutamatergic neurons (70% of total neuron population) show increased excitability compared to VTA-like neurons, which are primarily dopaminergic neurons (65%). (B) Peak parameters obtained from PeakPro 2.0 that contained CV values below 20% were incorporated into the HCP analysis. Principal component analysis generated a 3D scatter plot showing the spatial distribution of designer spheroids based on calcium activity profiles. Here it is shown that spheroids consisting only of GABAergic, glutamatergic, and dopaminergic neurons are separated away from each other. Also, PFC-like spheroids (70% glutamatergic, 30% GABAergic neurons) cluster in between spheroids with Gluta Only or GABA Only while VTA-like spheroids (65% dopaminergic, 5% glutamatergic, 30% GABAergic neurons) cluster closer to spheroids with Dopaminergic (Dopa) Only. As a control, another designer spheroid type containing 60% dopaminergic, 20% glutamatergic, and 20% GABAergic neurons was also examined, and calcium activity profiles from the HCP analysis clustered it closest to VTA-like and Dopa Only spheroids. This shows that calcium activity profiles from designer spheroids containing similar ratios and cell types cluster closes together. (C) Depicts light microscopy imaging of a spheroid, showing spheroid shape and size of a spheroid consisting of 10,000 cells. The scale bar is 1,000 μm (1 mm).

FIG. 14 (panels A-D) depicts activity profiles in response to selected compound classes (DMSO, Kv inhibitor, GABA_{A} receptor antagonist and agonist, dopamine receptor D2 antagonist, dopamine receptor D1 antagonist, NMDA receptor antagonist, and AMPA receptor antagonist) in ventral tegmental area (VTA)-like spheroids (spheroids described herein comprising 65% dopaminergic, 30% GABAergic, and 5% glutamatergic neurons as a percentage of total neurons and 10% astrocytes by total number of cells). Baseline=spheroid calcium activity prior to compound exposure; t1=spheroid calcium activity 1-min after treatment with compounds; t30=spheroid calcium activity 30-min after treatment with compounds; t70=spheroid calcium activity 70-min after treatment with compounds.

FIG. 15 (panels A-D) depicts activity profiles in response to selected compound classes (DMSO, Kv inhibitor, GABA_{A} receptor antagonist and agonist, dopamine receptor D2 antagonist, dopamine receptor D1 antagonist, NMDA receptor antagonist, and AMPA receptor antagonist) in prefrontal cortex (PFC)-like spheroids (spheroids described herein comprising 0% dopaminergic, 30% GABAergic, and 70% glutamatergic neurons as a percentage of total neurons and 10% astrocytes by total number of cells). Baseline=spheroid calcium activity prior to compound exposure; t1=spheroid calcium activity 1-min after treatment with compounds; t30=spheroid calcium activity 30-min after treatment with compounds; t70=spheroid calcium activity 70-min after treatment with compounds.

FIG. 16 (panels A-D) shows representative plots for VTA-like spheroids at baseline, along with 1- and 30-min after exposure to either DMSO (left) or DAMGO (right), and 30-min (70-min after initial compound treatment) after exposure to either DMSO (left) or naloxone (right). B-D depict peak count, peak amplitude, and peak decay time with for VTA-like spheroids (spheroids described herein comprising 65% dopaminergic, 30% GABAergic, and 5% glutamatergic neurons as a percentage of total neurons) acutely treated with DAMGO for the first time (Control), acutely treated with DAMGO after previously being chronically treated with DMAGO (Chronic), and acutely treated with DAMGO after being chronically treated and subjected to a 3-day withdrawal period (Chronic+withdrawal). (16A) depicts control, chronic DAMGO, and DAMGO withdrawal effects on waveform. Black [1]=control wells. Green [2]=chronically dosed with 10 μM DAMGO for 10 days. Blue [3]=chronically dosed with 10 μM DAMGO for 7 days, followed by 3 days of withdrawal. (FIG. 16B) In control spheroids, neither DAMGO nor naloxone impacted peak count; In Chronic spheroids, DAMGO increased peak count 1- and 30-min after exposure, and naloxone reduced this to control levels; In Chronic+withdrawal spheroids, DAMGO did not affect peak count but naloxone reduced it to control levels. (16C) In control spheroids, DAMGO increased peak amplitude 1-min after treatment and naloxone increased it 30-min after treatment; In Chronic spheroids, DAMGO did not impact peak amplitude but naloxone increased it to control levels; Neither DAMGO nor naloxone had an impact on peak amplitude in chronic+withdrawal spheroids. (16D) In control spheroids, naloxone increases peak decay time; In chronic DAMGO-treated spheroids and chronic-DAMGO treated spheroids subjected to 3-days withdrawal, acute DAMGO exposure decreases peak decay time and naloxone rescues this back to control levels; depicts control, chronic, and chronic+withdrawal for peak decay time for VTA-like spheroids. For 16B-16D, baseline=activity prior to DAMGO/naloxone treatment; 1 min=1 minute after DAMGO; 30 min=30 minutes after DAMGO; 70 min=30 minutes after naloxone. Acute DAMGO increases peak count and decreases peak decay time in chronic DAMGO-treated wells. Blocking mu opioid receptors with naloxone rescues peak count and peak decay time.

FIG. 17 (panels A-D) depicts peak count, peak amplitude, and peak decay time with DMSO control and DAMGO treatment for PFC-like spheroids (spheroids comprising 0% dopaminergic neurons, 30% GABAergic neurons, and 70% glutamatergic neurons as a percentage of total neurons) acutely treated with DAMGO for the first time (Control), acutely treated with DAMGO after previously being chronically treated with DMAGO (Chronic), and acutely treated with DAMGO after being chronically treated and subjected to a 3-day withdrawal period (Chronic+withdrawal). (17A) depicts control, chronic DAMGO, and DAMGO withdrawal effects on waveform. Black [1]=control wells. Green [2]=chronically dosed with 10 μM DAMGO for 10 days. Blue [3]=chronically dosed with 10 μM DAMGO for 7 days, followed by 3 days of withdrawal. (17B) In control spheroids, DAMGO decreases peak count while naloxone rescues this; Neither DAMGO nor naloxone impact peak count in chronic spheroids; In chronic+withdrawal spheroids, DAMGO reduces, but naloxone does not impact, peak count; depicts control, chronic, and chronic+withdrawal for peak count for PFC-like spheroids. (17C) In control spheroids, DAMGO increases peak amplitude and naloxone increases this further; in chronic spheroids, DAMGO reduces peak amplitude and naloxone rescues this to control levels; in chronic+withdrawal spheroids, naloxone rescues deficits induced by DAMGO withdrawal; depicts control, chronic, and chronic+withdrawal for peak amplitude for PFC-like spheroids. (17D) depicts control, chronic, and chronic+withdrawal for peak decay time for PFC-like spheroids. For 17B-17D, baseline=activity prior to DAMGO/naloxone treatment; 1 min=1 minute after DAMGO; 30 min=30 minutes after DAMGO; 70 min=30 minutes after naloxone. Acute DAMGO decreases peak amplitude in chronic DAMGO-treated wells. Blocking mu opioid receptors with naloxone rescues peak amplitude.

FIG. 18 shows that chronic DAMGO treatment and DAMGO withdrawal differentially shifts peak count frequency distributions in VTA-like and PFC-like spheroids. The following timeline was used: Day 1: Formation of spheroids in 384-well ULA plates; Day 11: Begin dosing with 10 μM DAMGO, media replenish every other day; Day 17: Remove DAMGO from withdrawal wells; and Day 21: Record activity.

FIGS. 19A and 19B depict calcium profiles measured in spheroids. FIG. 19A shows VTA-like spheroids calcium profiles after immediate (T1) or 70 minutes post treatment (T70) with the above-mentioned QC compounds of known MOA. FIG. 19B shows PFC-like spheroids calcium profiles after immediate (T1) or 70 minutes post treatment (T70) with the above-mentioned QC compounds of known MOA.

FIG. 20 depicts a designer spheroid (in this case, VTA-like spheroid) that has successfully been transduced with a retrograde adeno-associated virus (AAVRG) that overexpresses mCherry fluorescent protein driven by a human synapsin promoter.

FIG. 21A depicts Raw Fluorescence Units (RFU) (%) [y-axis] and Time (seconds) [x-axis] for a variety of spheroids described herein. Representative traces from 16 different spheroids that are each 90% neuron and 10% astrocyte but differ by neuronal subtype composition; neuronal subtype composition can be seen in the figure. The spheroids included 100% dopaminergic neurons; 90% dopaminergic (dopa): 10% GABAergic (GABA); 80% dopaminergic (dopa): 20% GABAergic (GABA); 80% dopaminergic (dopa): 10% glutaminergic (gluta): 10% GABAergic (GABA); 60% dopaminergic (dopa): 20% glutaminergic (gluta): 20% GABAergic (GABA); 50% dopaminergic (dopa): 50% Glutaminergic (gluta); 33% dopaminergic (dopa): 33% glutaminergic (gluta): 33% GABAergic (GABA); 25% dopaminergic (dopa): 25% glutaminergic (gluta): 50% GABAergic (GABA); 100% glutaminergic (gluta); 10% dopaminergic (dopa): 80% glutaminergic (gluta): 10% GABAergic (GABA); 25% dopaminergic (dopa): 50% glutaminergic (gluta): 25% GABAergic (GABA); 50% glutaminergic (gluta): 50% GABAergic (GABA); 10% dopaminergic (dopa): 10% glutaminergic (gluta): 80% GABAergic (GABA); 20% dopaminergic (dopa): 80% GABAergic (GABA); 20% dopaminergic (dopa): 80% GABAergic (GABA); 10% dopaminergic (dopa): 90% GABAergic (GABA); and 100% GABAergic (GABA), the percentages are out of total number of neurons in the spheroid (90% neurons and 10% astrocytes as total number of cells in the spheroid). Spheroids were maintained for 3-weeks after generation and activity was recorded with the FLIPR.

FIG. 21B depicts Principal component analysis (PCA) that was used as a dimension reduction algorithm to incorporate 10 peak parameters extracted from the multiparametric peak analysis for all wells, and scatter plots were used to visualize the spatial distribution of calcium activity phenotypes for each spheroid type. Plots from top to bottom: 1. single neuron spheroid (SNS) types (100% dopaminergic, glutamatergic, or GABAergic neurons) 2. Spheroids with majority dopaminergic neurons (shades of blue) relative to SNSs 3. spheroids with mostly GABAergic neurons (shades of orange) relative to SNSs 4. spheroids with mostly glutamatergic neurons (shades of pink) relative to SNSs 5. spheroids with equal distributions of neuronal cell types (Data was obtained from 16 technical replicates per spheroid type over one experiment; Raw data obtained from the multiparametric peak analysis was used for the PCA; For (B), individual data points for each spheroid type are shown).

FIGS. 22A, 22B and 22C depict that synchronous calcium oscillations occur in brain region-specific spheroids as well as single neuron spheroids with dopaminergic and glutamatergic, but not GABAergic, neurons. Experimental design: spheroids were incubated in Cal6 dye for 2-hrs prior to confocal time series recordings after a 3-week maintenance period. (22A) Calcium activity in brain region-specific spheroids modeling the ventral tegmental area (VTA-like) and prefrontal cortex (PFC-like); Top: visual of the automated region of interest (ROI) detection used to examine calcium activity within several cells in each spheroid; Second from top: calcium activity from all identified ROIs shown in the form of a heatmap, with tick marks for ROIs in increments of 5 on the left y-axis, dF/F0 on the right y-axis, and video frame on the x-axis; Second from bottom: Average signal of all identified ROIs plus 95% confidence interval; Bottom: Correlation matrices displaying R2 values as a heatmap for all identified ROIs within a spheroid; (22B) Correlation matrices from representative single neuron spheroids showing synchrony of all identified ROIs (22C) Quantification of synchrony through a correlation score, obtained through the mean R2 value for each spheroids' correlation matrix. (Data was run in technical replicates of n=4-6 per plate across one experiment for SNSs and three separate experiments for brain region specific spheroids; For (22C), One way ANOVA (F_{(4,35)}=100.7, p<0.0001) was followed up with Tukey's posthoc (p<0.0001 for all groups compared to GABA+Astro spheroids), data is represented as mean±SEM, *** p<0.001.

FIGS. 22D and 22E show that astrocytes are not necessary for neuronal activity, but their presence alters phenotypic profiles of single neuron spheroids (SNSs). (22D) Data collected from Phenix Plus automated confocal microscope recordings obtained from spheroids in a Cal6 dye; Representative time series plots showing calcium activity phenotypes in SNSs without astrocytes (100% dopaminergic, glutamatergic, or GABAergic neurons) or with 10% astrocytes and 90% neuron (22E) Quantified peak count, amplitude, and width data along with correlation score representing the average R² value from all identified ROIs in each spheroid. In SNS dopaminergic spheroids, astrocytes reduced peak count and increased peak amplitude/width. In SNS glutamatergic spheroids, astrocytes increased peak count and reduced peak amplitude/width along with a slight reduction in synchrony. Astrocytes did not alter activity in SNSs with GABAergic neurons. (Data was run in technical replicates of n=4-6 per plate across one experiment for SNSs; For b., unpaired t-tests were used to compare SNSs with the same neuronal subtype (with vs without astrocytes), data is represented as mean±SEM, * p<0.05, ** p<0.01, *** p<0.001, ****p<0.0001.

FIGS. 23A and 23B depict the functional responses to quality control (QC) compounds in brain region-specific neural spheroids. Data collected from FLIPR recordings obtained from spheroids in a Cal6 dye (23A) Representative time series plots showing calcium activity phenotypes after treatment with control compounds targeting receptors for each neuronal cell type from VTA-like spheroids on top and PFC-like spheroids on the bottom. Left to right: DMSO (vehicle), Bicuculline (GABAAR antagonist), Muscimol (GABAAR agonist), CNQX (AMPAR antagonist), Memantine (NMDAR antagonist), SCH23390 (Dopamine 1 receptor (D1R) antagonist), and Sulpiride (D2R antagonist). (23B) Radar plots depicting multiparametric calcium activity response to each compound relative to DMSO controls in VTA-like (top) and PFC-like (bottom) spheroids. (For each compound, wells were tested at n=3-4 technical replicates over three separate experiments; For b., results are normalized to DMSO-treated control wells such that averages for DMSO-treated control wells on the radar plot equal 100%. Data from (23B) are represented as mean values for each parameter.

FIGS. 23C and 23D depict the functional responses to control compounds in brain region-specific neural spheroids throughout the 60-min recording period. (23C) Data from VTA-like spheroids (23D). Data from PFC-like spheroids (23C, 23D) Calcium activity, relative to DMSO-treated controls, during four recordings including baseline plus 1-, 30-, and 60-min after treatment with control compounds. Compounds used to target GABA_{A} receptors included agonist, muscimol, and antagonist, bicuculline. Ionotropic glutamate receptors were targeted with CNQX, AMPAR antagonist, and Memantine, NMDAR antagonist while dopamine receptors were targeted with SCH23390, D1R antagonist, and sulpiride, D2R antagonist. Data is shown for all 10 peak parameters analyzed and is represented as mean±95% confidence interval. Linear mixed model ANOVA was used to examine repeated measures fitted to a mixed model, with treatment as the between-subjects factor and recording as the within-subjects factor. Analysis results are represented in Table 5.

FIGS. 24A, 24B, and 24C depict comparisons of spheroid viability and select peak parameters from FLIPR data between wildtype and disease models. (24A) Luminescence from spheroids exposed to the 3D Cell Titer Glo (CTG) assay to measure spheroid viability (3D CTG assay: n=8-10 technical replicates, n=1 biological replicate for each disease line collected over three independent experiments. Results are analyzed with unpaired t-tests (24A, 24B) and One way ANOVA (24C) with significance set at p<0.05. Data from are represented as mean±SEM; *p<0.05, **p<0.01, ***p<0.001, ****p<0.0001.

FIGS. 25A-25I depict that clinically approved compounds to treat symptoms of Alzheimer's Disease (AD) reverse deficits caused by the incorporation of APOE4/4 GABA neurons in PFC-like spheroids to model AD. (25A, 25B) Data collected from confocal recordings (25A) Baseline phenotypic differences in PFC-like spheroids containing GABA neurons expressing APOE3/3 (Wildtype, top panel) or APOE4/4 (bottom panel) to model AD; Left: average signal across all identified ROIs, Middle: heatmap showing activity of all identified ROIs, Right: correlation matrix showing synchrony of ROIs (25B) Quantification of peak count, amplitude, width, and synchrony from confocal recordings (25C-25I) Data collected from FLIPR recordings (25C) Baseline peak count, amplitude, and width from all wells recorded with FLIPR (25D) PCA on baseline multiparametric peak data represented as a scatterplot displaying individual values (25E) Predictive labeling of genotype based on PCA data is 94% accurate using Random Forest machine learning classifier model; Data represented as a confusion matrix showing accurate and erroneous error labels for each genotype, values represented as percentages (25F) Representative time series traces 90 min after Wt and APOE4 spheroids were treated with compounds used to treat AD (25G, 25H) Peak count (25G) and peak spacing (25H) in Wt PFC-like spheroids treated with DMSO and APOE4 spheroids divided by treatment group at baseline (left) and 90 min after treatment (right); Donepezil, Memantine, and EUK-134 restore deficits in peak count (25I) Radar plots showing phenotypic data across 10 peak parameters measured 90 min after treatment with DMSO in Wt (gray) and APOE4 (red) spheroids, along with treatment with either Donepezil, Memantine, and EUK-134 (teal). (For confocal recordings, n=4-8 per group over 2 separate experiments; For FLIPR recordings, n=249 samples over 3 separate experiments. Data from (25B, 25G, 25H) represented as mean±SEM, (25B) analyzed with unpaired t-tests for each spheroid type & (25G, 25H) analyzed with One way ANOVA followed up with Dunnett's posthoc. Data from (25C) represented as violin plots with median indicated by red line; Mann-Whitney unpaired t-test used to compare medians between groups, (25I) represented as radar plots showing group averages for each peak parameter analyzed. * p<0.05, **p<0.01, ***p<0.001, ****p<0.0001).

FIGS. 26A and 26B depict the effects of compounds used to treat spheroids modeling Alzheimer's Disease on Wt PFC-like spheroids. (26A, 26B) Data collected from FLIPR recordings from spheroids incubating in Cal6 dye; Wt and APOE4 PFC-like spheroids at baseline and 90 min after treatment with either DMSO or compounds used to treat AD. At baseline, significant chances in peak count (26A) and spacing (26B) were only observed in DMSO-treated APOE4 spheroids. At 90 min, differences were observed in all groups except Rivastigmine (n=8-12 technical replicates, n=3 biological replicates collected over three independent experiments. Results are analyzed with One way ANOVA (for baseline PkCt, 90 min PkCt, baseline PkSp, 90 min PkSp, respectively: F_{(8,94)}=13.61, 28.82, 20.17, 54.6, p<0.0001) with significance set at p<0.05. Data from are represented as mean±SEM; *p<0.05, **p<0.01, ***p<0.001, ****p<0.0001.

FIGS. 27A-27I depict dopamine agonist, Ropinirole, reverses deficits induced by incorporation of mutant alpha-synuclein (SNCA A53T) dopaminergic neurons into VTA-like spheroids to model Parkinson's Disease (PD). (27A, 27B) Data collected from confocal recordings (27A) Baseline phenotypic differences in VTA-like spheroids containing wildtype (top panel) or mutant A53T dopaminergic neurons (bottom panel) to model PD; Left: average signal across all identified ROIs, Middle: heatmap showing activity of all identified ROIs, Right: correlation matrix showing synchrony of ROIs (27B) Quantification of peak count, amplitude, width, and synchrony from confocal recordings (27C-I) Data collected from FLIPR recordings (27C) Example trace showing peak detection analysis as well as quantification of baseline peak count, amplitude, and width from all wells recorded with FLIPR (27D) Example trace showing subpeak detection analysis as well as quantification of baseline peak count, amplitude, and width from all wells recorded with FLIPR (27E) PCA on baseline multiparametric peak data represented as a scatterplot displaying individual values (27F) Predictive labeling of genotype based on PCA data is 96% accurate using Random Forest machine learning classifier model; Data represented as a confusion matrix showing accurate and erroneous error labels for each genotype, values represented as percentages (27G) Representative time series traces 90 min after Wt and A53T spheroids were treated with DMSO or Ropinirole, a dopamine agonist (27H) Peak count, peak width, and subpeak count in Wt VTA-like spheroids treated with DMSO and A53T spheroids treated with Ropinirole, which restored deficits across all 3 peak measures (27I) Radar plots showing phenotypic data across 10 peak parameters measured 90 min after treatment with DMSO in Wt (gray) and A53T (red) spheroids, along with treatment with Ropinirole across 3 concentrations (teal). (For confocal recordings, n=8-10 per group over 2 separate experiments; For FLIPR recordings, n=370 samples over 3 separate experiments. Data from (27B, 27H) represented as mean±SEM, (27B) analyzed with unpaired t-tests for each spheroid type & (27H) analyzed with One way ANOVA followed up with Dunnett's posthoc. Data from (27C) represented as violin plots with median indicated by red line; Mann-Whitney unpaired t-test used to compare medians between groups, (27I) represented as radar plots showing group averages for each peak parameter analyzed. * p<0.05, **p<0.01, ***p<0.001, ****p<0.0001).

FIGS. 28A and 28B depict the effects of compounds used to treat spheroids modeling Parkinson's Disease on Wt and A53T VTA-like spheroids. (28A, 28B) Data collected from FLIPR recordings from spheroids incubating in Cal6 dye; Wt and A53T VTA-like spheroids at baseline and 90 min after treatment with either DMSO or compounds used to treat PD.

FIGS. 29A-29F show that naloxone (MOR antagonist) reverses deficits induced by chronic opioid treatment in PFC-like but not VTA-like spheroids in spheroids modeling Opioid Use Disorder. (29A, 29B) Data collected from confocal recordings (29A) Baseline phenotypic differences in PFC- and VTA-like spheroids pre-treated with either vehicle (water, top panel), chronic DAMGO (middle panel), or chronic DAMGO plus a 3-day washout to model drug withdrawal; Left: average signal across all identified ROIs, Middle: heatmap showing activity of all identified ROIs, Right: correlation matrix showing synchrony of ROIs (29B) Quantification of peak count, amplitude, width, and synchrony from confocal recordings (29C-29F) Data collected from FLIPR recordings (29C) Quantification of baseline peak count, amplitude, and width from all wells recorded with FLIPR (29D) PCA on baseline multiparametric peak data represented as a scatterplot displaying individual values in PFC-like (left) and VTA-like spheroids (right) (29E) Predictive labeling of pre-treatment group based on PCA data using Random Forest machine learning classifier model; Data represented as a confusion matrix showing accurate and erroneous error labels for each pre-treatment group, values represented as percentages (29F) Peak count and peak spacing in PFC-like spheroids; Data shows baseline differences between spheroids with vehicle vs chronic DAMGO pre-treatment (left panel), 30 min after DAMGO treatment (middle panel), and 60 min after DAMGO treatment plus 30 min after naloxone treatment; Data shows that naloxone is able to reverse deficits induced by chronic DAMGO pre-treatment and DAMGO treatment (29F) Radar plots showing phenotypic data across 10 peak parameters measured 60 min after treatments with either DMSO, DAMGO, and/or naloxone. (For confocal recordings, n=7-8 per group over 2 separate experiments; For FLIPR recordings, n=171 (PFC-like) and 135 samples (VTA-like) over 3 separate experiments. Data from (29B, 29F) represented as mean±SEM, analyzed with One way ANOVA followed up with Dunnett's posthoc. Data from (29C) represented as violin plots with median indicated by solid line; Brown-Forsythe and Welch One way ANOVA used to compare medians between groups, (29F) represented as radar plots showing group averages for each peak parameter analyzed. * p<0.05, **p<0.01, ***p<0.001, ****p<0.0001).

FIGS. 30A-30G show that functional assembloids can be made from conjoined spheroids to model neural circuitry. The spheroids were transfected with DREADDs viruses tagged with mCherry and activity can be recorded with a FLIPR at 3-weeks using a cal6 dye. (30A) Proof-of-concept study showing that activity from single neuron spheroids (SNSs) comprised of 90% neuron and 10% astrocytes can be recorded and displays similar phenotypes in spheroids transfected with stimulatory (hM3Dq) or inhibitory (hM4Di) DREADDs viruses. Top panel: vehicle control-treated spheroids expressing no DREADDs virus; Middle panel: 60-min after treatment with CNO to activate DREADDs virus and induce stimulatory activity; Bottom panel: 60-min after treatment with CNO to activate DREADDs virus and induce inhibitory activity (30B) Radar plots showing multiparametric peak alterations across 10 peak parameters for both stimulatory (light grey) and inhibitory (dark grey) DREADDs in SNSs with dopaminergic, glutamatergic, and GABAergic neurons, respectively. (30C) Representative image of assembloid where the VTA-like component expressed GCaMP6f and the PFC-like component expressed inhibitory DREADD, hM4Di (30D) Quantification of baseline activity of the assembloid before (light grey/second bar) and after (dark gray/third bar) CNO was added to media to activate the inhibitory DREADD (30E) Representative image of assembloid where the PFC-like component expressed GCaMP6f and the VTA-like component expressed inhibitory DREADD, hM4Di (30F) Quantification of baseline activity of the assembloid before (light grey/second bar) and after (dark gray/third bar) CNO was added to media to activate the inhibitory DREADD.

### DETAILED DESCRIPTION

Before the subject disclosure is further described, it is to be understood that the disclosure is not limited to the particular embodiments of the disclosure described below, as variations of the particular embodiments may be made and still fall within the scope of the appended claims. It is also to be understood that the terminology employed is for the purpose of describing particular embodiments and is not intended to be limiting. Instead, the scope of the present disclosure will be established by the appended claims.

Throughout the present specification and the accompanying claims, the words “comprise” and “include” and variations such as “comprises”, “comprising”, “includes” and “including” are to be interpreted inclusively. That is, these words are intended to convey the possible inclusion of other elements or integers not specifically recited, where the context allows. The articles “a” and “an” are used herein to refer to one or to more than one (i.e., to one or at least one) of the grammatical object of the article. By way of example, “an element” may mean one element or more than one element. Unless defined otherwise, all technical and scientific terms used herein have the same meaning as commonly understood to one of ordinary skill in the art to which this disclosure belongs.

The term “about,” when modifying any amount, refers to the variation in that amount typically encountered by one of skill in the art, i.e., in the field of stem cell and spheroid formation and differentiation. For example, the term “about” refers to the normal variation encountered in measurements for a given analytical technique, both within and between batches or samples. Thus, the term about can include variation of 1-10% of the measured amount or value, such as +/−1%, 2%, 3%, 4%, 5%, 6%, 7%, 8%, 9% or 10% variation. The amounts disclosed herein include equivalents to those amounts, including amounts modified or not modified by the term “about.”

“AdaBoost,” as used herein, refers broadly to a bagging method that iteratively fits CARTs re-weighting observations by the errors made at the previous iteration.

“Adherent culture,” as used herein refers broadly to a cell culture system whereby cells are cultured on a solid surface, which allows cells to proliferate and stabilize in culture.

“Classifier,” as used herein, refers broadly to a machine learning algorithm such as support vector machine(s), AdaBoost classifier(s), penalized logistic regression, elastic nets, regression tree system(s), gradient tree boosting system(s), naive Bayes classifier(s), neural nets, Bayesian neural nets, k-nearest neighbor classifier(s), Deep Learning systems, and random forests. This invention contemplates methods using any of the listed classifiers, as well as use of more than one of the classifiers in combination.

“Classification and Regression Trees (CART),” as used herein, refers broadly to a method to create decision trees based on recursively partitioning a data space so as to optimize some metric, usually model performance.

“Classification system,” as used herein, refers broadly to a machine learning system executing at least one classifier.

“Elastic Net,” as used herein, refers broadly to a method for performing linear regression with a constraint comprised of a linear combination of the L1 norm and L2 norm of the vector of regression coefficients.

“False Positive (FP)” and “False Positive Identification,” as used herein, refers broadly to an error in which the algorithm test result indicates the presence of a disease when the disease is actually absent.

“False Negative (FN),” as used herein, refers broadly to an error in which the algorithm test result indicates the absence of a disease when the disease is actually present.

“LASSO,” as used herein, refers broadly to a method for performing linear regression with a constraint on the L1 norm of the vector of regression coefficients.

“Neural cells,” as used herein, refers broadly to cells originating in the central nervous system. Neural cells include, but are not limited to, astrocytes, microglia, oligodendrocytes, and neurons.

“Neural Net,” as used herein, refers broadly to a classification method that chains together perceptron-like objects to create a classifier.

“Neurons,” as used herein, refers broadly to an electrically excitable cell that communicates with other cells via synapses. Also referred to as “nerve cells.”

“Performance score,” as used herein, refers broadly to the distances between predicted values and actual values in the training data. This is expressed as a number between 0-100%, with higher values indicating the predicted value is closer to the real value. Typically, a higher score means the model performs better.

“Plated” and “plating,” as used herein, refers broadly to any process that allows cells to be grown in a suspension or adherent culture.

“Random Forest,” as used herein, refers broadly to a bagging method that fits CARTs based on samples from the dataset that the model is trained on.

“Standard of Deviation (SD),” as used herein, is the spread in individual data points (i.e., in a replicate group) to reflect the uncertainty of a single measurement.

“Subset,” as used herein, refer broadly to a proper subset and “superset” is a proper superset.

“Suspension,” as used herein, refers broadly to a cell culture system whereby cells are grown in the media and do not adhere to any substrate.

“Spheroid” as used herein refers broadly to an artificial construct comprising a plurality of neurons, and optionally glial cells that have functional properties substantially similar to regions in the brain.

“Training Set,” as used herein, is the set of samples that are used to train and develop a machine learning system, such as an algorithm used in the method and systems described herein.

“Validation Set,” as used herein, refers broadly to the set of samples that are blinded and used to confirm the functionality of the algorithm used in the method and systems described herein. This is also known as the Blind Set.

### Brain Region Specific Spheroids

The present disclosure relates to brain region-specific spheroids, methods of making and methods of using the same.

The spheroids described herein are more high-throughput compatible than other complex in vitro models, e.g., organoids, bioengineered tissue, or organ-on-a-chip, while still retaining physiological complexity. The spheroids described herein may be designed to model specific brain regions, e.g., prefrontal cortex (PFC), nucleus accumbens, amygdala, hippocampus, ventral tegmental area (VTA), by combining differentiated neural cells and/or neural associated cells in specific amounts. The spheroids described herein are artificial constructs which exhibit physiological properties substantially similar to different brain regions.

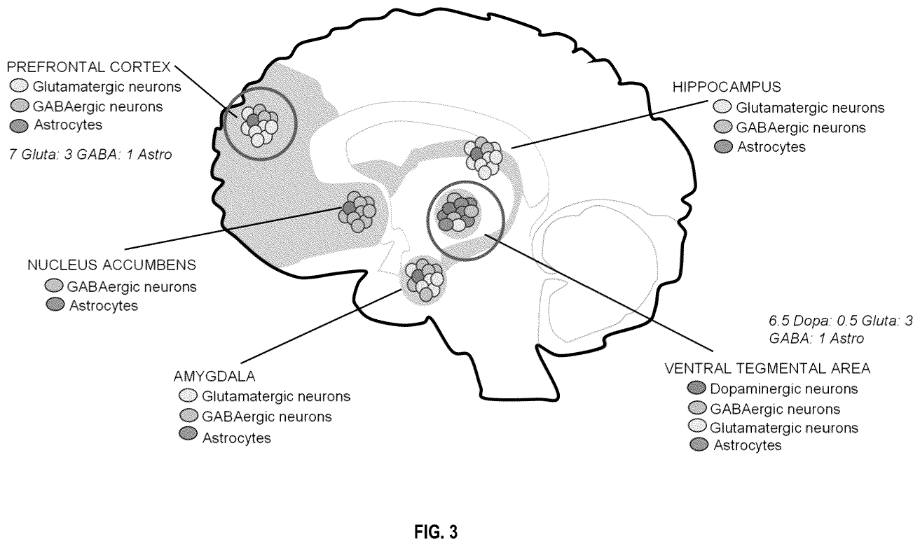

The spheroids may form brain-specific-region like architecture that models brain regions. Example brain regions include, but are not limited to, prefrontal cortex (PFC), nucleus accumbens, amygdala, hippocampus, and ventral tegmental area (VTA). These spheroids are referred to as prefrontal cortex (PFC)-like, nucleus accumbens-like, amygdala-like, hippocampus-like, and ventral tegmental area (VTA)-like. In some embodiments, the brain specific-region spheroids comprise combinations of neuronal and glial cells as shown in FIG. 3.

PFC-like spheroids may comprise PFC-like cells. In some embodiments, PFC-like spheroids may comprise combinations of Glutamatergic neurons, GABAergic neurons, and astrocytes. In another embodiment, PCF-like spheroids are characterized by the presence of at least one or more of the following features: a ventromedial cortex and a lateral cortex.

VTA-like spheroids may comprise VTA-like cells. In some embodiments, the VTA-like spheroids comprise combinations of dopaminergic neurons, GABAergic neurons, glutamatergic neurons, and astrocytes. In another embodiment, VTA-like spheroids are characterized by the presence of at least one or more of the following features: a paranigral nucleus (PN), a parabrachial pigmented area (PBP), a parafasciculus retroflexus area (PFR), and a rostromedial tegmental nucleus (RMTg).

The nucleus accumbens-like spheroids may comprise nucleus accumbens-like cells. In some embodiments, nucleus accumbens-like spheroids comprise combinations of GABAergic neurons and astrocytes. In another embodiment, nucleus accumbens-like spheroids are characterized by the presence of at least one or more of the following features: medium spiny neurons and fast spiking interneurons.

The hippocampus-like spheroids may comprise hippocampus-like cells. In some embodiments, hippocampus-like spheroids comprise combinations glutamatergic neurons, GABAergic neurons, and astrocytes.

The present disclosure also provides an in vitro model for opioid use disorder (OUD).

Easier-to-assemble 3D culture models, like spheroids, can be both produced and studied in high-throughput formats, but they must have appropriate cell complexity and physiological function.

The brain-region specific spheroids described herein have a distinct advantage of being more compatible for high-throughput formats, but a further advantage is their ability to be generated in a carefully controlled fashion, with modular cellular components. This advantage allows for the incorporation not only of different desired ratios of varying neural and/or neural associated cell subtypes, but also for the incorporation of diseased versus healthy cellular types, for example, or diseased astrocytes into an otherwise healthy neuronal spheroid. This modular approach also allows for the incorporation of genetically modified subpopulations, e.g. dopaminergic neurons with cell-type specific promoter-driver reporters such as genetically encoded calcium indicators, (GECIs, such as GCaMP) to selectively monitor activity or the use of designer receptors exclusively activated by designer drugs (DREADDS) and optogenetics to selectively manipulate activity of a particular neuronal subtype. The protocol disclosed herein produces functional brain models in just 3 weeks, with tailored brain region specific cell composition to mimic areas of the brain. These designer spheroids are reproducible in size and function, so they are amenable to HTS. This process should be amenable to brain models of different species and creating neuronal circuits in which spheroids from different regions of the brain are connected.

Notably, prior attempts failed to form uniform and functional spheroids from fully matured neuronal cells. Hasan et al. “Neural layer self-assembly in geometrically confined rat and human 3D cultures.” Biofabrication 2019; 11:045011. Prior cultures have yielded non-uniform, discontinuous, and significantly non-proliferating cells.

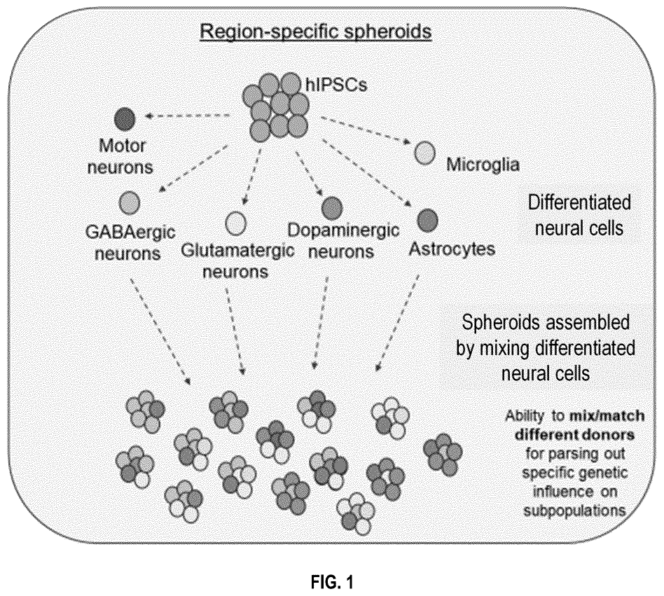

The method of assembly of the spheroids described herein may comprise (a) obtaining differentiated neural cells and/or neural associated cells; (b) admixing the differentiated neural and/or neural associated cells, (c) culturing said admixed neural and/or neural associated cells for a period of time in medium sufficient to allow for the formation of spheroids. The neural cells may be neurons, glial cells, or a combination thereof. FIG. 1.

The inventors surprisingly discovered that assembling the spheroids from differentiated neurons produced a spheroid with superior properties. In contrast to other methods, the spheroids described herein are assembled from differentiated neurons.

Methods for assembly of brain region-specific spheroids including but not limited to the ventral tegmental area (VTA), prefrontal cortex (PFC), nucleus accumbens, amygdala, hippocampus, somatomotor cortex, somatosensory cortex, parietal lobe, occipital lobe, cerebellum, and temporal lobe are described herein. FIG. 3. The brain region-specific spheroids described herein may exhibit electrophysiological properties, calcium activity profile, neurotransmitter release, or a combination thereof, substantially similar to cells from a defined brain region selected from the ventral tegmental area (VTA), prefrontal cortex (PFC), nucleus accumbens, amygdala, hippocampus, somatomotor cortex, somatosensory cortex, parietal lobe, occipital lobe, cerebellum, and temporal lobe.

The spheroids can be used in methods for evaluating the effects of an agent on brain tissue. The methods comprise forming spheroids in culture media. The test agent is a small molecule drug or other biomolecule or compound. The methods further comprise assaying/assessing the effects of the test agent on the spheroid. Characteristics that may be assessed include, for example, cell growth, proliferation, cytotoxicity, and/or differentiation, change in biomarker expression, and/or change in axonal growth rate and/or pattern. Other characteristics may be assessed.

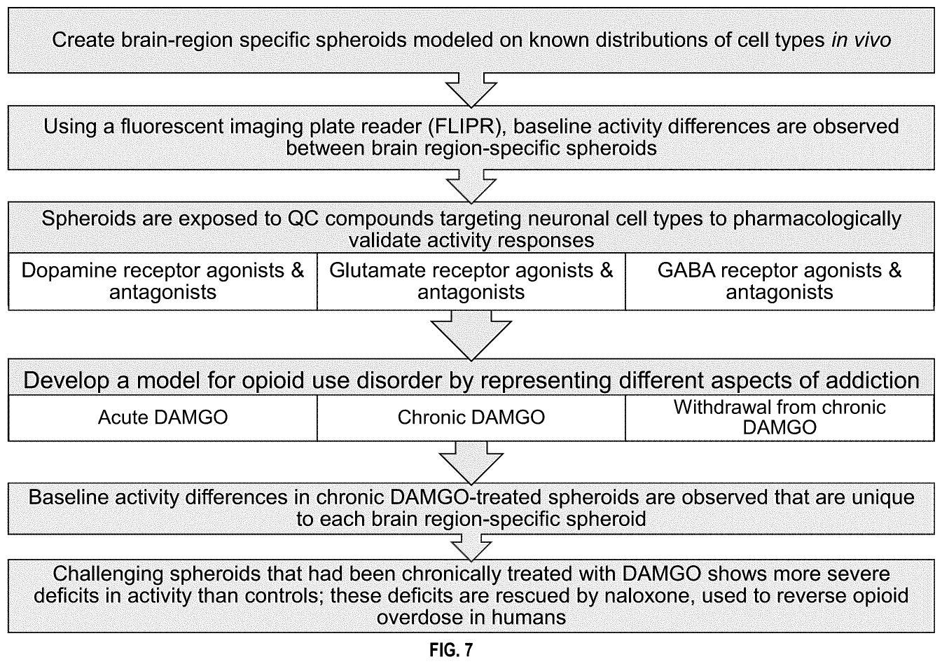

The spheroids may be used as an in vitro model for opioid use disorder (OUD). Animal models of addiction can recapitulate distinct phases of addiction (acute drug exposure, drug dependence, craving, withdrawal) based on length of drug exposure. Scofield 2016: The Nucleus Accumbens: Mechanisms of Addiction across Drug Classes Reflect the Importance of Glutamate Homeostasis. Pharmacol Rev. doi: 10.1124/pr.116.012484. Chronic recreational drug use can be modeled through chronic drug administration while craving and withdrawal periods can be modeled by exposing an animal to a period of forced abstinence. Furthermore, relapse behaviors can be modeled by exposing the animal to a challenge dose of drug after a period of forced abstinence. In this way, it is possible to similarly model various aspects of drug dependence in spheroids by manipulating the exposure time to opioids, exposing them to a period of forced abstinence (e.g., “withdrawal) and challenging with opioids again after this period of abstinence. Neural activity changes observed in VTA- and PFC-like spheroids match what is shown during these various phases mentioned above in the VTA and PFC of animal models as well as what has been shown in human studies with fMRI. FIG. 7.

A major hurdle facing therapeutics development for neurological diseases is the lack of predictable cellular assay platforms for disease modeling and drug screening. Cellular neural models range from two-dimensional (2D) cellular monolayers to 3D organoids, both of which lack functional reproducibility on high-throughput (HT) assay testing platforms. Neural spheroids are 3D cell aggregates that embody the robustness of 2D models and physiological complexity of 3D organoids but contain uncontrolled neuronal subtype populations, hinder their functional reproducibility. To address this, the inventors developed a HT functional assay platform where neural spheroids were made with matured, differentiated human induced pluripotent stem cell (hiPSC)-derived neurons and astrocytes combined in controlled cell-type compositions reflecting that of specific brain regions described herein. The inventors developed spheroids modeled after the cellular composition of the human prefrontal cortex and ventral tegmental area (PFC- and VTA-like spheroids, respectively), and functional readouts were measured by fluctuations in calcium fluorescence. Disease models were developed for Alzheimer's and Parkinson's Disease (AD and PD) along with Opioid Use Disorder (OUD), and a machine learning classifier model showed that the AD and PD models displayed baseline deficits that were highly predictable. Furthermore, phenotypic deficits in diseased spheroids were reversed with treatments clinically approved to treat each disease in humans. In addition, these spheroids can be used to create neural circuit-specific assembloids, and chemogenetic approaches can be used to manipulate circuit activity. Brain region-specific neural spheroids as a robust functional assay platform for neurological disease modeling and drug screening are described herein.

Therapeutic development for neurological diseases is hindered by several factors including a lack of predictable in vitro cellular assays, low-throughput animal model studies, cost and complexity of clinical trials, along with inadequate neurological disease modeling (DiMasi et al., 2016; Wong et al., 2019). This is demonstrated by the fact that less than 10% of treatments for neurological diseases in clinical trials are approved by the Federal Drug Administration each year (DiMasi et al., 2016; Hay et al., 2014; Wong et al., 2019). With diagnoses of neurodegenerative diseases and addiction on the rise over the last few decades, it is critical to develop ways to improve drug discovery for neurological diseases (Hawk et al., 2021; Olfson et al., 2021; Rehm et al., 2019). The inventors addressed this need in the art by exploring a general approach that seemed to be a promising field of experimentation, where the prior art gave only general guidance as to the particular form of the invention described herein or how to achieve it.

Three-dimensional (3D) cellular neural models derived from induced pluripotent stem cells (iPSCs), including organoids and spheroids, have gained traction as a tissue platform for neurological drug discovery over traditional 2D cellular models. While 2D neural models can be a robust platform compatible with high-throughput screening (HTS) study designs, 3D neural tissue models are better able to recapitulate in vivo neurophysiology. For instance, studies have shown that 2D cellular monolayers display reduced gene expression for markers of neuronal function and shorter neurite outgrowth compared to 3D tissue models. This is accompanied by reductions in population neuronal activity along with greater intra-plate variability among 2D cellular models. However, while 3D organoids acquire greater cellular complexity and some brain-like organization, their complexity can hinder their ability to be implemented in HTS assay platforms. Organoids can suffer from batch-to-batch variation in both size and cell composition heterogeneity, limited differentiation of neuronal cell types, and lengthy differentiation and maturation times.

Spheroids are 3D cell aggregates generated by cellular self-assembly, giving them the ability to achieve the robustness of 2D cellular models while maintaining the complexity of 3D organoids. Traditionally, neural spheroids are derived from neural stem cells (NSCs) that differentiate in culture, and while these are more readily adaptable for HTS than 2D cellular and 3D organoid models, they are primarily limited to cortical neurons, limiting their cell type complexity to what cell types can be co-differentiated together. Furthermore, the ratios of neuronal subtypes that NSCs differentiate into can vary from spheroid to spheroid, limiting the ability to model specific subregions of cortex or other brain regions with more diverse neuronal subtype populations such as dopaminergic neurons. In order to improve drug discovery for neurological diseases, there exists a need for a HTS-compatible 3D tissue model system that has more control over the neural cell type composition to enhance both functional reproducibility and biological relevance.

To address these challenges, the inventors developed HTS-compatible neural spheroid system with high inter- and intra-batch reproducibility that are customizable to incorporate different neural cells at desired ratios in a 384-well plate format. This method combines differentiated human induced pluripotent stem cell (hiPSC)-derived neurons and astrocytes in controlled cell-type compositions reflecting what is found in specific regions of the human brain. Functional readouts were measured through intracellular calcium oscillations, which have been shown to be highly correlated with the electrophysiological properties of neurons. Fluctuations in calcium fluorescence were recorded from spheroids in a calcium dye (Cal6) or expressing a genetically encoded calcium indicator (GCaMP6f) using both an automated confocal for image-based recordings and a fluorescent imaging plate reader (FLIPR) that records population spheroid activity from all wells simultaneously to demonstrate HTS-compatibility.

With the intention of developing disease models for Alzheimer's Disease (AD), Parkinson's Disease (PD), and Opioid Use Disorder (OUD), the inventors created spheroids modeling two brain regions with significant overlap between these three diseases: the ventral tegmental area (VTA) and prefrontal cortex (PFC). Two spheroid types were developed (termed VTA-like and PFC-like spheroids) based on the cell-type composition of these regions as indicated from postmortem human brain studies showing that the VTA contains roughly 65% dopaminergic neurons, 5% glutamatergic neurons, and 30% GABAergic neurons while the PFC contains roughly 70% glutamatergic neurons and 30% GABAergic neurons. Given that AD is characterized by neurodegeneration in neocortical brain areas while PD is caused by cell death in dopaminergic neurons, the AD model described herein was developed in PFC-like spheroids while the PD model was developed in VTA-like spheroids. Additionally, our spheroids modeling OUD were tested with both spheroid types given that OUD involves dysregulated dopamine release from the VTA to the PFC, which further alters PFC glutamatergic signaling.

The AD and PD models were developed by incorporating genetically engineered cell lines with mutations commonly associated with each disease, while OUD was modeled by chronic pre-treatment with DAMGO, a mu opioid receptor (MOR) agonist. The strongest genetic risk factor for AD occurs in people carrying two copies of the is the apolipoprotein E4 (APOE4/4) allele and, as such, the inventors incorporated mutant GABA neurons expressing APOE4/4 into PFC-like spheroids. Battista et al. Curr Alzheimer Res. (2016) 13(11): 1200-1207. Mutations in the alpha-synuclein (SNCA) gene have been associated with the development of PD and therefore, the inventors incorporated dopaminergic neurons expressing mutant A53T SNCA into the VTA-like spheroids described herein given that it is associated with early onset familial PD. Dorszewska et al. Neural Reqen Res. (2022) 16(7): 1383-1391. Among all three disease models, phenotypic deficits were observed compared to “wildtype”, or healthy, control spheroids. A random forest model machine learning classifier model was used to quantify predictability of labeling disease phenotype and showed high accuracy for both the AD and PD models (>94%). Furthermore, clinically approved treatments for each disease were used to treat spheroids, and a reversal of deficits was observed among all three disease phenotypes.

The inventors further assessed whether neural circuit-specific modeling could be used with these spheroids and created functional assembloids using VTA- and PFC-like spheroids. Assembloids are fused spheroids in which neurite extensions between two aggregated spheroids form functional networks intended to mimic the long-range circuitry of the brain. The inventors established a protocol to infect spheroids with either GCaMP6f, for calcium activity measurement, or designer receptors exclusively activated by designer drugs (DREADDs) viruses, for chemogenetic cell silencing or stimulation, prior to fusing assembloids. Here the inventors found that the altered phenotypes in assembloids versus individual spheroids, but that silencing the DREADDs-expressing component of the assembloid can revert the phenotype closer to how it was as a single spheroid, indicative of the plasticity of these circuits. The brain region-specific neural spheroids described herein may be used for disease and neurocircuitry modeling, and for use as HTS-compatible drug screening platforms.

### Neural Cells

Neural cells comprise neurons, glial cells, and combinations thereof. The neurons may be derived from pluripotent stem cells including but not limited to embryonic stem cells (ES) cells, embryonic germ (EG) cells, induced pluripotent cells (iPSC), and combinations thereof. The pluripotent stem cells may be human induced pluripotent stem (iPS) cells. The pluripotent stem cells may be iPS cells derived from a mouse, rat, primate, ape, sheep, or monkey. The iPS cells may be derived from a healthy donor (e.g., a healthy human donor). The iPS cells may be derived from a subject with a disease (e.g., a human with a disease, such as a genetic disease). The disease may be a neurological or neurodegenerative disease. The disease may be, without limitation, autism, epilepsy, Huntington's Disease, schizophrenia, ADHD, ALS, or a bipolar disorder.

The neural cells, including iPSC derived cells, may comprise astrocytes, motor neurons, dopaminergic neurons (DopaNeurons), GABAergic neurons (GABAneurons), glutamatergic (GlutaNeurons), glia, pericytes or endothelial cells.

The neural cells may comprise neurons, glia, and combinations thereof. The glia may be astrocytes, microglia, oligodendrocytes, and combinations thereof. The neurons may be afferent neurons, efferent neurons, interneurons, and combinations thereof. The neurons may be sensory neurons, motor neurons, interneurons, and combinations thereof. The neurons may be unipolar, bipolar, pseudounipolar, multipolar, and combinations thereof. The neurons may be excitatory neurons, inhibitory neurons, and combinations thereof. The neurons may be GABAergic neurons, glutamatergic neurons, dopaminergic neurons, cholinergic neurons, serotonergic neurons, and combinations thereof.

Cells derived from pluripotent cells may be purchased commercially. For example, iCell® Neurons, iCell® DopaNeurons, and iCell® Astrocytes are derived from human iPS cells and may be purchased from Fujifilm Cellular Dynamics International (Madison, Wisconsin).

The amount of glial cells in a spheroid may be between 1% and 100% by total number of cells. The spheroid may comprise between about 1% and 20% glial cells by total number of cells, or between about 5% and 25% glial cells by total number of cells, or between about 10% and 75% glial cells by total number of cells. For example, the spheroid may comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 91%, 92%, 93%, 94%, 95%, 96%, 97%, 98%, 99%, or 100% glial cells by total number of cells.

The amount of neurons may be between 1% and 100% by total number of cells. The spheroid may comprise between about 10% and 40% neurons by total number of cells, or between about 50% and 75% neurons by total number of cells, or between about 50% and 95% neurons by total number of cells. For example, the spheroid may comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 91%, 92%, 93%, 94%, 95%, 96%, 97%, 98%, 99%, or 100% neurons by total number of cells.

The amount of GABAergic neurons may be between 1% and 100% by total number of cells. The spheroid may comprise between about 10% and 40% GABAergic neurons by total number of cells, or between about 50% and 95% GABAergic neurons by total number of cells, or between about 15% and 75% GABAergic neurons by total number of cells. For example, the spheroid may comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 91%, 92%, 93%, 94%, 95%, 96%, 97%, 98%, 99%, or 100% GABAergic neurons by total number of cells.

The amount of glutamatergic neurons may be between 1% and 100% by total number of cells. The spheroid may comprise between about 10% and 40% glutamatergic neurons by total number of cells, or between about 50% and 75% glutamatergic neurons by total number of cells, or between about 50% and 95% glutamatergic neurons by total number of cells. For example, the spheroid may comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 91%, 92%, 93%, 94%, 95%, 96%, 97%, 98%, 99%, or 100% glutamatergic neurons by total number of cells.

The amount of dopaminergic neurons may be between 1% and 100% by total number of cells. The spheroid may comprise between about 10% and 40% dopaminergic neurons by total number of cells, or between about 50% and 75% dopaminergic neurons by total number of cells, or between about 50% and 95% dopaminergic neurons by total number of cells. For example, the spheroid may comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 91%, 92%, 93%, 94%, 95%, 96%, 97%, 98%, 99%, or 100% dopaminergic neurons by total number of cells.

The amount of GABAergic neurons may be between 1% and 100% by total number of neurons in the spheroid. The spheroid may comprise between about 10% and 40% GABAergic neurons by total number of neurons in the spheroid, or between about 50% and 95% GABAergic neurons by total number of neurons in the spheroid, or between about 15% and 75% GABAergic neurons by total number of neurons in the spheroid. For example, the spheroid may comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 91%, 92%, 93%, 94%, 95%, 96%, 97%, 98%, 99%, or 100% GABAergic neurons by total number of neurons in the spheroid.

The amount of glutamatergic neurons may be between 1% and 100% by total number of neurons in the spheroid. The spheroid may comprise between about 10% and 40% glutamatergic neurons by total number of neurons in the spheroid, or between about 50% and 75% glutamatergic neurons by total number of neurons in the spheroid, or between about 50% and 95% glutamatergic neurons by total number of neurons in the spheroid. For example, the spheroid may comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 91%, 92%, 93%, 94%, 95%, 96%, 97%, 98%, 99%, or 100% glutamatergic neurons by total number of neurons in the spheroid

The amount of dopaminergic neurons may be between 1% and 100% by total number of neurons in the spheroid. The spheroid may comprise between about 10% and 40% dopaminergic neurons by total number of neurons in the spheroid, or between about 50% and 75% dopaminergic neurons by total number of neurons in the spheroid, or between about 50% and 95% dopaminergic neurons by total number of neurons in the spheroid. For example, the spheroid may comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 91%, 92%, 93%, 94%, 95%, 96%, 97%, 98%, 99%, or 100% dopaminergic neurons by total number of neurons in the spheroid.

The neurons used to assemble the spheroids described herein may be differentiated.

The spheroid may further comprise endothelial cells. The amount of endothelial cells may be between 1% and 100% by total number of cells. The spheroid may comprise between about 10% and 40% endothelial cells by total number of cells, or between about 50% and 75% endothelial cells by total number of cells, or between about 50% and 95% endothelial cells by total number of cells. For example, the spheroid may comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 91%, 92%, 93%, 94%, 95%, 96%, 97%, 98%, 99%, or 100% endothelial cells by total number of cells.

The spheroid may further comprise pericytes. The amount of pericytes may be between 1% and 100% by total number of cells. The spheroid may comprise between about 10% and 40% pericytes by total number of cells, or between about 50% and 75% pericytes by total number of cells, or between about 50% and 95% pericytes by total number of cells. For example, the spheroid may comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 91%, 92%, 93%, 94%, 95%, 96%, 97%, 98%, 99%, or 100% pericytes by total number of cells.

The spheroids described herein form synapses capable of coordinated firing.

The spheroids described herein exhibit electrophysiological properties, calcium activity profile, neurotransmitter release, or a combination thereof, substantially similar to cells from a defined brain region selected from the ventral tegmental area (VTA), prefrontal cortex (PFC), nucleus accumbens, amygdala, hippocampus, somatomotor cortex, somatosensory cortex, parietal lobe, occipital lobe, cerebellum, and temporal lobe.

The spheroids described herein exhibit electrophysiological properties. The spheroid may exhibit electrophysiological properties substantially similar to cells in the ventral tegmental area (VTA). The spheroid may exhibit electrophysiological properties substantially similar to cells in the prefrontal cortex (PFC). The spheroid may exhibit electrophysiological properties substantially similar to cells in the nucleus accumbens. The spheroid may exhibit electrophysiological properties substantially similar to cells in the amygdala. The spheroid may exhibit electrophysiological properties substantially similar to cells in the hippocampus.

The spheroids described herein exhibit a calcium activity profile. The spheroid may exhibit calcium activity profiles substantially similar to cells in the ventral tegmental area (VTA). The spheroid may exhibit calcium activity profiles substantially similar to cells in the prefrontal cortex (PFC). The spheroid may exhibit calcium activity profiles substantially similar to cells in the nucleus accumbens. The spheroid may exhibit calcium activity profiles substantially similar to cells in the amygdala. The spheroid may exhibit calcium activity profiles substantially similar to cells in the hippocampus.

The spheroids described herein exhibit neurotransmitter release. The spheroid may exhibit neurotransmitter release substantially similar to cells in the ventral tegmental area (VTA). The spheroid may exhibit neurotransmitter release substantially similar to cells in the prefrontal cortex (PFC). The spheroid may exhibit neurotransmitter release substantially similar to cells in the nucleus accumbens. The spheroid may exhibit neurotransmitter release substantially similar to cells in the amygdala. The spheroid may exhibit neurotransmitter release substantially similar to cells in the hippocampus.

### Media and Culture Conditions

The neural cells, optionally differentiated iPSC derived neurons and glia, may be cultured in media. Non-limiting example of media include Neuronbasal Medium™, Neurobasal™—A Medium, Neurobasal™—B Medium and BrainPhys. The formation medium, the media used during the formation of the spheroids, may comprise neural basal media A, neural basal medium B or a combination thereof. The ratio of neural basal medium B to neural basal medium A may be 10:1, 9:1, 8:1, 7:1, 6:1, 5:1, 4:1, 3:1, 2:1, or 1:1. The ratio may be 1:10, 1:9, 1:8, 1:6, 1:5, 1:4, 1:3, or 1:2. The ratio may be 3:2, 3:4, 3:5, 3:7, 3:8, or 3:10. Methods for culturing iPSC neurons in media are described in the art. Neuronal Cell Culture: Methods and Protocols Amini & White (Eds) (2013) Humana Press; Human Embryonic Stem Cell Protocols 3^{rd} Ed. Turkesen (Ed.) (2016) Humana Press.

The spheroids are cultured in media. Media refers to a chemically defined liquid in which neurons are maintained. Non-limiting example of media include Neuron Basal Medium, Neurobasal Medium A, Neurobasal Medium B, and BrainPhys Medium. The formation medium comprises neural basal media A, neural basal medium B or a combination thereof. The ratio of neural basal media B to neural basal media A comprises 10:1, 9:1, 8:1, 7:1, 6:1, 5:1, 4:1, 3:1, 2:1, or 1:1. The ratio 1:10, 1:9, 1:8, 1:6, 1:5, 1:4, 1:3, or 1:2. The ratio is 3:2, 3:4, 3:5, 3:7, 3:8, or 3:10.

The cells can be seeded at any density unto the solid surface. The cell density of the seeded cells may be adjusted depending on a variety of factors, including but limited to the use of adherent or suspension cultures and cell culture medium and conditions. Examples of cell culture densities include, but are not limited to, 50 cells/ul, 100 cells/ul, 150 cells/μl, 200 cells/ul, 250 cells/μl, 300 cells/μl, 350 cells/μl, 400 cells/μl, 450 cells/μl, 500 cells/μl, 550 cells/μl, 600 cells/μl, 650 cells/μl, 700 cells/μl, 750 cells/μl, 800 cells/μl, 850 cells/μl, 900 cells/μl, 950 cells/μl, 1,000 cells/μl.

The cells may be cultured for between about 1 and 21 days as they form a spheroid. The cells may form a spheroid within 21 days of being admixed together.

The spheroids are cultured for extended periods of time, for up to about 15 days, up to about 30 days, or up to about 40 days. The spheroids may be cultured for about at least 1 week, at least 3 weeks, or at least 6 weeks in suspension. The spheroids are cultured for 2-6 week, 2-4 weeks, or 4-6 weeks. Longer culture times are contemplated herein.

The spheroids described herein can be between about 300-350 mm in diameter after the maturation process, have a homogenous spatial distribution of neurons and astrocytes (e.g., exhibit MAP and GFAP staining) and lack a necrotic core (e.g., as confirmed by, for example, nuclear staining). The spheroids described herein can be about 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, or 350 mm in diameter after the maturation process. The spheroids described herein can be between about 350 mm in diameter after the maturation process, have a homogenous spatial distribution of neurons and astrocytes (e.g., exhibit MAP and GFAP staining) and lack a necrotic core (e.g., as confirmed by, for example, nuclear staining).

A method of making a spheroid described herein may comprise (a) obtaining neurons, optionally differentiated neurons; (b) admixing the neurons; and (c) culturing under conditions to form a spheroid. The method may further comprise adding glial cells, optionally astrocytes.

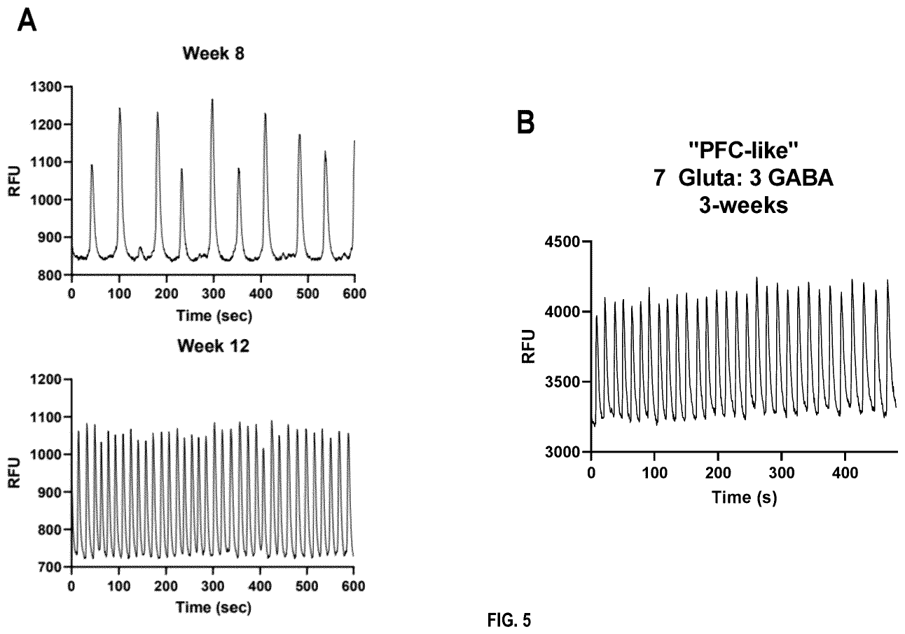

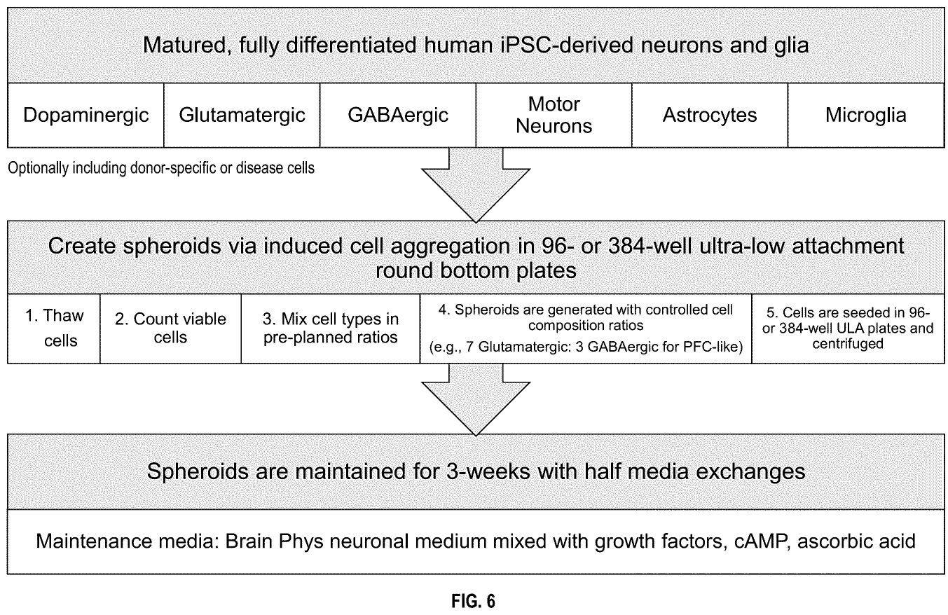

A method of making the spheroid described herein may comprise (a) obtaining neural cells comprising neurons, glia, and combinations thereof, optionally wherein the neural cells are matured, fully differentiated neural cells; (b) combining the neural cells; (c) providing agitation to the mixture of neural cells; (d) centrifuging the neural cells; (e) re-suspending the neural cells after centrifugation; (f) plating the neural cells in a vessel; and (f) culturing the neural cells under conditions to form a spheroid. FIG. 6. The neurons and/or glial may be obtained from induced pluripotent stem cells (iPSCs), optionally human iPSCs.

### Spheroid Neural Cell Composition

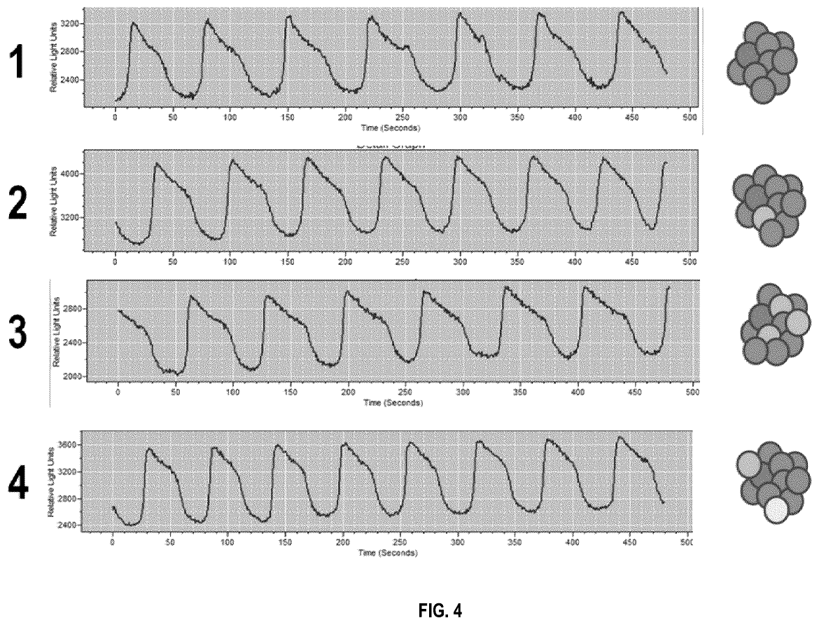

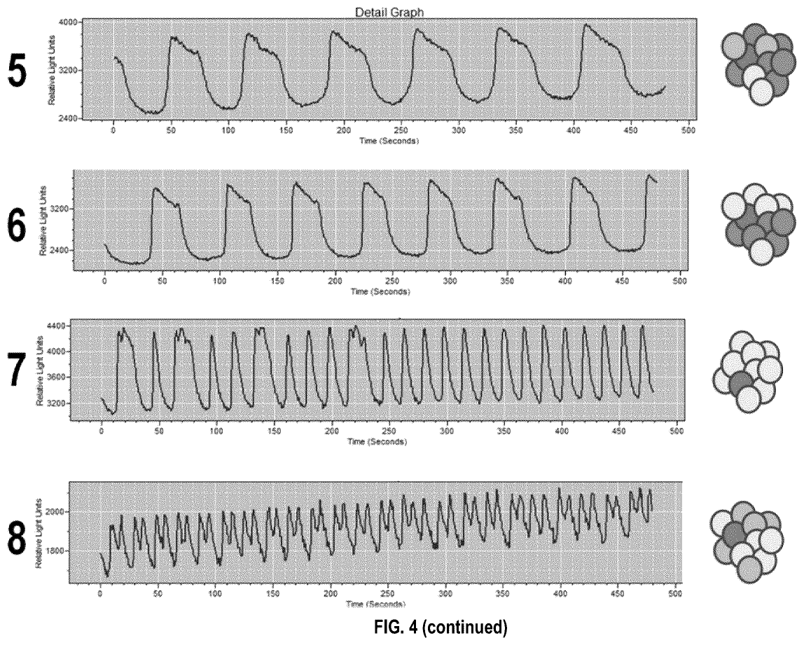

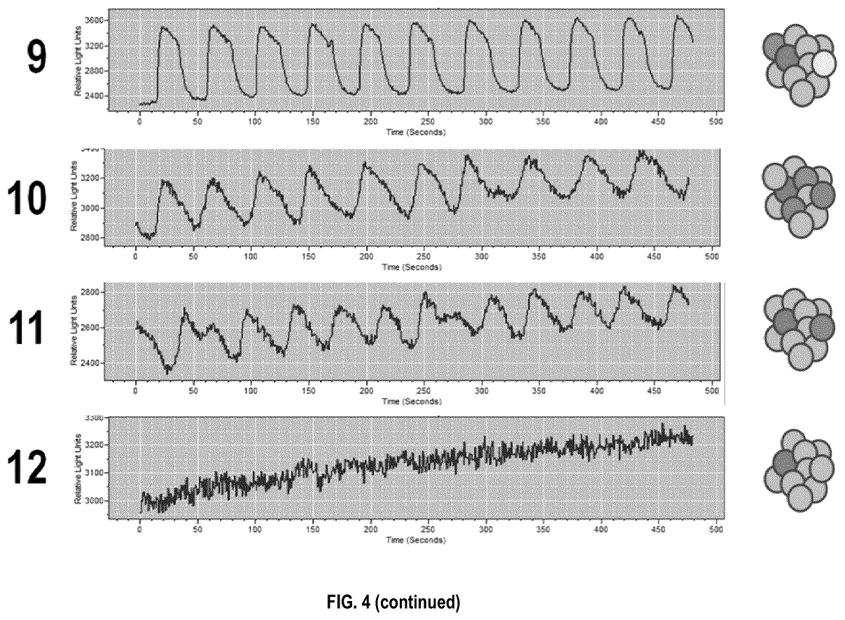

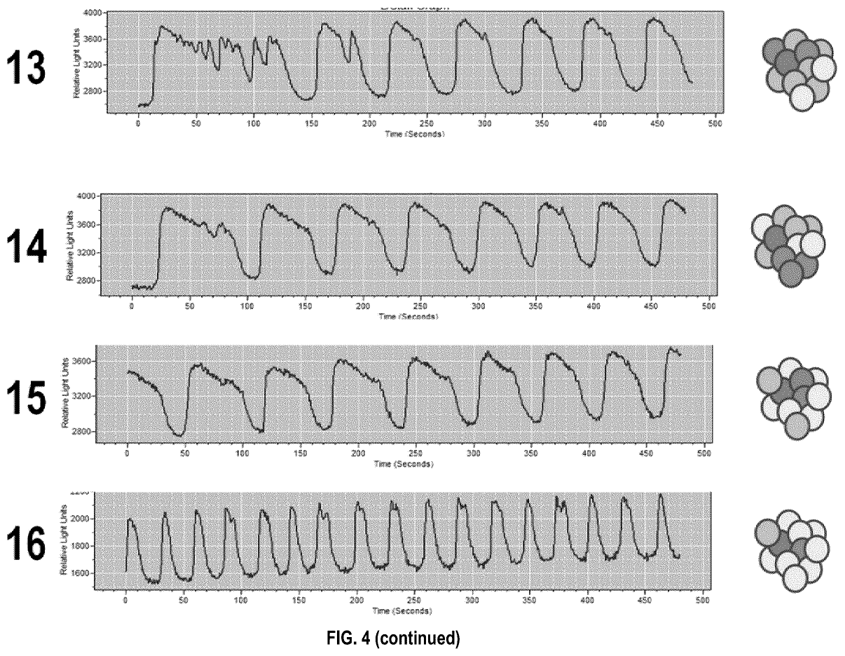

The combination of specific amounts of different neural cells allows for the formation of brain specific-region spheroids. For example, the spheroid may comprise 100% dopaminergic neurons, 100% GABAergic neurons, or 100% glutamatergic neurons by total amount of cells. The spheroids described herein are artificial constructs of neural cells that replicate the physiology, including electrophysiology, of areas of the mammalian brain. FIGS. 4-5. The spheroids described herein are engineered to comprise neurons and/or glial cells and have properties substantially similar to those of areas of the mammalian brain. FIGS. 4-5. The spheroids described herein are fabricated using the methods described herein.

The spheroid may comprise a combination of dopaminergic neurons and GABAergic neurons. Approximately equal amounts of the dopaminergic neuron and GABAergic neuron may be present in the composition. Alternatively, the composition may comprise more dopaminergic neuron than GABAergic neuron, or more GABAergic neuron than dopaminergic neuron. The percentage of dopaminergic neurons and GABAergic neurons by total number of neurons in the spheroid may be preferably: at least 90% dopaminergic neurons, at least 10% GABAergic neurons; at least 80% dopaminergic neurons, at least 20% GABAergic neurons; at least 70% dopaminergic neurons, at least 30% GABAergic neurons; at least 60% dopaminergic neurons, at least 40% GABAergic neurons; at least 50% dopaminergic neurons, at least 50% GABAergic neurons; at least 40% dopaminergic neurons, at least 60% GABAergic neurons; at least 30% dopaminergic neurons, at least 70% GABAergic neurons; at least 20% dopaminergic neurons, at least 80% GABAergic neurons; or at least 10% dopaminergic neurons, at least 90% GABAergic neurons.

The composition comprises GABAergic neurons and glutamatergic neurons. Approximately equal amounts of the glutamatergic neuron and GABAergic neuron may be present in the composition. Alternatively, the composition may comprise more glutamatergic neuron than GABAergic neuron, or more GABAergic neuron than glutamatergic neuron. The percentage of glutamatergic neurons and GABAergic neurons by total number of neurons in the spheroid may be: at least 90% glutamatergic neurons, at least 10% GABAergic neurons; at least 80% glutamatergic neurons, at least 20% GABAergic neurons; at least 70% glutamatergic neurons, at least 30% GABAergic neurons; at least 60% glutamatergic neurons, at least 40% GABAergic neurons; at least 50% glutamatergic neurons, at least 50% GABAergic neurons; at least 40% glutamatergic neurons, at least 60% GABAergic neurons; at least 30% glutamatergic neurons, at least 70% GABAergic neurons; at least 20% glutamatergic neurons, at least 80% GABAergic neurons; or at least 10% glutamatergic neurons, at least 90% GABAergic neurons.

The composition comprises dopaminergic neurons and glutamatergic neurons. Approximately equal amounts of the glutamatergic neuron and dopaminergic neuron may be present in the composition. Alternatively, the composition may comprise more dopaminergic neuron than glutamatergic neuron, or more glutamatergic neuron than dopaminergic neuron. The percentage of glutamatergic neurons and dopaminergic neurons by total number of neurons in the spheroid may be: at least 90% dopaminergic neurons, at least 10% glutamatergic neurons; at least 80% dopaminergic neurons, at least 20% glutamatergic neurons; at least 70% dopaminergic neurons, at least 30% glutamatergic neurons; at least 60% dopaminergic neurons, at least 40% glutamatergic neurons; at least 50% dopaminergic neurons, at least 50% glutamatergic neurons; at least 40% dopaminergic neurons, at least 60% glutamatergic neurons; at least 30% dopaminergic neurons, at least 70% glutamatergic neurons; at least 20% dopaminergic neurons, at least 80% glutamatergic neurons; or at least 10% dopaminergic neurons, at least 90% glutamatergic neurons.

The composition comprises dopaminergic neurons, GABAergic neurons and glutamatergic neurons. Approximately equal amounts of the dopaminergic neuron, GABAergic, and glutamatergic neuron may be present in the composition. Alternatively, the composition may comprise more dopaminergic neuron than GABAergic and glutamatergic neuron, or more GABAergic neuron than dopaminergic or glutamatergic neuron, or more glutamatergic than dopaminergic and GABAergic neuron. The percentage of GABAergic, glutamatergic neurons and dopaminergic neurons by total number of neurons in the spheroid may be: at least 10% dopaminergic neurons, at least 10% GABAergic neurons, at least 80% glutamatergic neurons; at least 10% dopaminergic neurons, at least 20% GABAergic neurons, at least 70% glutamatergic neurons; at least 10% dopaminergic neurons, at least 30% GABAergic neurons, at least 60% glutamatergic neurons; at least 10% dopaminergic neurons, at least 40% GABAergic neurons, at least 50% glutamatergic neurons; at least 10% dopaminergic neurons, at least 50% GABAergic neurons, at least 40% glutamatergic neurons; at least 10% dopaminergic neurons, at least 60% GABAergic neurons, at least 30% glutamatergic neurons; at least 10% dopaminergic neurons, at least 70% GABAergic neurons, at least 20% glutamatergic neurons; at least 10% dopaminergic neurons, at least 80% GABAergic neurons, at least 10% glutamatergic neurons; at least 20% dopaminergic neurons, at least 10% GABAergic neurons, at least 70% glutamatergic neurons; at least 20% dopaminergic neurons, at least 20% GABAergic neurons, at least 60% glutamatergic neurons; at least 20% dopaminergic neurons, at least 30% GABAergic neurons, at least 50% glutamatergic neurons; at least 20% dopaminergic neurons, at least 40% GABAergic neurons, at least 40% glutamatergic neurons; at least 20% dopaminergic neurons, at least 50% GABAergic neurons, at least 30% glutamatergic neurons; at least 20% dopaminergic neurons, at least 60% GABAergic neurons, at least 20% glutamatergic neurons; at least 20% dopaminergic neurons, at least 70% GABAergic neurons, at least 10% glutamatergic neurons at least 30% dopaminergic neurons, at least 10% GABAergic neurons, at least 60% glutamatergic neurons; at least 30% dopaminergic neurons, at least 20% GABAergic neurons, at least 50% glutamatergic neurons; at least 30% dopaminergic neurons, at least 30% GABAergic neurons, at least 40% glutamatergic neurons; at least 30% dopaminergic neurons, at least 40% GABAergic neurons, at least 30% glutamatergic neurons; at least 30% dopaminergic neurons, at least 50% GABAergic neurons, at least 20% glutamatergic neurons; at least 30% dopaminergic neurons, at least 60% GABAergic neurons, at least 10% glutamatergic neurons; at least 40% dopaminergic neurons, at least 50% GABAergic neurons, at least 10% glutamatergic neurons; at least 40% dopaminergic neurons, at least 40% GABAergic neurons, at least 20% glutamatergic neurons; at least 40% dopaminergic neurons, at least 30% GABAergic neurons, at least 30% glutamatergic neurons; at least 40% dopaminergic neurons, at least 20% GABAergic neurons, at least 40% glutamatergic neurons; at least 40% dopaminergic neurons, at least 10% GABAergic neurons, at least 50% glutamatergic neurons; at least 50% dopaminergic neurons, at least 10% GABAergic neurons, at least 40% glutamatergic neurons; at least 50% dopaminergic neurons, at least 20% GABAergic neurons, at least 30% glutamatergic neurons; at least 50% dopaminergic neurons, at least 30% GABAergic neurons, 20% glutamatergic neurons; at least 50% dopaminergic neurons, at least 40% GABAergic neurons, at least 10% glutamatergic neurons; at least 60% dopaminergic neurons, at least 10% GABAergic neurons, at least 30% glutamatergic neurons; at least 60% dopaminergic neurons, at least 20% GABAergic neurons, at least 20% glutamatergic neurons; at least 60% dopaminergic neurons, at least 30% GABAergic neurons, at least 10% glutamatergic neurons; at least 70% dopaminergic neurons, at least 10% GABAergic neurons, at least 20% glutamatergic neurons; at least 70% dopaminergic neurons, at least 20% GABAergic neurons, at least 10% glutamatergic neurons; at least 80% dopaminergic neurons, at least 10% GABAergic neurons, at least 20% glutamatergic neurons; or at least 80% dopaminergic neurons, at least 20% GABAergic neurons, at least 10% glutamatergic neurons.

The spheroids described herein can comprise about 90% dopaminergic neurons and 10% GABAergic neurons by total number of cells in the spheroid.

The spheroids described herein can comprise about 80% dopaminergic neurons and 20% GABAergic neurons by total number of cells in the spheroid.

The spheroids described herein can comprise about 80% dopaminergic neurons, 10% glutaminergic neurons, and 10% GABAergic neurons by total number of cells in the spheroid.

The spheroids described herein can comprise about 60% dopaminergic neurons, 20% glutaminergic neurons, and 20% GABAergic neurons by total number of cells in the spheroid.

The spheroids described herein can comprise about 25% dopaminergic neurons, 25% glutaminergic neurons, and 50% GABAergic neurons by total number of cells in the spheroid. The spheroids described herein can comprise about 10% dopaminergic neurons, 10% glutaminergic neurons, and 80% GABAergic neurons by total number of cells in the spheroid.

The spheroids described herein can comprise about 80% dopaminergic neurons and 20% GABAergic neurons by total number of cells in the spheroid.

The spheroids described herein can comprise about 10% dopaminergic neurons and 90% GABAergic neurons by total number of cells in the spheroid.

The spheroids described herein can comprise about 10% dopaminergic neurons, 80% glutaminergic neurons, and 10% GABAergic neurons by total number of cells in the spheroid.

The spheroids described herein can comprise about 25% dopaminergic neurons, 50% glutaminergic neurons, and 25% GABAergic neurons by total number of cells in the spheroid.

The spheroids described herein can comprise about 50% dopaminergic neurons and 50% glutaminergic neurons by total number of cells in the spheroid.

The spheroids described herein can comprise about 33% dopaminergic neurons, 33% glutaminergic neurons, and 33% GABAergic neurons by total number of cells in the spheroid.

The spheroids described herein can comprise about 50% glutaminergic neurons and 50% GABAergic neurons by total number of cells in the spheroid.

The spheroids described herein can comprise about 90% dopaminergic neurons and 10% astrocytes by total number of cells in the spheroid.

The spheroids described herein can comprise about 90% glutaminergic neurons and 10% astrocytes by total number of cells in the spheroid.

The spheroids described herein can comprise about 90% GABAergic neurons and 10% astrocytes by total number of cells in the spheroid.

The spheroids described herein can comprise between about 1% and 100% astrocytes by total number of cells in the spheroid. The spheroids described herein can comprise between about 1%, 2%, 3%, 4%, 5%, 6%, 7%, 8%, 9%, 10%, 11%, 12%, 13%, 14%, 15%, 16%, 17%, 18%, 19%, or 20% neurons by total number of cells in the spheroid. The spheroids described herein can comprise between about 10% astrocytes by total number of cells in the spheroid.

The spheroids described herein can comprise between about 1% and 100% neurons by total number of cells in the spheroid. The spheroids described herein can comprise between about 80%, 81%, 82%, 83%, 84%, 85%, 86%, 87%, 88%, 89%, 90%, 91%, 92%, 93%, 94%, 95%, 96%, 97%, 98%, 99%, or 100% neurons by total number of cells in the spheroid. The spheroids described herein can comprise between about 90% neurons by total number of cells in the spheroid.

The spheroids described herein can comprise motor neurons. The spheroid may comprise motor neurons in an amount between about 1% and 50% by total amount of cells.

The spheroids described herein can comprise microglia. The spheroid may comprise microglia in an amount between about 1% and 50% by total amount of cells.

The spheroids described herein can be assembled using the amounts of neurons, glia, pericytes, endothelial cells, and combinations thereof, described herein, optionally using differentiated neurons.

The spheroids may form brain-specific-region like architecture that models brain regions. Example brain regions include prefrontal cortex (PFC), nucleus accumbens, amygdala, hippocampus, and ventral tegmental area (VTA). These spheroids are referred to as prefrontal cortex (PFC)-like, nucleus accumbens-like, amygdala-like, hippocampus-like, and ventral tegmental area (VTA)-like. In some embodiments, the brain specific-region spheroids comprise combination of neuron and glia cells as shown in FIG. 3. In some embodiments, the PFA-like spheroids may comprise combination of glutamatergic neurons, GABAergic neurons, and Astrocytes. The VTA-like spheroids may comprise combination of dopaminergic neurons, GABAergic neurons, Glutamatergic neurons, and astrocytes. The nucleus accumbens-like spheroids may comprise combinations of GABAergic neurons and astrocytes. The hippocampus-like spheroids comprise combinations glutamatergic neurons, GABAergic neurons, and astrocytes.

VTA-like spheroids may be characterized by the presence of dopamine-producing cells, which occurs only here and in the substantia nigra (SN) in vivo. Previous studies show increased GABAergic populations in VTA that are not present in SN (Nair-Roberts, R. G., Chatelain-Badie, S. D., Benson, E., et al. “Stereological estimates of dopaminergic, GABAergic and glutamatergic neurons in the ventral tegmental area, substantia nigra and retrorubral field in the rat.” Neuroscience. 2008, 152(4): 1024-31; Root, D. H., Wang, H. L., Liu, B., et al. “Glutamate neurons are intermixed with midbrain dopamine neurons in nonhuman primates and humans.” Sci Rep. 2016, 6: 30615). Furthermore, histological studies have shown the VTA to be comprised of 55-65% dopaminergic neurons, 30-35% GABA neurons and 2-5% glutamatergic neurons (see review: Pignatelli, M. and A. Bonci. “Role of Dopamine Neurons in Reward and Aversion: A Synaptic Plasticity Perspective.” Neuron. 2015, 86(5): 1145-57). To model this brain region in spheroids, the inventors produced spheroids consisting of these three neuronal subtypes in controlled ratios intended to reflect what has been shown in vivo. To further validate these, the inventors modeled aspects of addiction, specifically opioid use disorder (OUD), given that the VTA is at the forefront of reward circuitry in the brain, with studies showing that drug reward increases dopamine release (see review: Scofield, M. D., Heinsbroek, J. A., Gipson, C. D., et al. “The Nucleus Accumbens: Mechanisms of Addiction across Drug Classes Reflect the Importance of Glutamate Homeostasis.” Pharmacol Rev. 2016, 68(3): 816-71). To model aspects of OUD, there were two groups of spheroids: 1. spheroids that had been chronically treated with DAMGO (mu opioid receptor agonist) to model drug dependence or 2. Spheroids that had been chronically treated and subjected to 3-days withdrawal to model forced abstinence from a drug after a period of dependence. To model relapse, spheroids of both groups were then challenged with one final dose of DAMGO again and calcium activity was recorded using a FLIPR 1- and 30-min after exposure. In our model, similar to what has been shown in vivo in among humans and animals, baseline calcium activity within these two groups was hyperactive compared to controls (Koo, J. W., Mazei-Robison, M. S., Chaudhury, D., et al. “BDNF is a negative modulator of morphine action.” Science. 2012, 338(6103): 124-8; Meye, F. J., van Zessen, R., Smidt, M. P., et al. Morphine withdrawal enhances constitutive mu-opioid receptor activity in the ventral tegmental area. J Neurosci. 2012, 32(46):16120-8; Yang, Z., Xie, J., Shao, Y., et al. Dynamic neural responses to cue-reactivity paradigms in heroin-dependent users: an fMRI study. Human Brain Mapp. 2009, 30(3):766-75; Zijlstra, F., Veltman, D. J., Booji, J., et al. Neurobiological substrates of cue-elicited craving and anhedonia in recently abstinent opioid-dependent males. Drug Alcohol Depend. 2009, 99(1-3): 183-92) Furthermore, naloxone, a mu opioid receptor antagonist used to reverse opioid overdose in humans, was exposed to spheroids 40-min after they were treated with DAMGO, and calcium activity was restored to control levels.

PFC-like spheroids may be characterized by the ratio of neuronal subtypes (glutamatergic and GABAergic) that make up this region in vivo. Previous studies show that 70-75% of the prefrontal cortex (PFC) consists of glutamatergic neurons and 25-30% (see review: Ghosal, S., Hare, B., Duman, S. “Prefrontal Cortex GABAergic Deficits and Circuit Dysfunction in the Pathophysiology and Treatment of Chronic Stress and Depression.” Curr Opin Behav Sci. 2017, 14: 1-8). To model this brain region in spheroids, the inventors produced spheroids consisting of these neuronal subtypes in controlled ratios intended to reflect what has been shown in vivo. To further validate these, the inventors modeled aspects of addiction, specifically opioid use disorder (OUD), given that the prefrontal cortex regulates impulsivity and decision-making and is therefore involved in relapse behaviors (see review: Scofield, M. D., Heinsbroek, J. A., Gipson, C. D., et al. “The Nucleus Accumbens: Mechanisms of Addiction across Drug Classes Reflect the Importance of Glutamate Homeostasis.” Pharmacol Rev. 2016, 68(3): 816-71. To model aspects of OUD, there were two groups of spheroids: 1. spheroids that had been chronically treated with DAMGO (mu opioid receptor agonist) to model drug dependence or 2. Spheroids that had been chronically treated and subjected to 3-days withdrawal to model forced abstinence from a drug after a period of DAMGO withdrawal. To model relapse, spheroids of both groups were then challenged with one final dose of DAMGO again and calcium activity was recorded using a FLIPR 1- and 30-min after exposure. In our model, similar to what has been shown in vivo in among humans and animals, baseline calcium activity within these two groups was hypoactive compared to controls (Chang, J. Y., Zhang, L., Janack, P. H., et al. Neuronal responses in prefrontal cortex and nucleus accumbens during heroin self-administration in freely moving rats. Brain Res. 1997, 754(1-2):12-20; Nui, H., Zhang, G., Li, H., et al. Multi-system state shifts and cognitive deficits induced by chronic morphine during abstinence. Neurosci Lett. 2017, 640:144-15; Robinson, T. E. and B. Kolb. Morphine alters the structure of neurons in the nucleus accumbens and neocortex or rats. Synapse 1999, 33(2): 160-2.

Furthermore, naloxone, a mu opioid receptor antagonist used to reverse opioid overdose in humans, was exposed to spheroids 40-min after they were treated with DAMGO, and calcium activity was restored to control levels. This is in line with human data showing that treatment with buprenorphine, a mu opioid receptor partial agonist, reduces signal activation within the PFC of people suffering from OUD to the levels of healthy controls (Langleben et al. Am J Psychiatry. 2008, 165(3):390-4; Mei et al. Neuroscience. 2010, 170(3): 808-15).

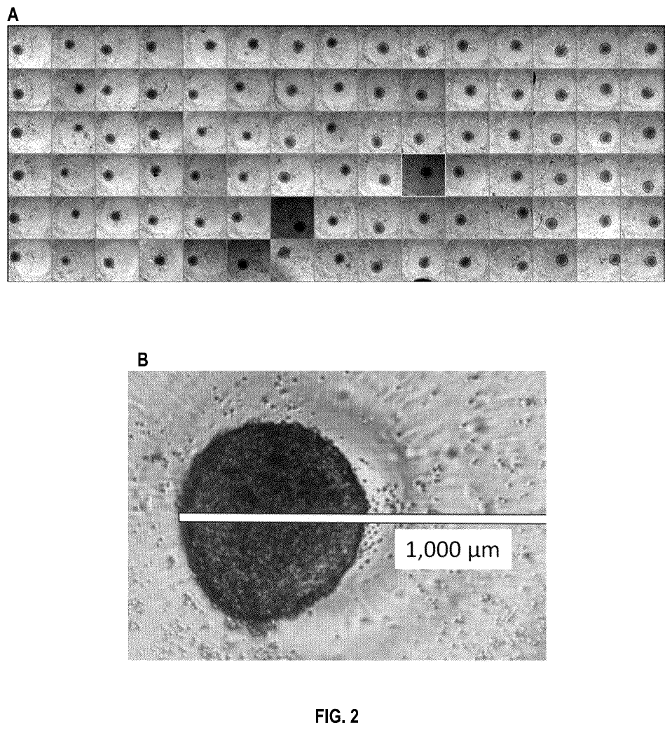

The spheroids have a diameter of about 1 μm to 1,000 μm. For example, spheroids may a diameter of about 20-100 μm, 30-100 μm, 40-100 μm, 50-100 μm, 60-100 μm, 70-100 μm, 80-100 μm, 20-80 μm, 30-80 μm, 40-80 μm, 50-80 μm, 20-60 μm, 30-60 μm, or 40-60 μm. The spheroids have a diameter of about 20 μm, 30 μm, 40 μm, 50 μm, 60 μm, 70 μm, 80 μm, 90 μm, or 100 μm. The spheroids have a diameter of about 100 μm, 200 μm, 300 μm, 400 μm, 500 μm, 600 μm, 700 μm, 800 μm, 900 μm, or 1,000 μm. The spheroids may have a diameter between about 100 μm and 500 μm, 250 μm and 750 μm, 300 μm and 900 μm, or 400 μm and 600 μm. FIG. 2 (A & B).

### Vessel

The spheroid may be plated in a suspension or adherent culture. The spheroids may grow in suspension. The spheroids may be plated in a vessel including but not limited to a multi-well plate, flask, dish, tube, and tank. A preferred vessel is a multi-well plate, for example, a 4-well cell culture plate, a 6-well cell culture plate, a 8-well cell culture plate, a 12-well cell culture plate, a 24-well cell culture plate, a 48-well cell culture plate, a 96-well cell culture plate, a 384-well cell culture plate, or 1536-well cell culture plate. The vessel may comprise an ultra-low attachment surface (ULA).

The solid surface may have a length, width, and/or diameter of 2 mm to 10 mm, and/or a height of 2 mm to 10 mm. The solid surface may have a length, width, and/or diameter of 2 mm to 20 mm. The solid surface may have a length, width, and/or diameter of 2 mm to 25 mm. The solid surface may have a length, width, and/or diameter of 1 mm to 50 mm. The solid surface may have a length, width, and/or diameter of 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, 40 mm, 41 mm, 42 mm, 43 mm, 44 mm, 45 mm, 46 mm, 47 mm, 48 mm, 49 mm, or 50 mm. The solid surface may have a length, width, and/or diameter of at least 2 mm. The solid surface may have a length, width, and/or diameter of less than or equal to 50 mm.

The solid surface may have height of 2 mm to 25 mm. The solid surface may have a height of 1 mm to 50 mm. The solid surface may have a height of 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, 40 mm, 41 mm, 42 mm, 43 mm, 44 mm, 45 mm, 46 mm, 47 mm, 48 mm, 49 mm, or 50 mm. The solid surface may have a height of at least 2 mm. The solid surface may have a height of less than or equal to 50 mm.

### Quality Control Processes

Screening methods and quality control processes may be employed to obtain brain specific-region spheroids. Optical assays such as Fluorescent Imaging Plate Reader (FLIPR) may be used for evaluating agonists and antagonists via calcium signaling. Calcium uptake fluorescence oscillations, compound addition assays, neurotransmitter release assays, and combinations thereof may be used to characterize the spheroids.

### Use of Brain Specific-Region Spheroids

The formed brain region-specific spheroids can be used to test the effects of agents on neurons and neuronal function. The brain region-specific spheroids may be used in the testing and discovery of new drugs and treatments.

The brain region-specific spheroids are used to test addictive behaviors to opioids. μ-Opioid receptors (MORs) in the ventral tegmental area (VTA) are pivotally involved in addictive behavior. To test activity in VTA-like spheroids, spheroids are administered DAMGO, a synthetic opioid peptide with high μ-opioid receptor specificity. DAMGO has been used in experimental settings for the possibility of alleviating or reducing opiate tolerance for patients under the treatment of an opioid. An opioid use disorder (OUD) is modeled by showing selective disruption in “VTA-like” and “PFC-like” spheroid calcium activity after chronic exposure to opioids that are ameliorated by naloxone, which is used to reverse opioid overdose in humans.

### Disease Models

Spheroids may be designed using the methods described herein for creating diseases specific spheroid models. For example, disease specific spheroid models may be designed for opioid use disorder (OUD), Parkinson's disease, Alzheimer's disease, Huntington's Disease (HD), Autism spectrum disorder, Rett Syndrome, Dravet Syndrome, dementia, epilepsy, Amyotrophic lateral sclerosis, or Down Syndrome. Disease specific spheroid models designed for opioid use disorder (OUD), Parkinson's disease, Alzheimer's disease, Huntington's Disease (HD), Autism spectrum disorder, Rett Syndrome, Dravet Syndrome, dementia, epilepsy, Amyotrophic lateral sclerosis, Down Syndrome can comprise neurons from iPSC lines from affected patients or CRISPR engineered, from any modeled brain region.

In reference to FIG. 3, this drawing depicts a mapping of exemplary region-specific spheroids as described herein and their corresponding area in the mammalian brain (a human brain is shown for illustrative purposes). The spheroids described herein can be designed and produced to model relevant brain regions by combining differentiated neurons, including iPSC-derived differentiated neurons, and used to study scientifically- and clinically-relevant topics including (but not limited to) opioid use disorder (OUD), Parkinson's disease, Alzheimer's disease, Huntington's Disease (HD), Autism spectrum disorder, Rett Syndrome, Dravet Syndrome, dementia, epilepsy, Amyotrophic lateral sclerosis, or Down Syndrome.

In reference to FIG. 7, this drawing depicts a flow chart with an exemplary method for characterization and use of the spheroids described herein. The last three steps may be modified to fit a specific application and are extendable to other disorders/diseases, e.g., neurodegenerative disorders including but not limited to Alzheimer's Disease, Parkinson's disease, and Huntington's Disease.

### Alzheimer's Disease

To model Alzheimer's Disease (AD), GABA neurons that were genetically engineered to express the apolipoprotein e4 (APOE4) allele, a genotype associated with AD, were incorporated into spheroids on the day they were generated. In an embodiment, neurons and glial from Alzheimer's Disease patients may be incorporated into spheroids on the day the spheroids were generated. Spheroids modeling the prefrontal cortex (PFC-like spheroids) were used for these experiments. They were 90% neuron 10% astrocyte and neuronal composition consisted of 70% glutamatergic and 30% GABAergic neurons. For example, spheroids for testing Alzheimer's Disease can be modeled after the hippocampus and comprise about 80% glutamatergic neurons and 20% GABAergic neurons by total number of neurons.

The AD Spheroids described herein may be used in methods for studying Alzheimer's Disease.

The AD Spheroids described herein may be used in methods for screening compounds for activity in treating Alzheimer's Disease. For example, the AD spheroids may be cultured in a culture vessel, test compounds added at varying concentrations, and the activity of the AD spheroids examined, including neural activity, cell death, and/or development of amyloid plaques. A test compound that improves neural activity, reduces cell death, and/or reduces the development of amyloid plaques, or increases clearance of the amyloid plaques, may be identified as a potential therapeutic for AD. The neural activity can be electrophysiological properties, calcium activity profile, neurotransmitter release, or a combination thereof.

### Parkinson's Disease

To model Parkinson's Disease (PD), the inventors incorporated dopaminergic neurons expressing A53T mutant alpha-synuclein into spheroids given that it is a common risk factor for non-familial Parkinson's Disease (PD). In an embodiment, neurons and glial from Parkinson's Disease patients may be incorporated into spheroids on the day the spheroids were generated. Spheroids modeling the ventral tegmental area (VTA-like spheroids) were used for these experiments. They were 90% neuron 10% astrocyte and neuronal composition consisted of 65% dopaminergic 5% glutamatergic and 30% GABAergic neurons.

The PD Spheroids described herein may be used in methods for studying Parkinson's Disease.

The PD Spheroids described herein may be used in methods for screening compounds for activity in treating Parkinson's Disease. For example, the PD spheroids may be cultured in a culture vessel, test compounds added at varying concentrations, and the activity of the PD spheroids examined, including neural activity, cell death, and/or development of Lewy Bodies (LB). A test compound that improves neural activity, cell death, and/or reduces the development of Lewy Bodies, or increases clearance of the Lewy Bodies, may be identified as a potential therapeutic for PD. The neural activity can be electrophysiological properties, calcium activity profile, neurotransmitter release, or a combination thereof.

The spheroids, for example AD spheroids or PD spheroids, may be plated in a suspension or adherent culture for study and/or screening methods. The spheroids may grow in suspension. The spheroids may be plated in a vessel including but not limited to a multi-well plate, flask, dish, tube, and tank. A preferred vessel is a multi-well plate, for example, a 4-well cell culture plate, a 6-well cell culture plate, a 8-well cell culture plate, a 12-well cell culture plate, a 24-well cell culture plate, a 48-well cell culture plate, a 96-well cell culture plate, a 384-well cell culture plate, or 1536-well cell culture plate. The vessel may comprise an ultra-low attachment surface (ULA). The spheroids, for example AD spheroids or PD spheroids, may be plated in a suspension or adherent culture for high-throughput screening methods. The spheroids described herein may be used in high-throughput screening systems and methods.

The spheroid described herein may be transfected cells transfected using a viral construct. The viral construct can be an adeno-associated virus, optionally AAV9. The cells of the spheroids described herein can be transfected with a transgene. The transgene can be A53T mutant alpha-synuclein (PD models), or APOE4 (AD models).

### Classification Systems

The invention relates to, among other things, characterizing compounds based on their activity in spheroid cultures, preferably the affect compounds have on activity of spheroid cultures. The data collected from screening compounds using the spheroids described herein may be analyzed using machine learning to classify the compounds. The classification systems used herein may include computer executable software, firmware, hardware, or combinations thereof. For example, the classification systems may include reference to a processor and supporting data storage. Further, the classification systems may be implemented across multiple devices or other components local or remote to one another. The classification systems may be implemented in a centralized system, or as a distributed system for additional scalability. Moreover, any reference to software may include non-transitory computer readable media that when executed on a computer, causes the computer to perform a series of steps.

The classification systems described herein may include data storage such as network accessible storage, local storage, remote storage, or a combination thereof. Data storage may utilize a redundant array of inexpensive disks (“RAID”), tape, disk, a storage area network (“SAN”), an internet small computer systems interface (“iSCSI”) SAN, a Fibre Channel SAN, a common Internet File System (“CIFS”), network attached storage (“NAS”), a network file system (“NFS”), or other computer accessible storage. The data storage may be a database, such as an Oracle database, a Microsoft SQL Server database, a DB2 database, a MySQL database, a Sybase database, an object oriented database, a hierarchical database, Cloud-based database, public database, or other database. Data storage may utilize flat file structures for storage of data. Exemplary embodiments used two Tesla K80 NVIDIA GPUs, each with 4992 CUDA cores and large amounts of GB of memory (e.g., over 10 GB) to train the deep learning algorithms.

In the first step, a classifier is used to describe a pre-determined set of data. This is the “learning step” and is carried out on “training” data.

The training database is a computer-implemented store of data reflecting the activity of a compound with a classification with respect to the activity of the compound. The data can comprise electrophysiological data, gene expression, calcium activity, neurotransmitter release and/or re-uptake, or a combination thereof. The format of the stored data may be as a flat file, database, table, or any other retrievable data storage format known in the art. The test data may be stored as a plurality of vectors, each vector corresponding to an individual compound, each vector including a plurality of compound data measures for a plurality of experimental compounds data together with a classification with respect to activity characterization of the compound. Typically, each vector contains an entry for each compound data measure in the plurality of compound data measures. The entry can further comprise electrophysiological data, gene expression, calcium activity, neurotransmitter release and/or re-uptake, cell death (apoptosis, necrosis), or a combination thereof. The training database may be linked to a network, such as the internet, such that its contents may be retrieved remotely by authorized entities (e.g., human users or computer programs). Alternately, the training database may be located in a network-isolated computer. Further, the training database may be Cloud-based, including proprietary and public databases containing compound data (e.g., experimental, predicted, and combinations thereof).

In the second step, which is optional, the classifier is applied in a “validation” database and various measures of accuracy, including sensitivity and specificity, are observed. In an exemplary embodiment, only a portion of the training database is used for the learning step, and the remaining portion of the training database is used as the validation database. In the third step, compound activity data measures from a subject are submitted to the classification system, which outputs a calculated classification (e.g., characterization of a compound as antagonist, characterization of the compound as a potential Alzheimer's therapeutic) for the subject.

There are many possible classifiers that could be used on the data. Machine and deep learning classifiers include but are not limited to AdaBoost, Artificial Neural Network (ANN) learning algorithm, Bayesian belief networks, Bayesian classifiers, Bayesian neural networks, Boosted trees, case-based reasoning, classification trees, Convolutional Neural Networks, decisions trees, Deep Learning, elastic nets, Fully Convolutional Networks (FCN), genetic algorithms, gradient boosting trees, k-nearest neighbor classifiers, LASSO, Linear Classifiers, naive Bayes classifiers, neural nets, penalized logistic regression, Random Forests, ridge regression, support vector machines, or an ensemble thereof, may be used to classify the data. See e.g., Han & Kamber (2006) Chapter 6, Data Mining, Concepts and Techniques, 2nd Ed. Elsevier: Amsterdam. As described herein, any classifier or combination of classifiers (e.g., ensemble) may be used in a classification system. As discussed herein, the data may be used to train a classifier.

### Classification Trees

A classification tree is an easily interpretable classifier with built in feature selection. A classification tree recursively splits the data space in such a way so as to maximize the proportion of observations from one class in each subspace.

The process of recursively splitting the data space creates a binary tree with a condition that is tested at each vertex. A new observation is classified by following the branches of the tree until a leaf is reached. At each leaf, a probability is assigned to the observation that it belongs to a given class. The class with the highest probability is the one to which the new observation is classified.

Classification trees are essentially a decision tree whose attributes are framed in the language of statistics. They are highly flexible but very noisy (the variance of the error is large compared to other methods).

Tools for implementing classification tree are available, by way of non-limiting example, for the statistical software computing language and environment, R. For example, the R package “tree,” version 1.0-28, includes tools for creating, processing and utilizing classification trees. Examples of Classification Trees include but are not limited to Random Forest. See also Kamiski et al. (2017) “A framework for sensitivity analysis of decision trees.” Central European Journal of Operations Research. 26(1): 135-159; Karimi & Hamilton (2011) “Generation and Interpretation of Temporal Decision Rules”, International Journal of Computer Information Systems and Industrial Management Applications, Volume 3, the content of which is incorporated by reference in its entirety.

### Screening Methods

The spheroids described herein may be used in methods of screening compounds to, for example, identify potentially therapeutic compounds. For example, disease specific spheroid models may be designed for opioid use disorder (OUD), Parkinson's disease, Alzheimer's disease, Huntington's Disease (HD), Autism spectrum disorder, Rett Syndrome, Dravet Syndrome, dementia, epilepsy, Amyotrophic lateral sclerosis, Down Syndrome may be used in methods of screening compounds to identify potentially therapeutic compounds.

A method of screening compounds can comprise (a) culturing a spheroid described herein, optionally a disease specific spheroid models may be designed for opioid use disorder (OUD), Parkinson's disease, Alzheimer's disease, Huntington's Disease (HD), Autism spectrum disorder, Rett Syndrome, Dravet Syndrome, dementia, epilepsy, Amyotrophic lateral sclerosis, Down Syndrome; (b) exposing the spheroid to a test compound; and (c) measuring activity and collecting activity data.

A method of screening compounds can comprise (a) culturing a spheroid described herein, optionally a disease specific spheroid models may be designed for opioid use disorder (OUD), Parkinson's disease, Alzheimer's disease, Huntington's Disease (HD), Autism spectrum disorder, Rett Syndrome, Dravet Syndrome, dementia, epilepsy, Amyotrophic lateral sclerosis, Down Syndrome; (b) exposing the spheroid to a test compound; (c) measuring activity and collecting activity data; and (d) classifying the activity data using machine learning to produce a classification on the activity affected by the compound.

A method of identifying spheroids that accurately model a disease state can comprise (a) culturing a disease specific spheroid models may be designed for opioid use disorder (OUD), Parkinson's disease, Alzheimer's disease, Huntington's Disease (HD), Autism spectrum disorder, Rett Syndrome, Dravet Syndrome, dementia, epilepsy, Amyotrophic lateral sclerosis, Down Syndrome, described herein; (b) testing physiological, cellular, and genetic properties of the spheroid; (c) measuring activity and collecting activity data; and (d) classifying the activity data using machine learning to produce a classification on how accurately the spheroid models the disease.

A method of screening compounds can comprise (a) culturing an assembloid comprising at least two spheroids described herein in a matrix; (b) exposing the spheroid to a test compound; and (c) measuring activity and collecting activity data. The matrix can be collagen, laminin, fibronectin, hydrogels, and combinations thereof. The two spheroids can be a PFC spheroid and a VTA spheroid.

A method of screening compounds can comprise (a) culturing an assembloid comprising at least two spheroids described herein in a matrix; (b) exposing the spheroid to a test compound; (c) measuring activity and collecting activity data; and (d) classifying the activity data using machine learning to produce a classification on the activity affected by the compound. The matrix can be collagen, laminin, fibronectin, hydrogels, and combinations thereof. The two spheroids can be a PFC spheroid and a VTA spheroid. A random forest model machine learning classifier model was used to quantify predictability of labeling disease phenotype and showed high accuracy for both the AD and PD models (>94%).

The spheroid used in the screening methods described herein can comprise about 25% GABAergic neurons, 65% glutamatergic neurons, and 10% astrocytes by total percentage of cell number per spheroid and exhibits the properties of cells from the prefrontal cortex (PFC).

The spheroid used in the screening methods described herein can comprise about 60% dopaminergic neurons, 27.5% GABAergic neurons, 2.5% glutamatergic neurons, and 10% astrocytes by total percentage of cell number per spheroid and exhibits the properties of cells from the ventral tegmental area (VTA).

The screening methods described herein can use a classification system selected from the group consisting of AdaBoost, Artificial Neural Network (ANN) learning algorithm, Bayesian belief networks, Bayesian classifiers, Bayesian neural networks, Boosted trees, case-based reasoning, classification trees, Convolutional Neural Networks, decisions trees, Deep Learning, elastic nets, Fully Convolutional Networks (FCN), genetic algorithms, gradient boosting trees, k-nearest neighbor classifiers, LASSO, Linear Classifiers, Naive Bayes, neural nets, penalized logistic regression, Random Forests, ridge regression, support vector machines, or an ensemble thereof. The classification system can be an ensemble of classification systems. The prediction performance score can be greater than about 0.95. The prediction performance score can be from about 0.92 to about 0.98 or at about 0.92, 0.93, 0.94, 0.95, 0.96, 0.97, or 0.98.

The activity measured can be cell death, calcium activity, neurotransmitter release, neurotransmitter uptake, or a combination thereof.

The methods described herein can be a high-throughput screening method.

Throughout this application, various publications are referenced. The disclosures of all of these publications and those references cited within those publications in their entireties are hereby incorporated by reference into this application in their entirety in order to more fully describe the state of the art to which this invention pertains.

### EXAMPLES

### Example 1

### Spheroid Creation and Characterization

### Materials and Methods

### Cells

Frozen vials of human induced pluripotent stem cell (iPSC) derived, matured neurons were purchased from Cellular Dynamics (CDI)/Fujifilm—(Astrocytes, DopaNeurons, GABAneurons, GlutaNeurons, Microglia).

### Plates

96-well and 384-well black/clear bottom ultra-low attachment (ULA), round bottom plates were purchased from Corning Life Sciences (Glendale, Arizona).

### Media

Spheroid formation medias: CDI accompanying neural media was used during thawing of neurons/astrocytes as well as the first two days of neuronal spheroid formation (customized ratios of neural basal medium, neural basal supplement A, neural basal supplement B, nervous system (NS) supplement; ratios described below). Dopaminergic and glutamatergic neuron specific media, called Neural Basal Media B here, was comprised of 100 ml neural basal medium, 2 ml neural basal supplement B, and 1 ml nervous system (NS) supplement. GABAergic neuron specific media, referred to as Neural Basal Media A here, was comprised of 100 ml neural basal medium, and 2 ml neural basal supplement A. Astrocytes were diluted directly in neural basal medium. Spheroid maturation and maintenance media: spheroids were matured and maintained in a Brain Phys Neuronal medium with added supplements. Components were purchased as follows and used at final concentration indicated in table. All media was used within one week of formulation; ascorbic acid and cAMP were added day of media changes.

TABLE 1
Brain Phys Neuronal Medium Composition

| Brain Phys Neuronal | |
|---|---|
| Medium | StemCell Technologies |
| 1X N2 (100X) | Thermo, 17502-048 or |
| | 175020-01 |
| 1X B27 (50X) | Thermo, 17504 |
| 20 ng/mL BDNF | Stem Cell Tech, 78005 |
| 20 ng/mL GDNF | Stem Cell Tech, 78058 |
| 1 μg/mL Laminin | Invitrogen, 23017-015 |
| | (1 mg/mL) |
| 20 nM ascorbic acid | Tocris, 4055 (50 mg) |
| 1 mM cAMP | Tocris, 1141 (50 mg) |

### Formation of Spheroids

On day of spheroid formation, necessary neuronal cell vials were removed from −150° C. storage and kept on dry ice until thawed. Each vial of iPSC-derived neurons or astrocytes were thawed in a 37° C. water bath until just thawed, not to exceed times listed: GABANeurons (3 minutes), Dopa Neurons (3 minutes), GlutaNeurons (2 minutes), Astrocytes (3 minutes). After thawing, iDopaNeurons and GlutaNeurons were gently resuspended with a pre-rinsed, wide bore 1 ml pipet in 1 ml room temperature Neural Basal Media B and placed into a sterile 50 ml conical tube. An additional 1 ml room temperature Neural Basal Media B was used to rinse the cryovial of residual cells and added slowly in a dropwise fashion to the 50 ml conical containing DopaNeurons or GlutaNeurons, while gently swirling to minimize osmotic shock. An additional 8 ml of room temperature Neural Basal Media B was slowly added in dropwise fashion (˜2-3 drops/sec) to the 50 ml conical containing the cells, while gently swirling the conical to ensure even media mixing. Both GABA Neurons and astrocytes were handled the same way, but with Neural Basal Media A for GABA Neurons and Neural Basal Medium used for astrocytes. After resuspension, cells were counted using trypan blue and an automated cell counter. Only batches with over 50% viable cells were used for spheroid formation. Cells were then placed into individual 50 ml conical tubes and centrifuged at 300×g-400×g for 5 minutes. After centrifugation, the supernatant was carefully removed using a 5 ml pipettor, with 1 ml left in the centrifuge tube. Cells were gently resuspended in the residual 1 ml, counted, and further diluted to achieve desired concentrations for spheroid formation, at a final volume of 10,000 cells/50 μl media per 384-well spheroid, or 30,000-70,000 cells/200 μl cells per 96-well spheroid. A preferred number of cells for 384-well plates is about 10,000 total cells/50 μl/well, the range may be from 5,000 cells to 30,000 cells).

For formation of 16 spheroids, the following ratios of cells were tested and diluted in formation media as listed.

TABLE 2
Neuron Ratio; Astrocyte Ratio; Formation Media

| | Astrocyte Ratio | |
|---|---|---|
| Neuron Ratios | (% of total | |
| (% of total | cell number per | |
| neurons) | spheroid) | Formation Media |
| 100% Dopa Neurons | 10% astrocytes | Neural Basal Media B |
| 100% GABA Neurons | | Neural Basal Media A |
| 100% Gluta Neurons | | Neural Basal Media B |
| 90% Dopa Neurons, | | 9 part:1 part Neural Basal |
| 10% GABA Neurons | | Media B:Neural Basal |
| | | Media A |
| 80% Dopa Neurons, | | 8 part:2 part Neural Basal |
| 20% GABA Neurons | | Media B:Neural Basal |
| | | Media A |
| 90% GABA Neurons, | | 9 part:1 part Neural Basal |
| 10% Dopa Neurons | | Media A:Neural Basal |
| | | Media B |
| 80% GABA Neurons, | | 8 part:2 part Neural Basal |
| 20% Dopa Neurons | | Media A:Neural Basal |
| | | Media B |
| 33% Dopa Neurons, | | 7 part:3 part Neural Basal |
| 33% GABA Neurons, | | Media A:Neural Basal |
| 33% Gluta Neurons | | Media B |
| 50% GABA Neurons, | | 1 part:1 part Neural Basal |
| 50% Gluta Neurons | | Media A:Neural Basal |
| | | Media B |
| 50% Dopa Neurons, | | Neural Basal Media B |
| 50% Gluta Neurons | | |
| 80% Dopa Neurons, | | 9 part:1 part Neural Basal |
| 10% GABA Neurons, | | Media B:Neural Basal |
| 10% Gluta Neurons | | Media A |
| 60% Dopa Neurons, | | 8 part:2 part Neural Basal |
| 20% GABA Neurons, | | Media B:Neural Basal |
| 20% Gluta Neurons | | Media A |
| 25% Dopa Neurons, | | 1 part:1 part Neural Basal |
| 50% GABA Neurons, | | Media A:Neural Basal |
| 25% Gluta Neurons | | Media B |
| 10% Dopa Neurons, | | 8 part:2 part Neural Basal |
| 80% GABA Neurons, | | Media A:Neural Basal |
| 10% Gluta Neurons | | Media B |
| 25% Dopa Neurons, | | 1 part:1 part Neural Basal |
| 25% GABA Neurons, | | Media A:Neural Basal |
| 50% Gluta Neurons | | Media B |
| 10% Dopa Neurons, | | 9 part:1 part Neural Basal |
| 10% GABA Neurons, | | Media B:Neural Basal |
| 80% Gluta Neurons | | Media A |

For ventral tegmental area (VTA) like spheroids and prefrontal cortex (PEG) like spheroids, the ratios and media listed in Table 3 were used.

TABLE 3
Neuron Ratio; Astrocyte Ratio; Formation Media

| | Astrocyte Ratio | |
|---|---|---|
| Neuron Ratios | (% of total | |
| (% of total | cell number per | |
| neurons) | spheroid) | Formation Media |
| VTA-like spheroids | 10% astrocytes | 70% Neural Basal Media B |
| 65% Dopa Neurons | | 30% Neural Basal Media A |
| 5% Gluta Neurons | | |
| 30% GABA Neurons | | |
| PFC-like spheroids | | 70% Neural Basal Media B |
| 70% Gluta Neurons | | 30% Neural Basal Media A |
| 30% GABA Neurons | | |

After cells were mixed with indicated cell ratios in formation media in final ratios indicated above, cells were placed into individual reservoirs and gently swirled to mix. 10,000 cells/50 l were added to each individual 384-well ULA, round bottom plates using a multichannel pipet. 30,000 to 70,000 cells/200 l were added to 96-well ULA, round bottom plates. Cells were rested for 10 minutes prior to centrifugation in plates for 10 minutes at 1000 rpm (˜180×g). After centrifugation, plates were then placed at 37° C., 5% CO₂ incubator for two days without disturbance.

### Maintenance of Spheroids

Two days after initial formation, 5 l of media was removed from 384-well plates. 45 l of freshly made complete spheroid maturation media (Brain Phys Neuronal Media supplemented with 1×N2, 1× B27, 20 ng/ml BDNF, 20 ng/ml GDNF, 1 μg/ml laminin, 20 nM ascorbic acid, and 1 mM cAMP) was added to wells, for a final volume of 90 μl per well. For 96-well plates, 100 μl of media was removed from the 200 μl total and replaced with 100 l complete spheroid maturation media. Half-media changes were then completed every other day for a total of 3 weeks.

### Calcium Activity Profiling

To examine neuronal activity changes as a response to compound treatment, calcium activity was assessed using a Fluorescent Imaging Plate Reader (FLIPR) Penta (Molecular Devices). FLIPR Calcium 6 Assay kits were purchased from Molecular Devices (PN: R8190). On day of activity profiling, 96-well or 384-well ULA round bottom (black sides, clear bottom) plates containing spheroids were removed from incubator. Fresh FLIPR Calcium 6 dye was prepared by diluting each vial in 10 ml of freshly prepared, phenol-free complete spheroid maturation media and vortexed for 2 minutes. Half of the media was removed from each well (45 μl from 384-well plate wells, 100 ul from 96-well plate wells) and replaced with equal amounts freshly prepared FLIPR Calcium 6 dye, followed by a 2-hour incubation in the dark in a 37 C, 5% CO₂ incubator. After 2-hours, plates were removed and kept at room temperature for 10 minutes, then imaged and calcium activity read using the Molecular Devices FLIPR Penta High-Throughput Cellular Screening System. The FLIPR incubator stage was pre-warmed to 37° C. approximately 30 minutes prior to imaging. Standard filter sets were used for Cal6 imaging (excitation 470-495 and emission 515-575 nm). FLIPR recordings were obtained with the camera in normal mode, and settings included: gain was set to 2.5, exposure time to 0.03 seconds, and excitation intensity to 50%. Fluorescent image reads were taken every 0.6 seconds for a total of 1000 reads over a total run time of 10 minutes. Four recordings total were captured: baseline activity, 1-min after compound treatment, 30- and 70-min after compound treatment.

### Compound Addition

After baseline activity, cell plates were removed from the FLIPR and transferred to a 384-well pin tool instrument. Prior to compound addition, the pin tool was subjected to 4 wash cycles exposing the pins to water, methanol, and DMSO each time. Compounds, including either “QC compounds” (compounds with known mechanism of action, listed in results) or the mu opioid receptor selective-agonist DAMGO, were transferred from a compound source plate to the 384-well spheroid plate. Immediately after compound transfer, the cell plate was placed back in the FLIPR for the recording that was 1-min after compound transfer to begin. After this 10-min recording, another recording was captured 30-min after compounds were transferred. After the 30-min recording, the plate was again removed from the FLIPR and transferred to the pin tool instrument. During this phase of compound transfer, naloxone was transferred to wells that received DAMGO during the first compound transfer. Following this second compound transfer, the cell plate was placed back in the FLIPR and a final recording was obtained 30-min later, and 70-min after the initial compound transfer.

### Calcium Activity Profiling: Analysis

The FLIPR software ScreenWorks PeakPro 2.0 was used for the initial analysis of calcium activity using automated peak detection settings. With these settings, average peak statistics over each 10-min recording for each well were exported from PeakPro to Microsoft Excel. ScreenWorks Peak Pro 2.0 software was used to extract 17 activity features describing different aspects of calcium activity profiles. These included peak amplitude (PkA), peak amplitude standard deviation (PkASD), peak count (PkCt), peak rate (measured as peaks per minute, PpM), peak rate SD, peak spacing (PkSp), peak spacing SD (PkSpSD), peak spacing regularity, area under the curve (AUC), AUC SD, peak rise slope, peak rise slope SD, peak rise time (PkRt), peak ride time SD (PkRtSD), peak decay slope, peak decay slope SD, peak decay time (PkDt), and peak decay time SD (PkDtSD). To assess variability of these peak parameters, percent coefficients of variance (CV) were calculated by dividing the standard deviation/mean and multiplying by 100 for each designer spheroid type, and those with % CV's below 20% were removed from future analysis. To normalize the data, averages of DMSO-containing wells for each designer spheroid type (e.g., VTA-like vs PFC-like) were obtained for each parameter, and the percent change from DMSO average was calculated for all wells on that plate.

### Statistical Analysis

Normalized FLIPR data obtained from Peak Pro 2.0 was analyzed using both R Studio and TIBCO Spotfire's High Content Profiler (HCP) platform. To analyze calcium activity profiles between different cell-type compositions of designer spheroids, TIBCO Spotfire's High Content Profiler (HCP) was run using standard settings, including principle component analysis (PCA) data exploration, self-organizing map (SOM) class discovery, and z-prime robust for feature selection. Given that data had previously been normalized to DMSO-treated wells in Microsoft Excel, data was not further normalized for the HCP analysis. Only Peak Pro statistics values that were below the coefficient of variance (CV) cutoff (20%) for each plate were used for the HCP analysis and, therefore, SD values for each peak parameter were excluded from analysis due to high variability. To analyze effects of QC compounds on calcium activity, linear mixed model (LMM) ANOVA was used to assess treatment×time interactions, where treatment was the between subjects factor and time was the within subjects factor. One way between subjects ANOVA was performed in R to compare baseline activity changes after chronic DAMGO treatment or DAMGO withdrawal (DAMGO+WD) to DMSO controls. Three-way LMM ANOVA was used to analyze the effect of acute DAMGO treatment on control spheroids as well as spheroids previously treated with chronic DAMGO or the DAMGO+WD group. Here, treatment and group were the between subjects factors and time was the within subjects factor. Significant interactions and main effects were followed up with Tukey's post hoc test and significance was set at p<0.05. In R, the aov function was used for one way ANOVA while nlme was used for 2- and 3-way ANOVAs. The package, Ismeans, was used for post hoc tests. Graphs were created using either GraphPad Prism or TIBCO Spotfire.

### Neurotransmitter Release

Spheroid supernatants were collected and frozen at −80° C. Neurotransmitters dopamine and GABA were measured using a customized LC-MS protocol on HPLC-QQQ/MS-Agilent 6470.

### Microscopy—Clearing and Expansion Microscopy

Spheroids were fixed after 3 weeks culture with 4% paraformaldehyde for 30 minutes, prior to rinsing with 1× phosphate buffered saline (PBS) to remove all traces of paraformaldehyde. Spheroids were permeabilized for 15 minutes with PBS containing 0.3% triton-X-100, then blocked using PBTG (0.1% Triton X-100@ (nonionic surfactant), 5% normal goat serum, 0.1% bovine serum albumin, 1×PBS) for 1 hour at room temperature or overnight at 4° C. Spheroids were then incubated with primary antibodies for general or specific neuronal subtype, astrocytes, and synaptic markers for 2 days overnight at 4° C., followed by extensive washing and staining with secondary antibodies/Hoechst. Spheroids were then cleared using ScaleS4 or equivalent clearing protocol or expanded in order to view synapses.

### Example 2

### Formation of Uniform and Functional, Customizable Spheroids from Fully Matured, IPSC-Derived Neurons and IPSC Derived Astrocyte

### Results

As shown in FIG. 1 the spheroids of different composition of iPSC-derived dopaminergic neurons, GABAergic neurons, glutamatergic neurons, and astrocytes all formed viable spheroids using our internally developed formation protocol. See also FIG. 3. FIG. 2, panel A shows images showing 2 weeks post formation. Each column is a different ratio of iPSC derived neurons (dopaminergic:GABAergic:glutamatergic:10% astrocytes). As shown FIG. 6, shows a general flow-chart of how the spheroids are made.

Spheroids Formed from Single Subtype iPSC-Derived Matured Neurons Exhibit Distinct Calcium Activity Profiles According to Neuronal Type.

As shown in FIG. 11, panels A-C distinct calcium activity profiles of spheroids formed from only (a) iPSC-derived matured dopaminergic neurons+10% astrocytes, (b) iPSC-derived matured GABAergic neurons+10% astrocytes, or (c) iPSC-derived matured glutamatergic neurons+10% astrocytes. These profiles clearly reflect the successful maintenance and maturation of the neurons into a well-synapsed spheroid capable of coordinated firing.

Spheroids of Differing Composition Exhibit Functionally Different Calcium Activity Profiles Corresponding with Input Neuronal Identity

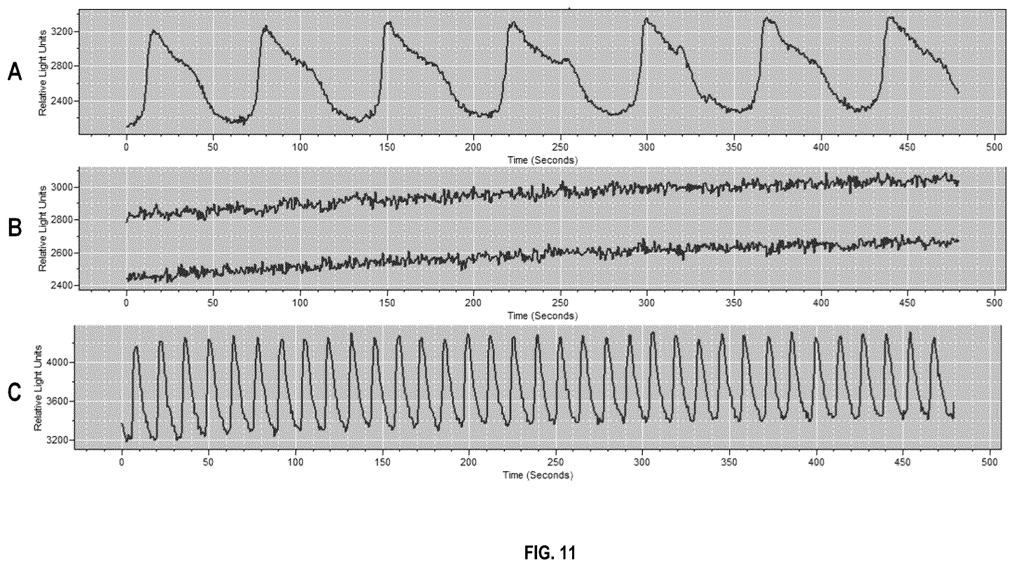

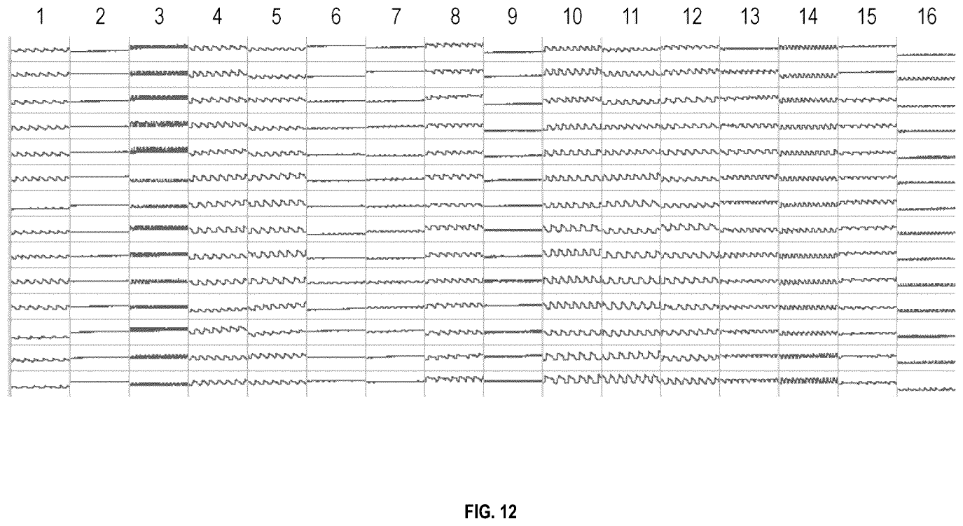

As shown in FIG. 4 384 well plate with spheroids containing 10,000 cells per spheroid of varying composition (see table in materials and methods detailing 16-compositions). The ratios of dopaminergic, GABAergic, and glutamatergic neurons were varied per spheroid with a constant 10% astrocyte incorporation. Shown here are a distinct composition per column, demonstrating the reproducible modulation of activity as a result of changes in ratios of neuronal subtypes incorporated per spheroid. FIG. 4 shows representative wells per column, with the composition shown in a schematic next to each calcium activity plot. Whole plate reading is shown in FIG. 12.

### Formation of Ventral Tegmental Area (VTA) Like and Prefrontal Cortex (PFC) Like Spheroids

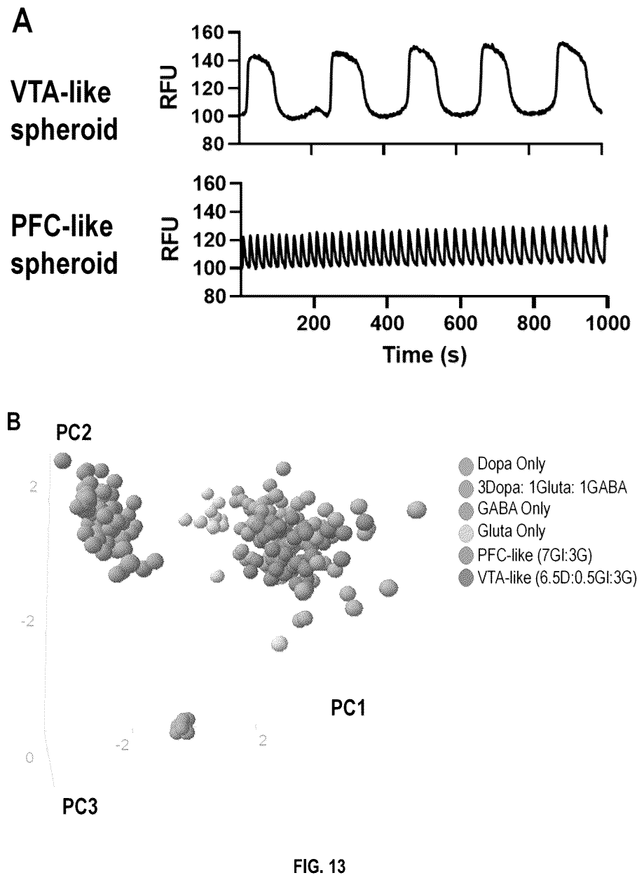

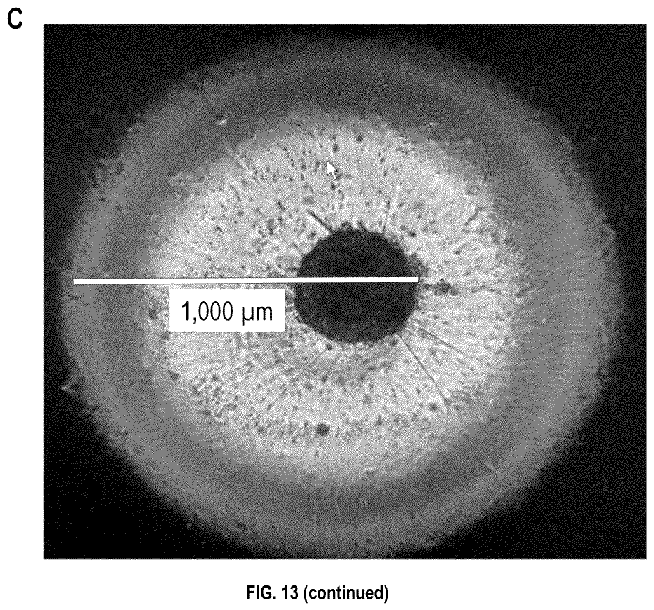

VTA-like and PFC-like spheroids. To make a VTA-like spheroid, spheroids from a neuron ratio of 65% dopaminergic, 30% GABAergic, and 5% glutamatergic neurons with supporting astrocytes for VTA-like spheroids, and 30% GABAergic neurons+70% glutamatergic neurons with supporting astrocytes for PFC-like spheroids. As shown in FIG. 5, PFC-like spheroids show matured activity profiles after only 3 weeks of maturation, compared to a 12-week-old, standard cortical spheroid. FIG. 13, panels A-B shows the distinct calcium activity profiles between VTA-like spheroids and PFC-like spheroids.

### High Content Profiler Analysis Reveals Distinct Activity Profiles Between VTA-Like and PFC-Like Spheroids

Prior to analysis, baseline activity of spheroids was recorded over a 10-min period on a FLIPR Penta by measuring fluctuations in calcium fluorescence. Here, PkA, PkCt, PpM, PkSp, decay slope, and PkDt were reliable measures of calcium activity, since they had coefficient of variations (CV)'s below 20%, and pursued these features when running the high content profiler analysis.

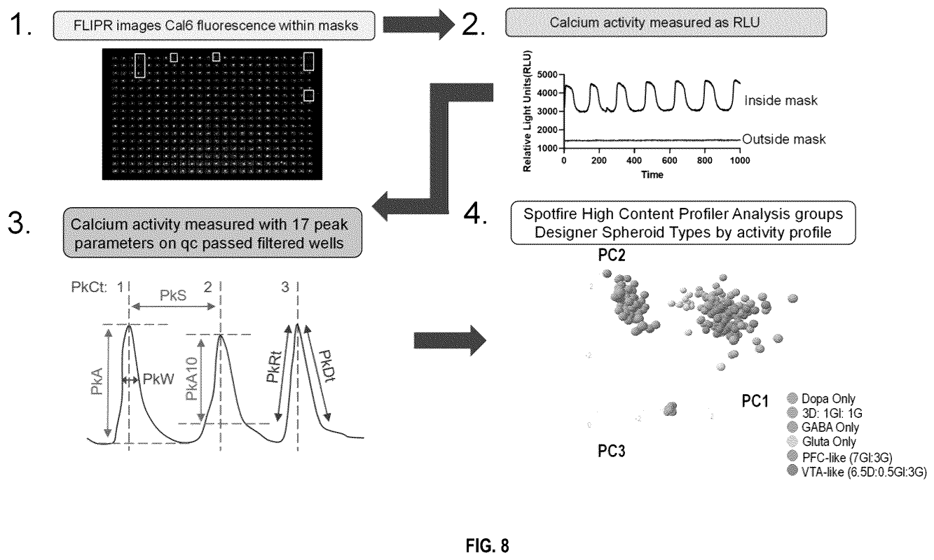

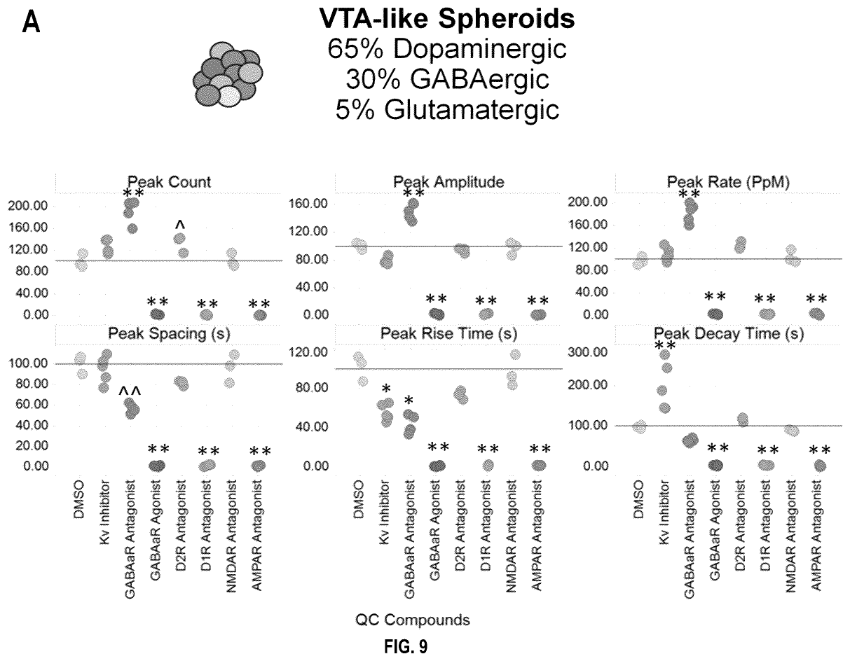

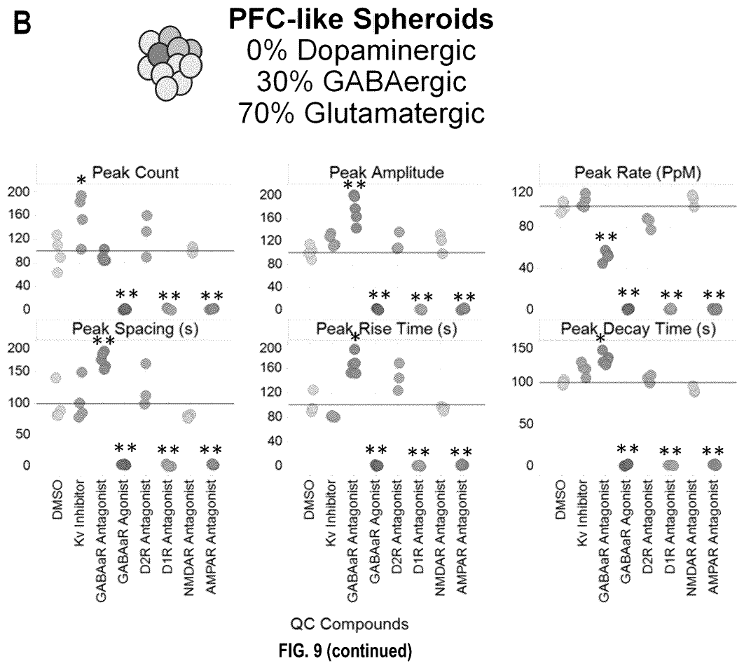

For the high content profiler (HCP) analysis, TIBCO Spotfire was run under standard settings with data normalized to negative control, DMSO-treated, wells. The calcium activity peak parameters with CVs below 20% mentioned above were activity features included in the HCP analysis. Spotfire HCP was run using standard settings, including principal component analysis (PCA) data exploration, self-organizing map (SOM) class discovery, and z-prime robust for feature selection. Data acquisition pipeline is shown in FIG. 8. PCA was created and colored by designer spheroid type, which included control spheroids consisting of dopaminergic neurons only (Dopa Only), glutamatergic neurons only (Gluta Only), GABAergic neurons only (GABA Only), along with PFC-like spheroids and VTA-like spheroids. Importantly, Dopa Only, GABA only, and Gluta Only spheroids showed distinct activity profiles that clustered separately from each other. FIG. 13, panel B. Furthermore, PFC-like spheroids, which consist of GABAergic and glutamatergic, but not dopaminergic neurons, showed activity profiles that clustered in between the glutamatergic Only and GABAergic Only spheroid types. FIG. 13, panel B. Additionally, VTA-like spheroids, which consisted of all three neuron subtypes but with dopaminergic neurons being the dominant neuron subtype, clustered closest to the spheroids that were Dopa Only. Together, this data shows that the baseline activity of PFC-like and VTA-like spheroids are distinct and different from one another, confirming that changing the compositions of neuron subtypes in a controlled manner results in different activity profiles. FIG. 13, panel C shows a VTA-like spheroid by brightfield microscopy.

Validation of VTA-Like and PFC-Like Spheroids with QC Compounds

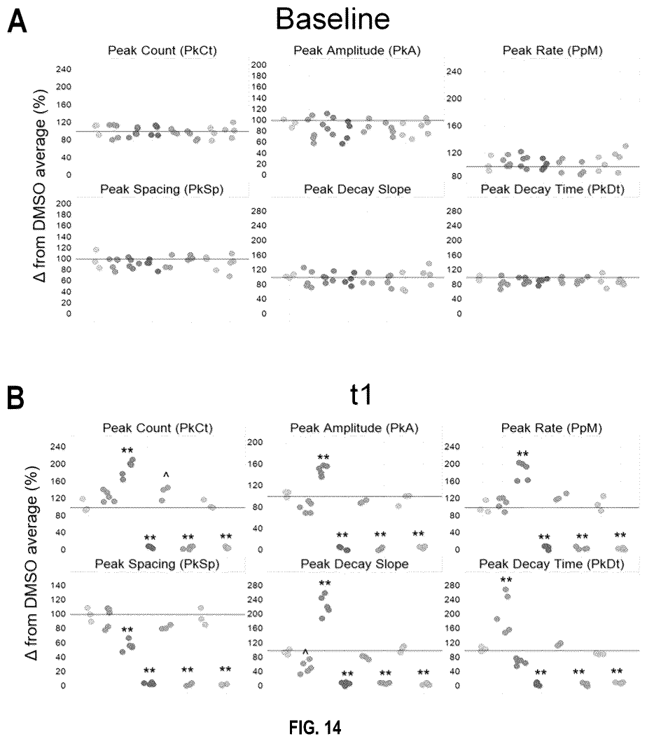

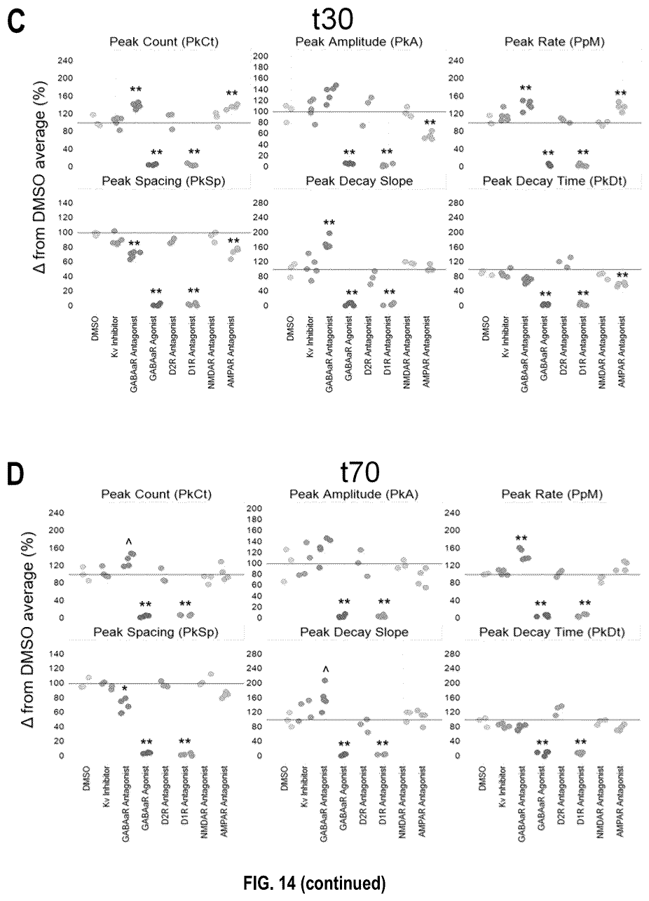

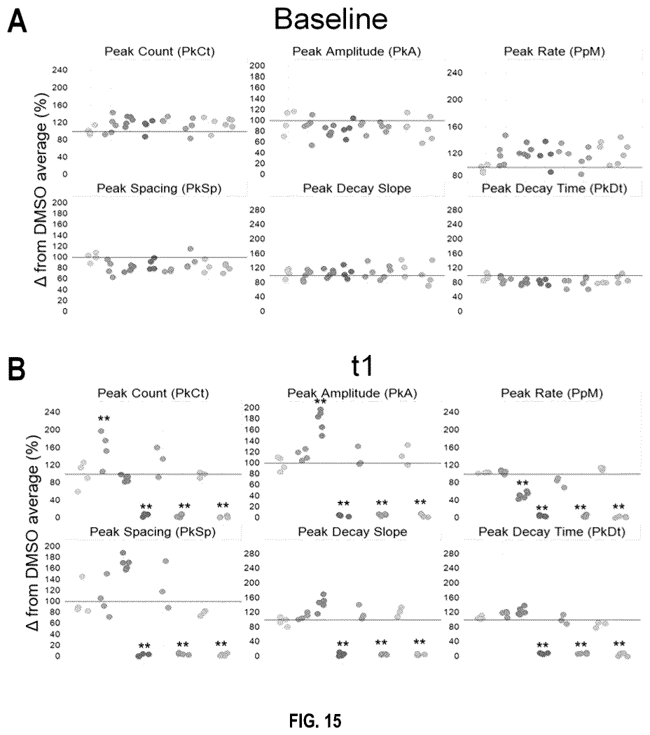

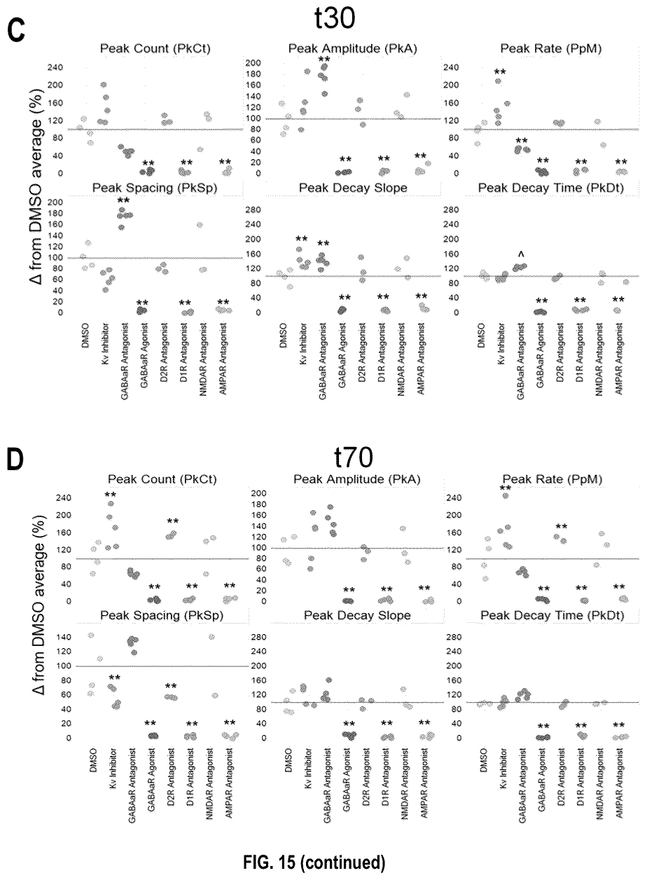

To validate receptor functionality within designer spheroids, VTA-like and PFC-like spheroids were exposed to QC compounds and calcium activity changes were imaged on the FLIPR. FIG. 14 (A-D); FIG. 15 (A-D). Here, 10-min recordings were captured prior to compound exposure (baseline), along with 1-, 30-, and 70-min after compound exposure (t1, t30, t70) and the average of each peak parameter measured (PkCt, PkA, etc.) across the 10-min recording was used for analysis. For analysis, linear mixed model ANOVA was used to examine the effects of compound treatment over the course of the 4 FLIPR recordings (time). Separate 2-way ANOVAs were run for VTA- and PFC-like spheroids and for each peak parameter, and significant treatment×time interactions were observed for each peak parameter (p<0.0001). Therefore, Tukey's post hoc was used for multiple comparisons testing to compare each compound's activity to DMSO for each FLIPR recording, which was used as the vehicle control. As a negative control, a subset of spheroids were exposed to either water or no compound at all to examine any effects of DMSO itself on activity, and no significant differences were found here when looking at the peak parameters analyzed across all four of the FLIPR recordings (PkCt, PkA, PpM, PkSp, Decay slope, or PkDt, p>0.05).

As shown in FIG. 14 (A), the analysis on the baseline recordings for VTA-like spheroids, when all spheroids were under similar conditions before exposure to compound, showed no significant differences between groups, suggesting similar activity profiles for the parameters measured (p>0.05).

4-aminopyradine (4-AP), a voltage-gated potassium inhibitor known to increase action potentials within cortical neurons, was chosen as a positive control for PFC-like spheroids (Wenzel et al., 2017 Cell Reports 19(13): 2681-2693). In VTA-like spheroids, 4-AP did not have any impact on PkCt, PkA, PpM, or PkSp at t1, t30, or t70 (p>0.05). FIG. 14 (B-D). However, 4-AP significantly reduced PkDt at t1 and there was a trend toward statistical significance for increasing the decay slope, though this effect dissipated for recordings 30- and 70-min after treatment (t1, decay slope, p=0.06, PkDt, p<0.0001). FIG. 14 (B-D). In PFC-like spheroids, 4-AP significantly increased PkCt 1-min after exposure (t1: p=0.01). FIG. 15 (B). For the t30 recording, there was a significant increase in peaks per minute (PpM) and the decay slope (t30, PpM: p=0.007, decay slope: p=0.029). FIG. 15 (C) Significant increases in PkCt and PpM as well as a reduction in PkSp resulted from 4-AP 70-min after exposure (t70, PkCt: p=0.015, PpM: p=0.0002, PkSp: p=0.049). FIG. 15 (D). Together, these results suggest that, in VTA-like spheroids, 4-AP only impacts PkDt and decay slope but that these effects were transient since they were not present by t30 and t70. In PFC-like spheroids, however, several activity features were affected by 4-AP which indicated enhanced calcium activity (e.g., increases in PkCt and PpM but decreases in PkSp), with effects most apparent in the t70 recording. These results also highlight temporal differences in 4-AP activity responses between the different designer spheroid types.

The GABA_{A} receptor (GABAaR) agonist, muscimol, and the GABAaR antagonist, bicuculline, were chosen to investigate GABARs within designer spheroids. Muscimol, in both VTA-like and PFC-like spheroids, led to total inhibition of calcium activity that lasted throughout the entire recording period. As such, muscimol treatment caused significant reductions in PkA, PkCt, PpM, PkSp, decay slope, and PkDt compared to DMSO controls at t1, t30, and t70 (p<0.0001). FIG. 14 (A-D); FIG. 15 (A-D). In VTA-like spheroids, GABAaR antagonism with bicuculline increased PkCt, PpM, and decay slope while reducing PkSp, an effect that persisted through the t70 recording (PkCt, t1: p=0.0001, t30: p=0.013, t70: p=0.08; PpM, t1: p=0.0001, t30: p=0.005, t70: p=0.009; PkSp, t1: p=0.0001, t30: p=0.0095, t70: p=0.013; Peak decay slope, t1: p=0.0001, t30: p=0.0007, t70: p=0.04). FIG. 14 (A-D). Additionally, bicuculline increased PkA at t1, though this effect was not present when calcium activity was measured at t30 and t70 (t1: p=0.008). FIG. 14 (B-D). Similar to VTA-like spheroids, bicuculline treatment increased PkA 1- and 30-min after treatment, and there was a trend toward statistical significance 70-min after treatment (PkA, t1: p=0.0001, t30: p=0.0001, t70: p=0.07) (FIG. 14 (B-D)). In contrast to VTA-like spheroids, bicuculline treatment in PFC-like spheroids had no effect on PkCt, though a reduction in PpM and increases in PkSp were observed at t1 and t30 (PpM, t1: p=0.004, t30: p=0.007; PkSp, t1: p=0.0007, t30: p=0.0002). Additionally, PkDt was increased by bicuculline at t1 and 30 but not t70, while peak decay slope was increased only at t30 (PkDt, t1: p=0.01, t30: p=0.08; peak decay slope, t30: p=0.03) (FIG. 15 (B-D)).

Together, this data shows that VTA- and PFC-like spheroids similarly respond to GABAaR agonism, which induced long-lasting inhibition. In VTA-like spheroids, GABAaR antagonism has stimulatory effects on calcium activity, through increases in PkCt, PpM, and decay slope and reductions in PkSp, that persist throughout the entire recording period. FIG. 14 (B-D). Surprisingly, GABAaR antagonism had differentially affected PFC-like spheroids through increases in parameters such as PkA and PkDt that were transient since they were no different from DMSO controls 70-min after treatment. FIG. 15 (A-D).

To assess glutamate receptors, CNQX, an AMPAR antagonist, along with AP5, an NMDAR antagonist, were used. In VTA-like spheroids (FIG. 14), CNQX led to a total inhibition of calcium activity during the t1 recording (FIG. 14 (B)) and, therefore, cause significant reductions in across all parameters compared to DMSO controls (PkA, p<0.0001; PkCt, p<0.0001; PpM, p<0.0001; PkSp, p<0.0001; peak decay slope, p<0.0001; PkDt, p<0.0001). Interestingly, 30-min after CNQX treatment, PkCt and PpM were significantly increased while PkA, PkSp, and PkDt were all still reduced compared to DMSO controls (PkCt, p=0.013; PpM, p=0.008; PkA, p=0.02; PkSp, p=0.009; PkDt, t30: p=0.014). FIG. 14 (C). This data suggests a rebound in calcium activity 30-min after AMPAR blockade in VTA-like spheroids. In support of this, CNQX had no impact on calcium activity in VTA-like spheroids during the t70 recording. This was in contrast to PFC-like spheroids, where CNQX exposure led to a total inhibition of calcium activity that lasted throughout the entire recording period (t1-t70: p<0.0001). FIG. 15 (A-D). Treatment with AP5 did not impact calcium activity in either VTA- or PFC-like spheroids, and future studies plan to repeat this using a higher dose. FIG. 14-15.

These results suggest that AMPAR blockade produces inhibition of calcium activity for a longer-lasting period in PFC-like spheroids, which contain 70% glutamatergic neurons, compared to VTA-like spheroids, which contain only 5% glutamatergic neurons. These results highlight how spheroid neuronal subtype composition can impact response to pharmacological manipulation.

Since dopamine 1 receptors (D1Rs) are stimulatory G-protein coupled receptors while D2Rs are inhibitory G-protein coupled receptors, antagonists were used targeting each of these to investigate whether they would have opposite effects on calcium activity. SCH23390 was used to block D1 Rs and sulpiride to block D2Rs. In both VTA- (FIG. 14) and PFC-like (FIG. 15) spheroids, blocking D1 Rs led to inhibited calcium activity that lasted throughout the entire recording period and therefore, significant reductions in all peak parameters were observed compared to DMSO controls (p<0.0001). FIG. 14 (A-D); FIG. 15 (A-D). In VTA-like spheroids, sulpiride treatment did not impact calcium activity, though there was a statistical trend toward significantly increasing PkCt 1-min after treatment (t1: p=0.08). FIG. 14 (B). In PFC-like spheroids, however, sulpiride significantly increased PkCt and PpM while decreasing PkSp 70-min after treatment compared to DMSO controls (PkCt, t70: p=0.03; PpM, t70: p=0.0014; PkSp, t70: p<0.0001). FIG. 15 (A-D).

These results suggest that D2Rs may be more highly expressed on glutamatergic neurons, which are in higher prevalence in PFC-like spheroids. Furthermore, sulpiride's effects are delayed, with differences not observed until 70-min after compound exposure (FIG. 15 (D)).

### Effects of Chronic DAMGO Treatment and DAMGO Withdrawal on Baseline Calcium Activity

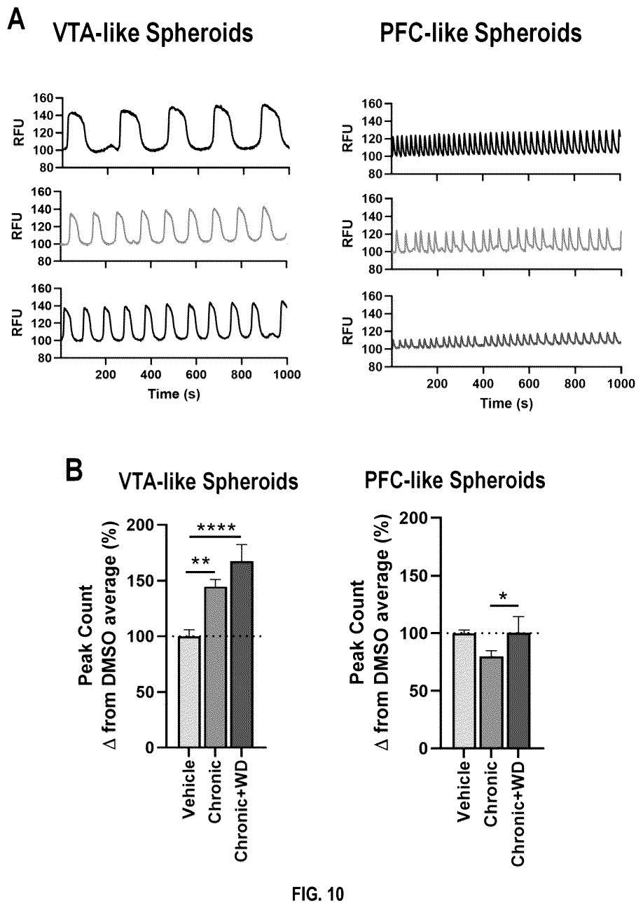

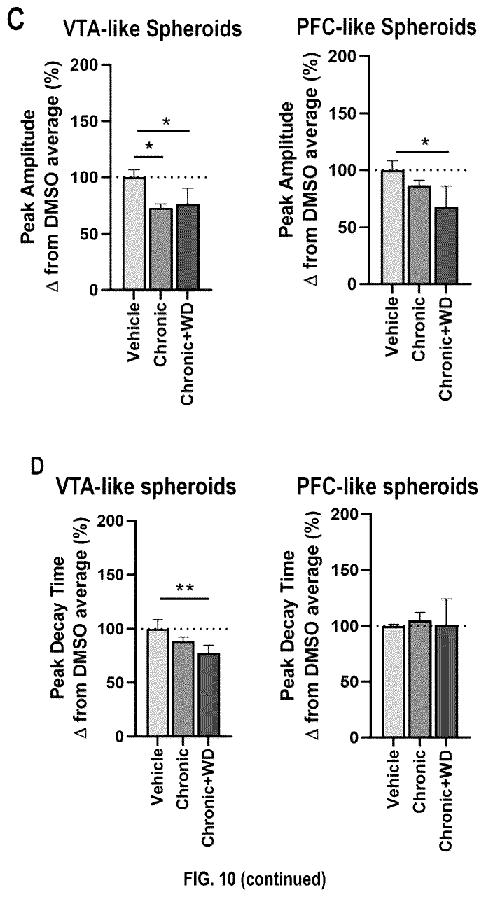

To examine the effects of both chronic DAMGO treatment along with 3-days DAMGO withdrawal on baseline activity of spheroids, one way ANOVA was used to compare these two groups to DMSO controls. Here, PkCt, PkA, and PkDt were assessed within each designer spheroid type since each of these peak parameters showed significant main effects of treatment (p<0.05). Within VTA-like spheroids, both chronic DAMGO treatment and DAMGO withdrawal significantly increased PkCt while decreasing PkA baseline activity (PkCt, Chronic: p=0.003, Chronic+WD: p<0.0001; PkA, Chronic: p=0.019, Chronic+WD: p=0.034). FIG. 10 (A-C). However, only DAMGO withdrawal led to a significant reduction in PkDt compared to controls (p=0.016). FIG. 10 (A & D). In PFC-like spheroids, neither chronic DAMGO nor DAMGO withdrawal led to significant differences in PkCt compared to controls, but PkCt in spheroids chronically treated with DAMGO was lower than in spheroids undergoing DAMGO withdrawal (p=0.029). FIG. 10 (A-B). For PkA, only DAMGO withdrawal led to a significant reduction compared to controls (FIG. 10 (A & C), and neither chronic DAMGO nor DAMGO withdrawal impacted baseline PkDt in PFC-like spheroids (DAMGO+WD: p=0.019). FIG. 10 (A & D).

### Chronic DAMGO Treatment and DAMGO Withdrawal

On day 11, spheroids began receiving chronic DAMGO. DAMGO (Tocris, 1171) was reconstituted in water to a 10 mM stock solution. On days with media changes, DAMGO was added to media to make a 20 μM solution. Since half media changes were done every other day to maintain spheroids, the final dose of DAMGO treatment for these spheroids was 10 μM. Chronic DAMGO-treated spheroids were treated with 10 μM DAMGO each time they received a media change, every other day, until day 21 when FLIPR recordings took place. As such, they received 5 total treatments overall. To model withdrawal and forced drug abstinence, a subset of these spheroids were assigned to a group receiving chronic DAMGO but undergoing 3-days withdrawal. These spheroids were administered DAMGO under the same conditions as the chronically treated group, but on the final day media was changed before FLIPR recordings, they received media without DAMGO.

Effects of Acute DAMGO Treatment on Control Spheroids and Those Chronically Treated with DAMGO

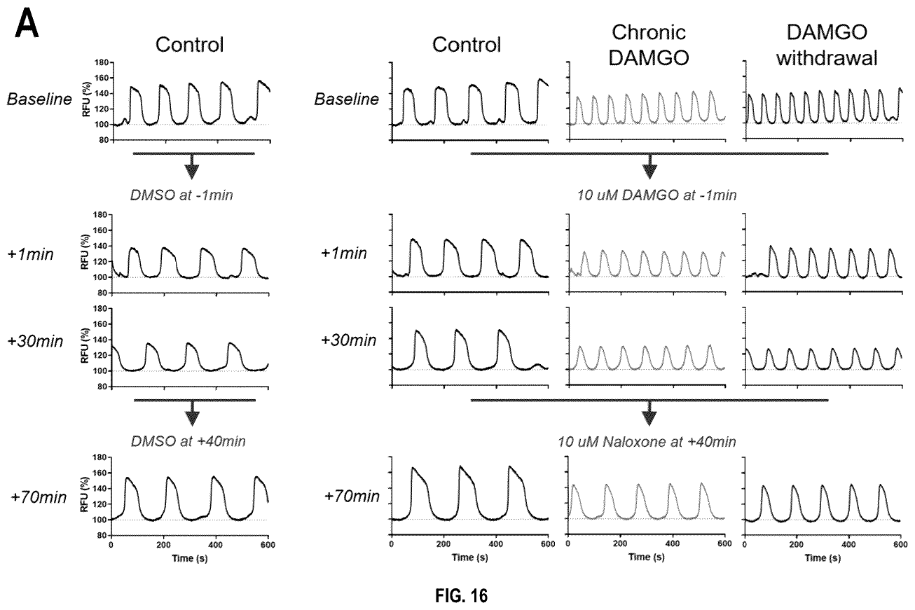

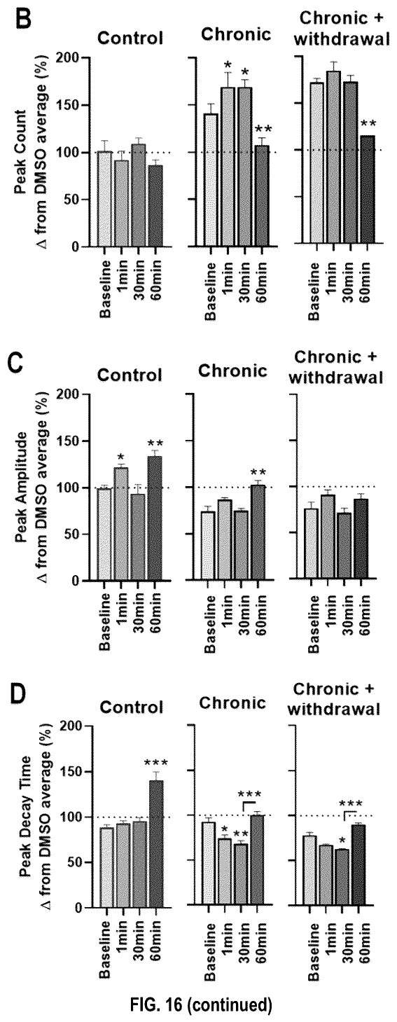

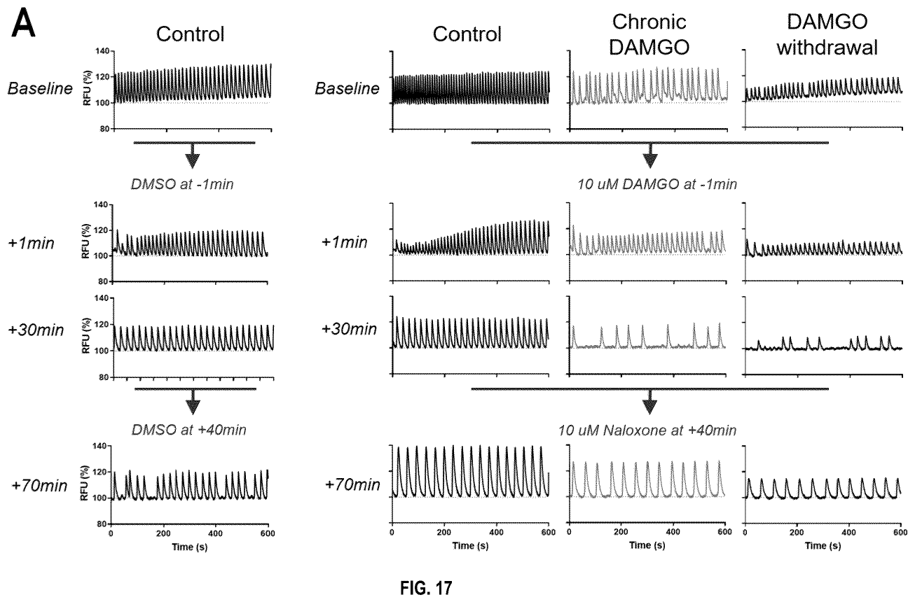

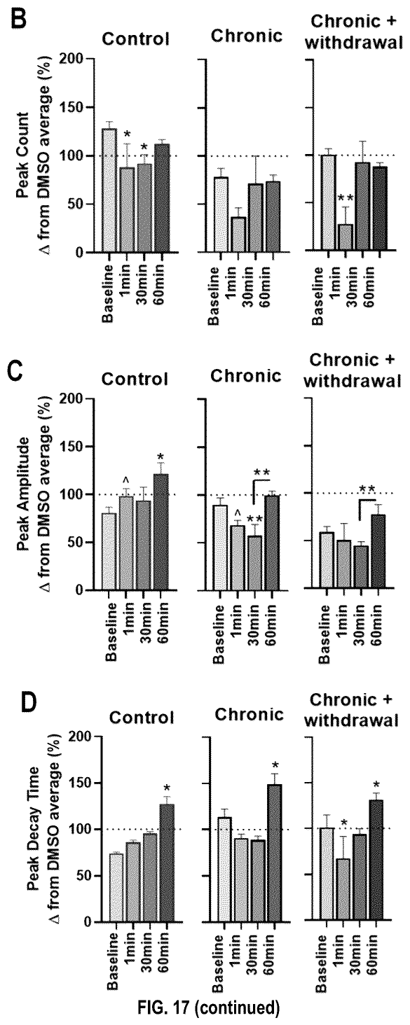

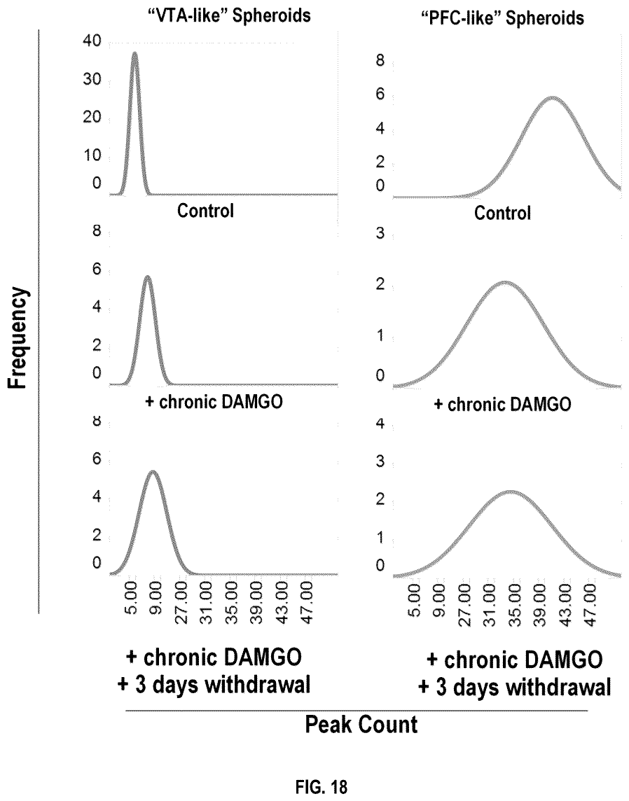

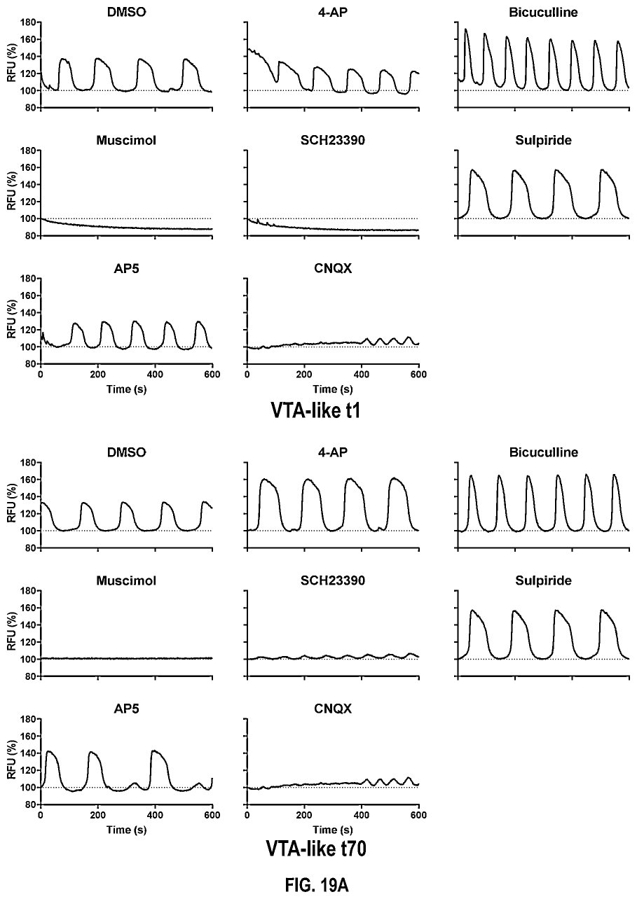

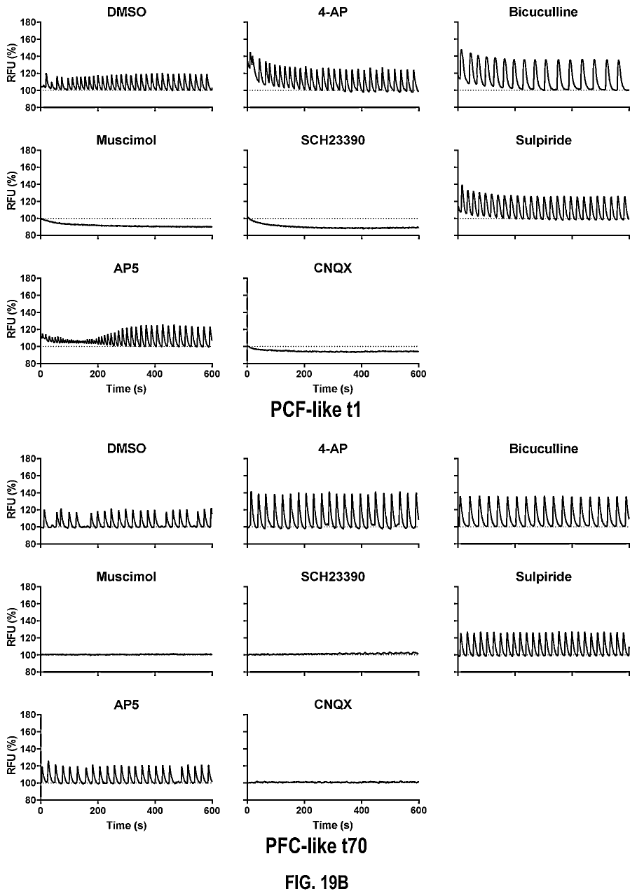

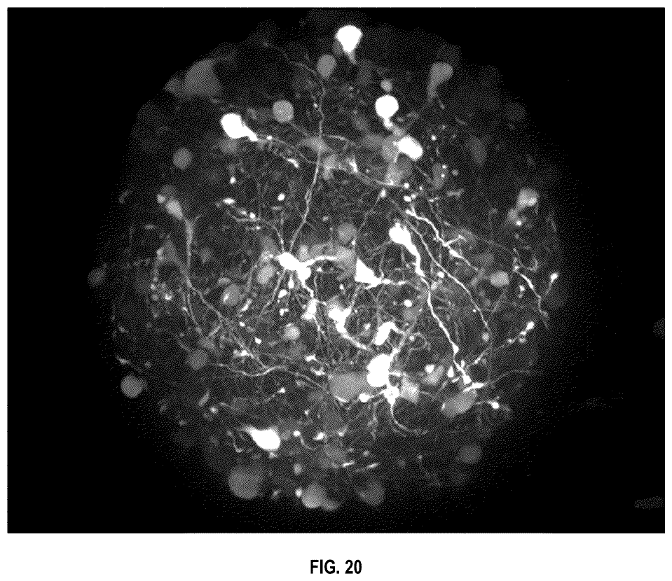

Three-way ANOVAs analyzing the interactions between treatment, group, and time were used to investigate the effect of acute DAMGO treatment on calcium activity within control spheroids, those chronically treated with DAMGO, or those undergoing 3-days DAMGO withdrawal. All data was normalized to DMSO-treated wells within each FLIPR recording measured, and treatment effects were compared to baseline activity within each group. In control VTA spheroids, acute DAMGO had no impact on PkCt (FIG. 16 (B)) or PkDt (FIG. 16 (B)) but did produce a significant increase in PkA in VTA-like spheroids (FIG. 16 (C)). This increase in PkA was only present 1-min after exposure and went away 30-min later (PkA, t1: p=0.012). FIG. 16 (C). In PFC-like spheroids, acute DAMGO treatment reduced PkCt at t1 and t30, (FIG. 17 (A, B)) suggesting an overall inhibitory effect (PkCt, t1: p=0.047, t30: p=0.026).

There was a statistical trend toward an increase in PkA in PFC-like control spheroids in response to acute DAMGO treatment, but it was not significant until t60 (t1: p=0.08) (FIG. 17 (C)). Overall, these results suggest that acute DAMGO treatment in control spheroids has transient effects on activity in VTA-like spheroids and inhibitory effects on calcium activity in PFC-like spheroids. FIGS. 16-17.

In VTA-like spheroids that had been chronically treated with DAMGO, acute DAMGO treatment increased PkCt (FIG. 16 (A, B)) and decreased PkDt (FIG. 16 (D)) at both t1 and t30 compared to baseline (PkCt, t1: p=0.003, t30: p=0.003; PkDt, t1: p=0.0057, t30: p=0.0002). In VTA-like spheroids undergoing DAMGO withdrawal, acute DAMGO treatment did not impact PkCt or PkA but did significantly reduce PkDt 30-min after treatment (p=0.012) (FIG. 16 (B-D)). In PFC-like spheroids, acute DAMGO treatment to spheroids that had been chronically treated with DAMGO did not impact PkCt and PkDt but did reduce PkA 30-min after treatment (t1: p=0.07, t30: p=0.0029) (FIG. 17 (A-D)). In PFC-like spheroids undergoing DAMGO withdrawal, acute DAMGO treatment did not impact PkA but did reduce PkCt and PkDt 1-min, but not 30-min, after treatment (PkCt, t1: p=0.0005; PkDt, t1: p=0.0187). (FIG. 17 (A-D))

This data suggests that acute DAMGO treatment in VTA-like spheroids chronically treated with DAMGO increases spheroid activity (through increased PkCt and decreased PkDt). Similar findings are observed for VTA-like spheroids undergoing DAMGO withdrawal, though the effects are not as pronounced as chronically treated DAMGO spheroids since their baseline activity was significantly higher than chronic DAMGO and control groups. Furthermore, acute DAMGO treatment in PFC-like spheroids to chronic DAMGO and DAMGO withdrawal groups has inhibitory effects that are observed through reductions in PkA.

### Effect of Naloxone Treatment on Reversing DAMGO-Induced Deficits in Calcium Activity

In control spheroids that had been acutely treated with DAMGO, naloxone increased both PkA and PkDt 30-min after treatment (VTA-like: PkDt, t70: p=0.0001; PkA, t70: p=0.0001; PFC-like: PkDt, t70: p=0.0001; PkA, t70: p=0.0001). FIG. 16 (C-D). In chronic DAMGO-treated VTA-like spheroids, naloxone rescued deficits in PkCt, PkA, and PkDt, bringing these activity features back to the level of DMSO controls (PkCt: p=0.0004; PkA: p=0.0047; PkDt: p<0.0001). FIG. 16 (B-D). In VTA-like spheroids undergoing DAMGO withdrawal, naloxone rescued deficits in both PkCt and PkDt to the level of DMSO controls (PkCt: p<0.0001; PkDt: p<0.0001). FIG. 16 (B, D). In PFC-like spheroids chronically treated with DAMGO and undergoing DAMGO withdrawal, naloxone had no effect on PkCt since the deficits induced by acute DAMGO treatment were transient (p>0.05). FIG. 17 (B).

Additionally, naloxone increased PkDt in both chronic DAMGO groups compared to their baseline PkDt (Chronic: p=0.03; Chronic+WD: p=0.038). However, naloxone treatment rescued PkA deficits induced by acute DAMGO treatment within PFC-like spheroids to the level of DMSO controls (Chronic: p<0.0001; Chronic+WD: p<0.0001). FIG. 17 (C, D).

Overall, this data shows that naloxone rescues the deficits induced by acute DAMGO treatment in both chronic DAMGO-treated and DAMGO withdrawal VTA-like spheroids. In PFC-like spheroids, deficits in PkA that were observed by acute DAMGO treatment were rescued by naloxone.

### Discussion

The baseline changes after chronic DAMGO treatment and DAMGO withdrawal in VTA-like spheroids are consistent with data from in vivo animal and human studies, showing increased basal activity within this brain region. Human studies show elevated BOLD signal activation within the VTA of people suffering from heroin addiction in response to visual cues related to heroin, when compared to the VTA of healthy individuals (Yang et al., 2009; Zijlstra et al., 2008). In animal studies, chronic morphine exposure in mice increases the basal firing rate of dopaminergic neurons in the VTA, measured by patch clamp electrophysiology (Koo et al., 2012). This is in line with findings also showing that 3-days withdrawal from chronic morphine leads to a constitutively active state of mu opioid receptors, inhibitory G-protein coupled receptors on GABAergic VTA neurons, leading to a disinhibition of dopamine neurons within this brain region (Meye et al., 2014). The Meye et al. study also uses electrophysiology to show increased inhibition of GABAergic neurons in the VTA by measuring miniature inhibitory post synaptic currents (mIPSCs, Meye et al., 2014).

The results show an overall inhibitory effect from chronic DAMGO treatment and DAMGO withdrawal in basal activity in PFC-like spheroids across measures such as peak amplitude. Similar to findings in the VTA, human studies show elevated BOLD signal activation within the frontal cortex (Langelben et al., 2008). However, it should be noted that BOLD signal intensity does not necessarily mean increased excitatory activity, as it measures blood flow to a brain region, and therefore could still indicate increased activity of inhibitory GABAergic neurons. For instance, animal studies similarly show increased MRI signal activation within the prefrontal cortex of mice chronically treated with morphine, but in vivo electrophysiology data suggests heroin self-administration inhibits prefrontal cortical neurons (Chang et al., 1997; Niu et al., 2017). Furthermore, chronic morphine is associated with dendritic spine loss within glutamatergic pyramidal neurons within the PFC, suggesting reduced glutamatergic transmission and enhanced inhibition within this brain region (Robinson and Kolb, 1999).

It is also worth noting that blocking mu opioid receptors (MORs), in inhibitory G-protein coupled receptors, in human studies reduces BOLD signal activation increases observed in the VTA and PFC of people with heroin addiction (Langleben et al., 2008; Mei et al., 2008). This is in line with our data showing that naloxone, a mu opioid receptor antagonist used to reverse opioid overdose in humans, rescues deficits induced by acute DAMGO exposure in spheroids chronically treated with DAMGO or undergoing DAMGO withdrawal.

### Example 3

### Generation and Maintenance of Neurodegenerative Disorder Model Spheroids

Cells and Donor Information: Matured, differentiated iPSC-derived cells were obtained from FujiFilm CDI. Wildtype (Wt) cells included iCell DopaNeurons (cat #R1088), iCell GlutaNeurons (cat #R1061), iCell GABANeurons (cat #R1013) and iCell Astrocytes (cat #R1092). iCell Dopa Neurons PD SNCA A53T HZ (cat #R1109) were used to model Parkinson's Disease (PD) while iCell GABANeurons (APOE e4/4) (cat #R1168) were used to model Alzheimer's Disease. The donor ID for Wt iCell DopaNeurons along with iCell GlutaNeurons was 01279, a healthy male age 50-59; the donor ID for A53T iCell DopaNeurons was also 01279 and was genetically engineered to have the SNCA A53T mutation. The donor ID for iCell Astrocytes as well as Wt and APOE iCell GABANeurons was 01434, a healthy female<18 years old, with the APOE e4/4 line being an engineered line.

Tissue Culture Media: After each cell type was thawed, base media with supplements was used to create a cell suspension. Base media used to form spheroids differed by cell type; iCell Base Medium 1 (CDI, #M1010) was used for iCell DopaNeurons, iCell GABANeurons, and iCell Astrocytes while BrainPhys Neuronal Medium (Stem Cell Technologies, #05790) was used for iCell GlutaNeurons. Supplements and iCell Base Medium 1 were provided in the iCell kits referenced above, and 2% Neural Supplement B plus 1% Nervous System Supplement were added to media for iCell DopaNeurons, while media for iCell GABANeurons contained 2% Neural Supplement A. Media for iCell GlutaNeurons contained 2% Neural Supplement B, 1% Nervous System Supplement, 1% N2 supplement (Thermo, 17502048), and 0.1% laminin (Invitrogen, #23017-015) in BrainPhys media. Base media for iCell GABANeurons was used for Astrocytes.

The day after spheroids were plated, maintenance media was added such that each well contained 90 μL with 45 μL consisting of base media and 45 μL consisting of maintenance media. Maintenance media was the same for all spheroids. BrainPhys Neuronal Medium was used and supplemented with 1× N2, 1× B27 (Thermo, cat #17504), 20 ng/mL BDNF and 20 ng/mL GDNF (Stem Cell Technologies, cat #78005 and 78058, respectively), 1 μg/mL laminin, 1 mM cAMP (Tocris, cat #1141), and 20 nM ascorbic acid (Tocris, cat #4055). A stock solution consisting of all materials except cAMP and ascorbic acid was prepared in advance, and the cAMP and ascorbic acid were added to the media fresh on each day of media changes. Half media changes occurred every other day, and spheroids were maintained for 3-weeks.

Cell Thawing: For thawing, iCell DopaNeurons (Dopa), iCell GABANeurons (GABA), and iCell Astrocytes (Astro) were placed in a 37° C. water bath for 3 minutes and iCell GlutaNeurons (Gluta) neurons for 2 minutes, according to the manufacturer's instructions. The contents of each vial were dispensed into separate 15 mL conical tubes. Base media (1 mL) for each cell type was added to the empty cell vials to collect any remaining cells, dispensed in drop-wise fashion on top of the cell suspension in each tube, then 8 mL of media was added to each tube. Tubes containing cell suspension of GABA and Astro cells were centrifuged at 300 g×5 minutes, while tubes with either Dopa or Gluta cell suspension were centrifuged at 400 g×5 minutes (min). The supernatant was aspirated and resuspended in 2 mL of base media, then cells for each cell type were counted using a Countess Cell Counter (Thermo).

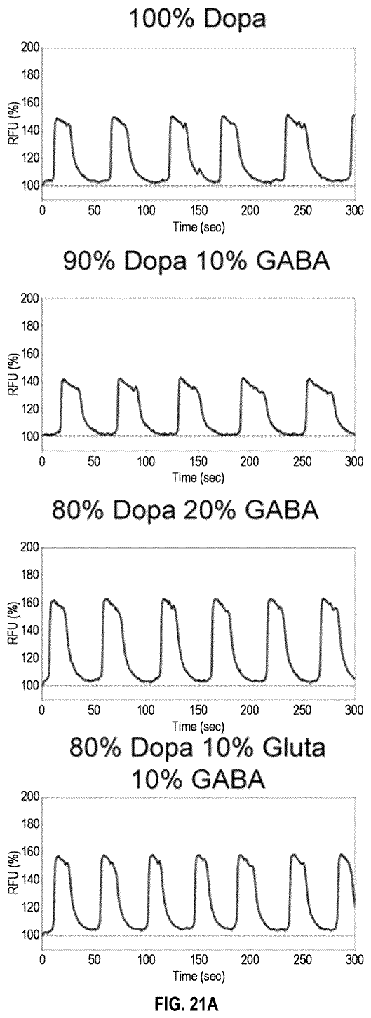

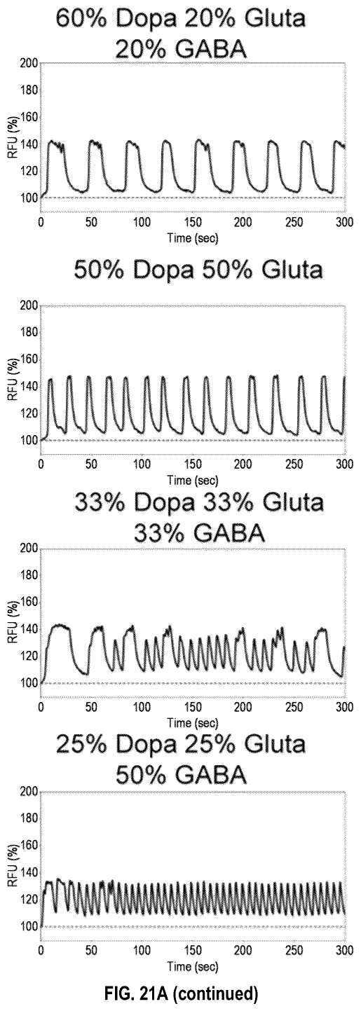

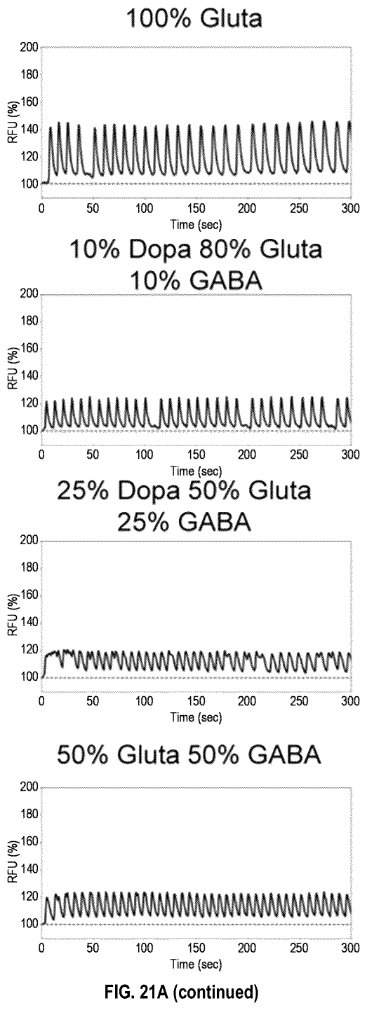

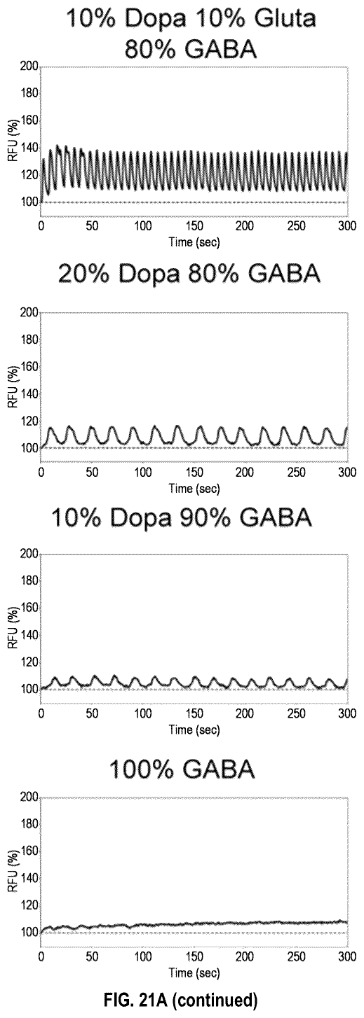

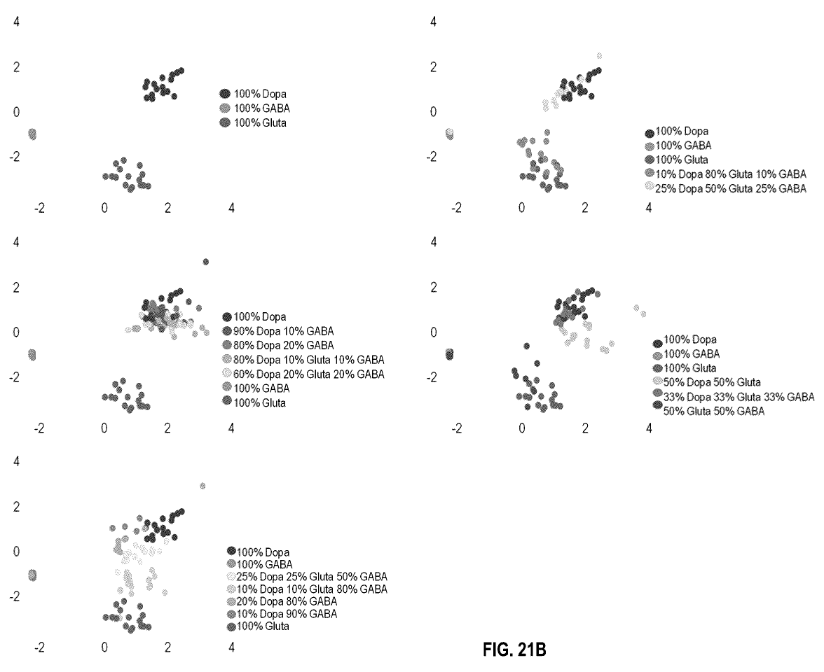

Generation of spheroids: After counting, base media was added to achieve a cell suspension containing 5e5 cells/mL for each cell type. Cell types required in each spheroid type were then mixed in fresh 50 mL conical tubes. The cell type compositions of control spheroids include: 100% dopaminergic (dopa), 100% glutaminergic (gluta), 100% GABAergic (GABA), 90% dopa+10% astrocytes (astro), 90% gluta+10% astro, 90% GABA+10% astro. A study (FIG. 21A, 21B) used 16 randomly generated spheroids consisting of 90% neuron and 10% astrocyte but with differing percentages of neuronal subtypes, and these spheroid compositions, e.g., 100% Dopa; 100% GABA; 100% Glutamergic; 90% Dopa: 10% GABA; 80% Dopa: 20% GABA; 80% Dopa: 10% Glutamergic:10% GABA; 60% Dopamergic:20% Glutamergic:20% GABAergic; 25% Dopamergic:25% Glutamergic:50% GABAergic; 10% Dopamergic:10% glutamergic:80% GABAergic; 20% Dopamergic: 80% GABAergic; 10% Dopamergic:90% GABAergic; 10% dopamergic: 80% Glutamergic: 10% GABAergic; 25% dopamergic: 50% glutamergic: 25% GABAergic; 50% dopamergic:50% glutamergic; 33% dopamergic:33% GABAergic:33% GABAergic: or 50% glutamergic: 50% GABAergic. Brain region-specific spheroids modeling the ventral tegmental area (VTA-like) and prefrontal cortex (PFC-like) each contained 90% neurons+10% astrocytes with differing neuronal cell type compositions. VTA-like spheroids contained 65% dopa, 5% gluta, and 30% GABA neurons while PFC-like spheroids contained 70% gluta+30% GABA neurons. Once a cell suspension was created containing the cell type composition needed, 50 μL was manually dispensed with a 16-channel multichannel Finnpipette (Thermo) into 384-well round bottom ultra-low attachment (ULA) spheroid microplates (Corning, cat #3830). Plates were then sealed with parafilm and centrifuged at 1485×g for 10 minutes (min) to pull cells to the bottom of the plate. One day later, 5 μL of base media was removed and 45 μL maintenance media was added as described herein.

Generation of assembloids: To model neural circuits between brain regions, assembloids were formed with VTA- and PFC-like spheroids. An assembloid can comprise two or more spheroids connected to each other. One week prior to recording, one of each spheroid type were combined into a 1.7 mL tube together. Here, one spheroid type was expressing AAV9-GCaMP6f while the other was expressing either an inhibitory or excitatory DREADDs virus. Both spheroids were pulled up into a wide bore 200 μL pipette tip (Rainin, cat #30389188) with only 15 μL media and dispensed into the bottom of a well int the Corning ULA round bottom plates (#3830). Collagen I (Fisher, cat #CB354249) was made with media, 10× phosphate buffered saline, and 1 N NaOH at a 3 mg/mL concentration and 15 μL was pipetted on top of the two spheroids. The plate was placed in the incubator at 37° C. for overnight gelling and the following day, 50 μL media was added to the wells. Maintenance media used was the same as spheroids and half media changes were performed every other day prior to testing.

### Measurement of Calcium Activity

Viruses and dyes: To assess calcium activity, the calcium dye, Cal6 (Molecular Devices), and genetically encoded calcium indicator, GCaMP6f (Addgene, cat #100836-AAV9) were used. Cal6 was used according to manufacturer's instructions and 10 mL of maintenance media was added to each vial. Two hours before activity was recorded, half of the spheroid media was exchanged for media with Cal6. The plates were covered in foil and placed in the incubator at 37° C. during the 2-hour (hr) incubation period. Since all viruses used were adeno-associated viruses (AAV), they were added to the media on day 7 to allow for 2-weeks expression prior to recording or testing. All viruses were added to the media at 2e5 multiplicity of infection (MOI). GCaMP6f infection occurred via an adeno-associated virus serotype 9 (AAV9) expressed under the CAG promoter for expression in both neurons and astrocytes. Designer receptors exclusively activated by designer drugs (DREADDs) viruses were used to stimulate and inhibit neuronal activity within spheroids. Both viruses were retrograde AAVs expressed under the human Synapsin promoter for expression in neurons and fused with an mCherry fluorophore. The DREADDs viruses either inserted the designer receptor hM4D(Gi), an inhibitory G-protein coupled receptor, or hM3D(Gq), a stimulatory G-protein coupled receptor. Clozapine-N-oxide (CNO, Tocris cat #4936) was suspended in dimethyl sulfoxide (DMSO) and used as the designer drug to activate the DREADDs viruses. CNO was tested at 1 and 10 μM, with data reported from the 1 μM concentration.

Fluorescent Imaging plate Reader (FLIPR): The FLIPR Penta (Molecular Devices) was used to assess calcium fluorescence across the 384-well plate simultaneously and to observe changes after treatment with compounds. The evening before calcium activity was measured, plates were sealed with parafilm and centrifuged at 1485×g for 2-min to get spheroids to the bottom of the well in a centered position. On the day of recording, the plate used for recording was placed in the read plate position inside of the FLIPR following the 2-hour Cal6 incubation at 37° C. Standard filter sets were used for Cal6 imaging with excitation set at 470-495 and emission at 515-575 nm. Fluorescent image reads were taken every 0.6 seconds for all plates, with exposure time of 0.03 and 50% excitation intensity. Recordings from the initial seven plates consisted of 1000 reads and were 10-min recordings with 2.5 gain, while the final two plates consisted of 500 reads (5-min recordings) with a gain of 2. Baseline recordings were taken across all plates and, if applicable, more recordings were obtained 1-, 30-, 60-, and/or 90-min after compound treatment. In between recordings, plates were wrapped in foil and placed back in the incubator at 37° C.

Confocal Imaging: The Opera Phenix Plus High-Content Imaging System (Perkin Elmer) spinning disk confocal was used to record calcium activity from spheroids in individual wells. Prior to recording, the stage was pre-warmed to 37° C., and the carbon dioxide was set to 5%. Recordings were obtained both from spheroids expressing GCaMP6f along with those incubated in Cal6 dye. Recordings were captured with a 20× water immersion objective 55 m from the bottom of the well, and were obtained at a frame rate of 1.6 frames/sec with 480 frames total, making the recordings 5-min. The protocol was set to record well to well such that recordings were automated but taken from one spheroid at a time before recording from subsequent wells. For all calcium activity recordings obtained from the Phenix Plus, the FITC channel was used where excitation was set to 488 nm and emission at 535 nm. For spheroids expressing GCaMP6f, the exposure time was set to 20 millisecond (ms) and the laser power was set to 30% while for spheroids in Cal6 dye, the exposure time was 20 milliseconds (ms) with laser power set to 10%. The focal plane for recordings from assembloids varied depending on where they were suspended in collagen, though these recordings were all obtained within 250 m from the bottom of the well. For assembloids, the exposure time was set to 40 ms with laser power set to 40%.

### Drug Treatment

Chronic DAMGO treatment: To model opioid use disorder (OUD), a subset of spheroids was treated with DAMGO (Tocris, cat #1171), a selective mu opioid receptor (MOR) agonist, chronically during the 3-week spheroid maintenance period. DAMGO was reconstituted in water at a 1 mM concentration and diluted in media to 20 M such that spheroids would be treated with a final concentration of 10 μM after the half media exchange. Two aspects of OUD were modeled, chronic treatment along with opioid withdrawal. For spheroids subjected to chronic DAMGO treatment, 20 μM DAMGO in maintenance media was added via half media exchange beginning on day 10, with treatments occurring every other day for 10 days, giving a total of five treatments. For spheroids subjected to DAMGO withdrawal, the same protocol was followed except that spheroids did not receive DAMGO for the final treatment and instead were subjected to a three day washout period intended to model the withdrawal aspect of OUD.

384 Pin Tool: A 384 well pin tool (Rexroth) was used to transfer compounds simultaneously to the spheroid plate. Prior to compound transfer, the pin tool went through four wash cycles where pins were rinsed with dimethyl sulfoxide (DMSO), followed by methanol, then deionized (DI) water, to ensure the pins were clean. Compound transfer via the 384 well pin tool occurred after the baseline recordings with the fluorescent imaging plate reader (FLIPR). Here, 60 nL of compound suspended in DMSO was transferred to 60 L of media in each well, diluting the compounds by 1000-fold and giving a final DMSO concentration of 0.1%. Immediately after compound transfer, the spheroid plate was either placed back inside of the FLIPR for a recording 1-min after compound treatment or placed back in the incubator if post-treatment recording was >30-min after compound transfer.

### Cell Viability Assays

3D Cell Titer Glo: To measure spheroid cell viability across disease models and after compound treatment, the CellTiter-Glo 3D Cell Viability Assay (Promega, cat #G9681) was used according to the manufacturer's instructions. CellTiter-Glo 3D Reagent was thawed overnight at 4° C. and brought to room temperature (RT) for 20-min before use. After the FLIPR assay, 30 μL of CellTiter-Glo 3D Reagent was added to the spheroid plate and was mixed by shaking for 5-min at RT followed by a 25-min incubation period off the shaker at RT (about 25° C.). Luminescence was read using a PHERAstar FSX microplate reader (BMG LabTech) to measure amount of ATP present, indicating metabolically active cells.

Calcein and Propidium Iodide (PI) staining: Imaging of live and dead cells was done via Calcein (Thermo, cat #C1430) and PI (Thermo, cat #P3566) staining on live spheroids. Calcein AM and PI were diluted in 1× Dulbecco's phosphate-buffered saline (DPBS; Thermo, cat #14040141) to concentrations of 1:2000 and 1:1000 to achieve final concentrations of 0.5 and 1 μM, respectively. Half of the media was removed from each well (45 μL) and was exchanged with Calcein AM and PI in DPBS. Spheroids were incubated at 37° C. for 30-min prior to live cell imaging. For imaging, spheroids were placed in the Phenix Plus with the stage pre-warmed to 37° C. and 5% carbon dioxide circulating. The FITC channel, with 488 nm excitation and 535 nm emission, was used to image Calcein AM while Cy3 (excitation of 530 nm, emission of 620 nm) was used to image PI. 150 μM image stacks were collected with a 10× air objective using a 2 μm z-step.

### Tissue Processing

Spheroid fixation: Spheroids were fixed with 4% paraformaldehyde (PFA) in PBS overnight at 4° C. The following day, spheroids were washed with PBS, where half of the PFA was removed and exchanged with PBS, a total of four times. On the final wash, PBS with 0.1% sodium azide (Sigma, cat #S2002) was added for spheroid preservation. Plates were sealed with parafilm and stored at 4° C. until further use.

Immunohistochemistry (IHC): IHC was used to stain for neurons and astrocytes along with pre- and postsynaptic markers. For neurons, polyclonal chicken anti-MAP2 was used while astrocytes were stained with rabbit polyclonal anti-GFAP antibody (abcam, cat #ab5392, ab7260). Mouse monoclonal anti-bassoon antibody was used as a presynaptic marker while rabbit polyclonal anti-homer1 antibody was used as a postsynaptic marker (abcam, cat #ab82958, ab97593). For the immunostaining assay, all liquid removal steps were performed via manual pipetting and all incubation steps occurred on a shaker. PBS with 0.1% azide was removed and blocking solution consisting of 5% normal goat serum (NGS), 2% bovine serum albumin (BSA; Fisher, cat #BP1605), and 0.5% Triton X-100 (Sigma, cat #X100) in PBS was added for 30-min. After 30-min, half of the blocking solution was removed and primary antibodies made in blocking solution were added at double the desired concentration. MAP2 and GFAP were added at 1:250 for a final concentration of 1:500 while Homer and bassoon were added at 1:50 for a final concentration of 1:100. After primary antibodies were added, the spheroid plate was placed on a shaker at 37° C. overnight for MAP2 and GFAP, and for a 3-day period for homer and bassoon. After primary antibody incubation, primary antibodies were removed, and spheroids were washed with PBS+0.3% triton X-100 (PBT). Here, half of the primary antibody solution was removed from the well and exchanged with PBT three times to remove all primary antibody. Spheroids were then washed with PBT three times for 15-min each and placed on the shaker. Secondary antibodies were made in blocking solution and were as follows: goat anti-chicken Alexa Fluor 647 was used for chicken anti-MAP2, goat anti rabbit Alexa Fluor 488 was used for rabbit anti-GFAP and rabbit anti-Homer1, and goat anti-mouse Alexa Fluor 647 was used for mouse anti-bassoon (Invitrogen, cat #A32933, A32731, A32728, respectively). Secondary antibodies were made at a concentration of 1:300 for final concentrations of 1:600 since half of the PBT was removed and exchanged with secondary antibodies in blocking solution. Spheroid plates were covered in aluminum foil and placed on the shaker at 37° C. overnight. The following day, half of the secondary antibody solution was removed and PBT rinses and washes occurred the same as described above with primary antibodies.

Fluorescent in situ Hybridization (FISH): FISH was performed to validate neuronal cell type compositions in brain region specific designer spheroids modeling the VTA and PFC. To do this, a combination of 20 ZZ oligonucleotide probes bound to the target RNA were used. Target probes included Homo sapiens tyrosine hydroxylase mRNA (Hs-TH, cat #441651; GenBank Accession Number: NM_199292.2) for dopaminergic neurons, Homo sapiens solute carrier family 17 (vesicular glutamate transporter) member 7 mRNA (Hs-SLC17A7, cat #415611; GenBank Accession Number: NM_020309.3) for glutamatergic neurons, and Homo sapiens glutamate decarboxylase 1 transcript variant GAD67 mRNA (Hs-GAD1, cat #404031; GenBank Accession Number: NM_000817.2) for GABAergic neurons according to the RNAScope Multiplex Fluorescent Reagent Kit v2 user manual (Advanced Cell Diagnostics, cat #323100). Spheroids fixed in 4% PFA were used and therefore the tissue was pre-treated according to the formalin-fixed paraffin-embedded (FFPE) preparation method, though the deparaffinizing step was skipped since spheroids were not paraffin-embedded. After spheroids were incubated in hydrogen peroxide, they were washed with DI water and incubated in protease plus for 30-min at 40° C. Protease plus was washed out with DI water and the target probes for TH, vGluT, and GAD were added such that GAD1 was assigned to channel 1 (GAD1-C1), vGluT was assigned to channel 2 (SLC17A7-C2), and TH was assigned to channel 3 (TH-C3). After hybridization of the probes, pre-amplification and amplification reagents were applied according to the user guide, where AMP1 was added for 30-min at 40° C. followed by AMP2 was added for 30-min at 40° C. and AMP3 was added for 15-min at 40° C. The fluorescent Opal 520 dye was added to channel 1 containing GAD1-C1 (excitation: 494 nm, emission 525 nm; Akoya Biosciences), the Opal 570 dye was added to channel 2 containing SLC17A7-C2 (excitation: 550 nm, emission: 570 nm; Akoya Biosciences), and the Opal 690 dye was added to channel 3 containing TH-C3 (excitation: 676 nm, emission: 694 nm; Akoya Biosciences). DAPI was added in the final step to spheroids for 30-seconds, then washed with PBS. Spheroids remained in PBS until tissue clearing reagent was added. Tissue was washed in 1× wash buffer twice for 2-min each time between incubations after probe hybridization steps. Prior to probe hybridization, spheroids were washed with DI water in accordance with the manufacturer's instructions.

Tissue Clearing: After immunostaining or FISH, ScaleS4 Tissue clearing solution was added to spheroids to reduce autofluorescence during image acquisition, as previously described (Boutin et al., 2018, Hama et al., 2011). ScaleS4 was made with 40% D-sorbitol (Sigma, cat #S6021), 10% glycerol (Sigma, cat #G2289), 4M Urea (Sigma, cat #U5378), 0.2% triton X-100, 15% DMSO (Sigma, cat #D2650) in UltraPure water (Invitrogen, cat #10977-015). ScaleS4 solution was mixed via shaking at 37° C. for two days and stored at 4° C. until future use. Before the clearing solution was added, all PBT for IHC spheroids or all wash buffer for FISH spheroids was removed from the well. For spheroids stained with IHC, the nuclear stain, Hoechst 33342 Thermo, cat #62249) was added at a 1:2000 dilution in the ScaleS4 clearing solution, and 60 μL of clearing solution plus Hoechst was added to each well. For spheroids that were stained with FISH, no nuclear stain was added since DAPI was added during the assay. Spheroid plates were wrapped in foil and placed on the shaker at 37° C. overnight. The following day, the plate was sealed with parafilm and placed at 4° C. until imaging.

### Statistics and Reproducibility

Graphical Plots: For time series plots, heatmap plots, correlation matrices and radar plots, Python 3.8 was used. For principal component analysis scatter plots, TIBCO Spotfire was used, and for column graphs, GraphPad Prism 9.1 Software was used.

Calcium Activity Analysis: Calcium oscillatory peak detection data from FLIPR recordings was obtained through ScreenWorks 5.1 (Molecular Devices). Initial peak detection analysis occurred within ScreenWorks 5.1 via the PeakPro 2.0 module. Here, all parameters were set to be the same for all wells per plate. The event polarity was always set to positive and search vector length always set to 11. The baseline, trigger level, which is automatically set to 10% above the baseline, and dynamic threshold, which is the threshold for peak detection were automatically identified by the PeakPro 2.0 module. Wells were manually checked to ensure these parameters were accurately identified prior to analysis. After analysis, data from 18 peak parameters (mean peak amplitude, peak amplitude standard deviation (SD), peak count, mean peak rate, peak rate SD, peak spacing, peaking spacing SD, mean number of EAD-like peaks per well, CTD @ 50% (peak width at 50% amplitude), CTD @ 90% (peak width at 90% amplitude), rise slope, rise slope SD, mean peak rise time, peak rise time SD, decay slope, decay slope SD, mean peak decay time, and peak decay time SD) was exported to a STATALL file that could be converted to a Microsoft Excel spreadsheet. There, percent coefficient of variance (% CV) was calculated within each plate to measure how variable each parameter exported was. Parameters were included in future analysis if they were under the threshold cutoff of 25% CV, which included peak count, peak rate, peak spacing, peak width 50% and 90%, peak amplitude, peak rise time, peak decay time, rise slope, and decay slope. All data was normalized to the average of DMSO-treated control wells within each plate. Each group represented on radar plots shows the mean in comparison to DMSO vehicle controls, which should always average to 100%. Bar plots with individual values are reported as mean±SEM.

TABLE 4
Coefficients of variance calculated from 17 peak parameters
extracted from ScreenWorks' PeakPro 2.0 analysis.

| | Plate 1 | Plate 2 | Plate 3 | Plate 4 | Plate 5 | Plate 6 | Plate 7 | Plate 8 | mean |
|---|---|---|---|---|---|---|---|---|---|
| VTA-like | | | | | | | | | |
| Peak Amplitude (RFU) | 14 | 13 | 12 | 21 | 20 | 13 | 13 | 24 | 16 |
| Peak Amplitude SD | 131 | 93 | 78 | 43 | 108 | 45 | 69 | 46 | 77 |
| Peak Count | 12 | 12 | 14 | 11 | 9 | 15 | 15 | 17 | 13 |
| Peak Rate (s) | 12 | 17 | 16 | 10 | 8 | 13 | 19 | 29 | 16 |
| Peak Rate SD | 143 | 150 | 112 | 69 | 85 | 65 | 131 | 120 | 110 |
| Peak Spacing (s) | 10 | 11 | 13 | 9 | 5 | 13 | 11 | 17 | 11 |
| Peak Spacing SD | 70 | 59 | 56 | 40 | 42 | 39 | 91 | 84 | 60 |
| Peak Width 50% | 12 | 14 | 15 | 12 | 7 | 15 | 13 | 22 | 14 |
| Peak Width 90% | 10 | 11 | 13 | 10 | 6 | 13 | 7 | 25 | 12 |
| Rise Slope (RFU) | 29 | 23 | 28 | 30 | 29 | 25 | 33 | 24 | 28 |
| Rise Slope SD | 54 | 51 | 51 | 51 | 38 | 42 | 76 | 70 | 54 |
| Peak Rise Time (s) | 28 | 19 | 28 | 18 | 20 | 16 | 44 | 31 | 26 |
| Peak Rise Time SD | 118 | 84 | 109 | 79 | 46 | 59 | 101 | 133 | 91 |
| Decay Slope (RFU) | 17 | 18 | 21 | 23 | 21 | 24 | 23 | 40 | 23 |
| Decay Slope SD | 46 | 50 | 46 | 44 | 53 | 42 | 44 | 62 | 48 |
| Peak Decay Time (s) | 12 | 16 | 16 | 11 | 4 | 26 | 29 | 58 | 22 |
| Peak Decay Time SD | 47 | 99 | 49 | 42 | 35 | 54 | 34 | 128 | 61 |
| PFC-like | | | | | | | | | |
| Peak Amplitude (RFU) | 16 | 18 | 17 | 35 | 25 | 10 | 19 | 17 | 20 |
| Peak Amplitude SD | 59 | 51 | 30 | 35 | 30 | 17 | 41 | 47 | 38 |
| Peak Count | 11 | 24 | 10 | 20 | 22 | 15 | 19 | 29 | 19 |
| Peak Rate (s) | 12 | 21 | 10 | 19 | 23 | 15 | 19 | 29 | 18 |
| Peak Rate SD | 40 | 31 | 18 | 43 | 42 | 19 | 25 | 49 | 33 |
| Peak Spacing (s) | 12 | 35 | 11 | 29 | 21 | 16 | 19 | 41 | 23 |
| Peak Spacing SD | 34 | 94 | 25 | 51 | 23 | 40 | 45 | 69 | 48 |
| Peak Width 50% | 17 | 37 | 10 | 38 | 16 | 15 | 14 | 40 | 23 |
| Peak Width 90% | 12 | 28 | 8 | 31 | 9 | 14 | 12 | 27 | 18 |
| Rise Slope (RFU) | 24 | 26 | 15 | 55 | 26 | 12 | 21 | 20 | 25 |
| Rise Slope SD | 21 | 27 | 17 | 37 | 28 | 14 | 35 | 33 | 26 |
| Peak Rise Time (s) | 43 | 39 | 7 | 70 | 39 | 11 | 13 | 19 | 30 |
| Peak Rise Time SD | 154 | 174 | 9 | 115 | 166 | 24 | 15 | 119 | 97 |
| Decay Slope (RFU) | 15 | 20 | 14 | 39 | 21 | 12 | 12 | 16 | 19 |
| Decay Slope SD | 47 | 42 | 14 | 37 | 41 | 12 | 25 | 61 | 35 |
| Peak Decay Time (s) | 11 | 32 | 8 | 18 | 8 | 13 | 7 | 20 | 14 |
| Peak Decay Time SD | 97 | 97 | 22 | 75 | 78 | 31 | 28 | 85 | 64 |

Table 4 shows percent coefficients of variance (% CV) values for 17 peak parameters obtained from peak analysis on calcium activity obtained on the FLIPR. % CV values were calculated by dividing the mean by the standard deviation then multiplying by 100 ((standard deviation/mean)*100) in Wt spheroids with no previous experimental manipulation. % CV values<30% indicated a peak parameter with low variability and those were used for future analysis and plotting

For peak detection data obtained from the Phenix Plus confocal microscope, image sequences were stored in and exported from Columbus Image Data Storage and Analysis as single plane TIFFs. Each recording was imported into ImageJ and converted to a single stack. Prior to peak detection analysis, the T-function, F div F0, in ImageJ was used to obtain calcium signals normalized to background fluorescence. The ImageJ Plugin, LC_Pro, which was first described by Francis et al., 2014, was used to automatically identify regions of interest containing dynamic calcium signals across the image sequence. The automated analysis was used on the F div F0 recording so that calcium measurements would be reported as normalized fluorescent values (F/F0). For the LC_Pro analysis, default settings were used. F/F0 values for a region of interest (ROI) were exported if they contained a high signal to noise ratio and exported to a text file titled ROI Normalized. Once this text file was converted to a csv file, it was uploaded into Python for peak detection analysis. While code for this analysis is publicly available on GitHub, a brief description of the process is described. First, data from all identified ROIs was normalized such that the minimum F/F0 value was equal to 1. Time series plots showing mean signal plus variability represented as 95% confidence interval were generated, along with heatmaps showing activity across every identified ROI, and a correlation matrix plotted as a heatmap representing correlation coefficients across all ROIs. The correlation matrix was used to describe how synchronous the calcium activity within a spheroid was, and a synchrony score was measured by calculating the average correlation coefficient across the matrix. Given that LC_Pro identifies different numbers of ROIs for each recording, the inventors used a random sample generator to randomly choose 12 ROIs for peak detection analysis. To ensure this random sample reflected the population activity, the inventors required the correlation score of the random sample to be within 5% of the correlation score of the population of ROIs. The find_peaks package was imported from scipy.signal and used for peak detection analysis. The scipy package, find_peaks was used to detect and measure peak parameters including peak count, amplitude, and width. Data was exported and peak count, amplitude, and width are reported from this detection analysis.

Statistical Analysis: Python, R Studio and GraphPad Prism were used for statistical analysis

Data used in the current study spanned eight 384-well plates, and each experiment consists of n=2-3 biological replicates with n>3 technical replicates per batch.

### Results

### Designer Neural Spheroids Exhibit Differential Calcium Activity Profiles Depending on Neuronal Subtype Composition

The inventors sought to establish whether iPSC-derived, differentiated neurons could be incorporated into a co-culture spheroid system, maintained, and have functional activity. The inventors mixed excitatory glutamatergic neurons, inhibitory GABAergic neurons, and dopamine-releasing dopaminergic neurons with astrocytes and seeded as cell mixtures of controlled ratios into 384-well, ultra-low attachment, round bottom plates to force cell aggregation into the formation of spheroids. The inventors observed spheroid formation after 3 days in culture in both PFC-like spheroids (70% glutamatergic 30% GABAergic neurons) and VTA-like spheroids (65% dopaminergic 5% glutamatergic 30% GABAergic neurons), which both consisted of 90% neurons and 10% astrocytes (data not shown). The spheroids were matured for 21 days until calcium signals were detected using a calcium fluorescence (Cal6) dye. Spheroids formed by this protocol were ˜300-350 mm in diameter after the maturation process and had a homogenous spatial distribution of neurons and astrocytes (MAP and GFAP staining) and lacked a necrotic core (nuclear staining). The mature functional spheroids also expressed pre- and postsynaptic markers as shown by synapsin and homer staining distributed evenly throughout spheroids, supporting the presence of synaptic connections.

To test whether this spheroid system is compatible with high-throughput (HT) study designs, the inventors measured calcium activity across all wells per plate simultaneously using a whole plate reader equipped with a high speed, high sensitivity EMCCD camera for both fluorescent and luminescent detection (the FLIPR Penta High-Throughput Cellular Screening System). The inventors analyzed the measured calcium oscillations in spheroids incubating in a calcium 6 (Cal6) dye for high reproducibility peak parameters using ScreenWorks PeakPro 2.0 analysis. Specifically, the inventors analyzed 17 peak parameters and selected ten reproducible parameters with low variability (<30% coefficient of variance (% CV; Table 4). The inventors performed an initial proof-of-concept study measuring calcium activity in 16 different spheroid types that all contained 90% neurons and 10% astrocytes but differed in their neuronal subtype composition to assess whether changing neuronal cell type composition would impact phenotypic profiles. Principal component analysis (PCA) was used to analyze the multidimensional peak data and scatter plots were produced showing datapoints from individual spheroids when plotted against the first two components of the PCA (FIG. 21A, 21B). The inventors observed that single neuron spheroids (SNSs, 90% neuron 10% astrocyte) consisting of only dopaminergic, glutamatergic, or GABAergic neurons form distinct clusters, suggesting unique phenotypic profiles, and that spheroids with controlled gradient ratios of multiple neuronal cell types cluster near the SNS cluster with the same dominant neuronal cell type (FIG. 21B). Together, this data shows that altering neuronal cell type composition within spheroids produces unique calcium activity phenotypes.

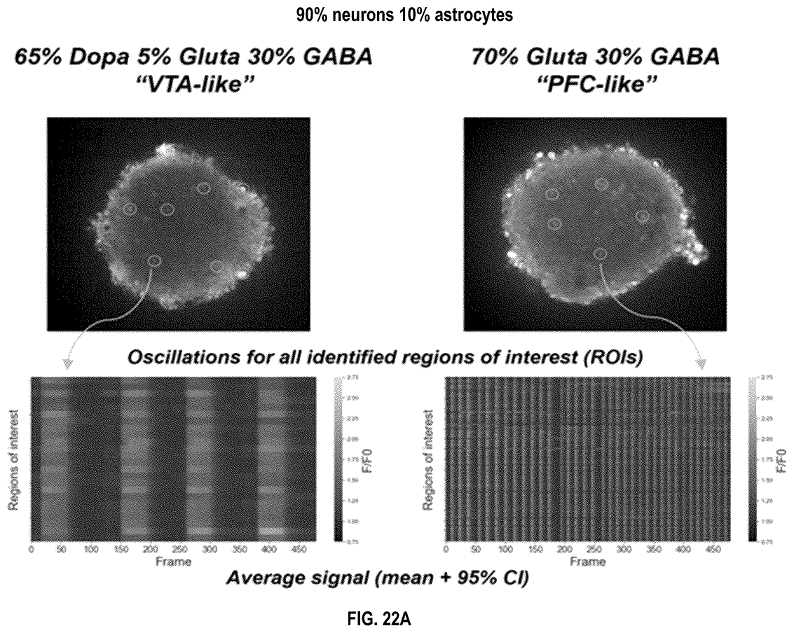

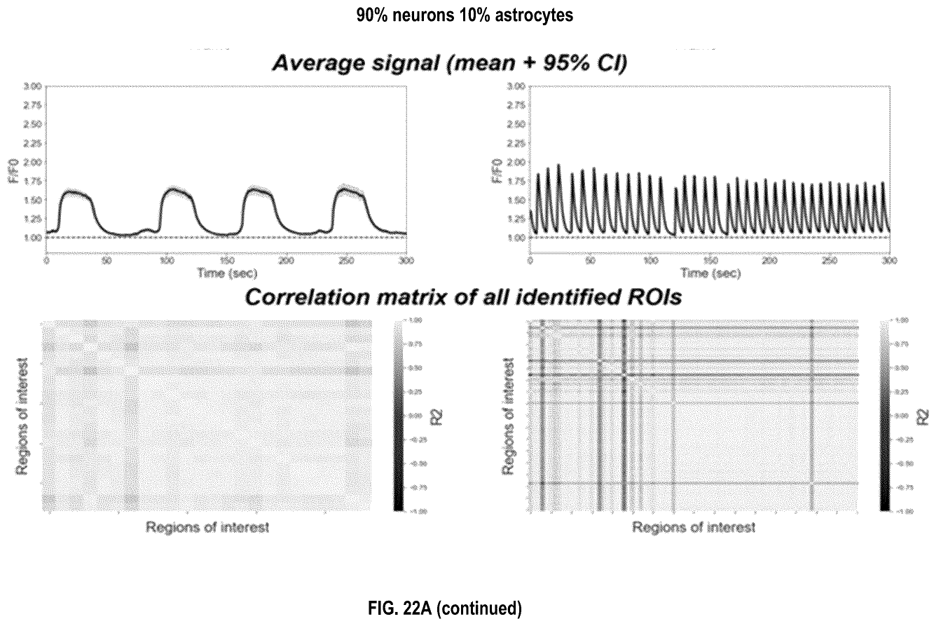

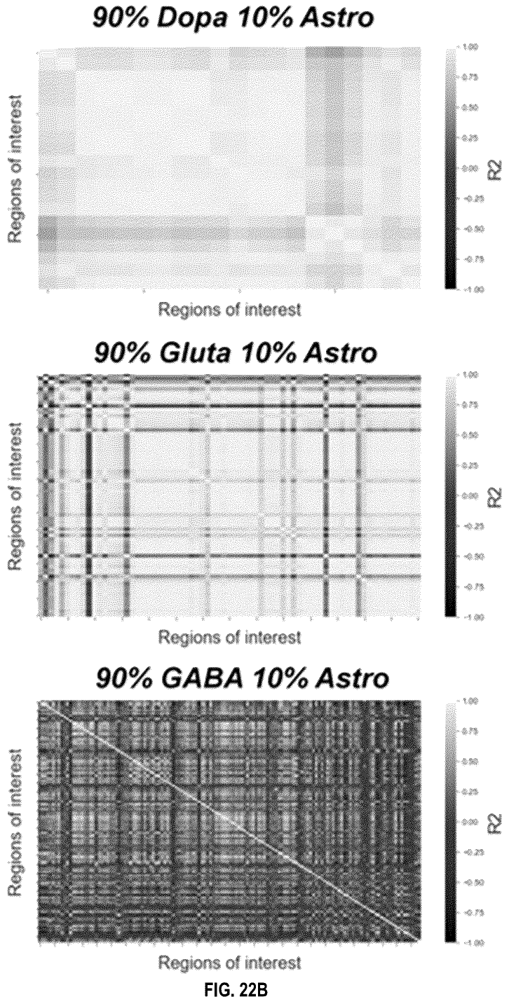

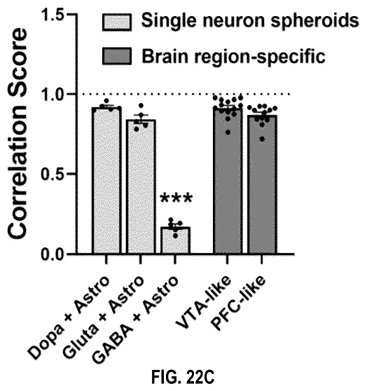

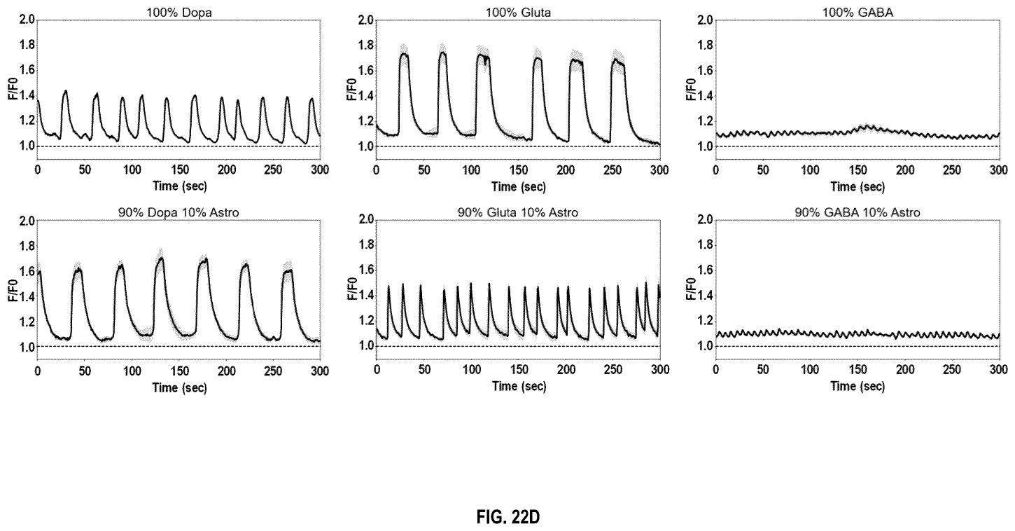

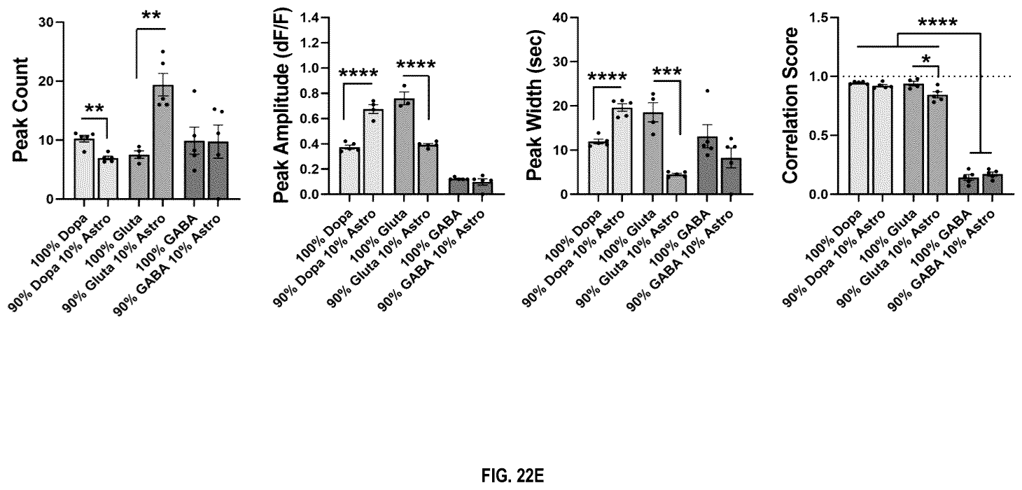

After establishing culture maturation conditions and maintenance of individual and heterogenous neuronal subtypes, the inventors created spheroids with controlled cell compositions mimicking the human prefrontal cortex or the ventral tegmental area (noted here as PFC-like spheroids or VTA-like spheroids, respectively). Although both brain region-specific neural spheroids consisted of 90% neurons and 10% astrocytes, VTA-like spheroids were created by combining 65% Dopa, 5% Gluta, and 30% GABA neurons while the PFC-like spheroids were created with 70% Gluta and 30% GABA neurons, based on previous reports quantifying neuronal cell type distributions in human brains (Lin et al., 2013; Pignatelli et al., 2015; Root et al., 2016). Next the inventors measured calcium activity from individual cells within single neuron spheroids (SNSs) and brain region-specific spheroids incubated in Cal6 dye using a Phenix Plus automated confocal microscope. Regions of interest (ROIs) with oscillatory patterns were automatically identified using the LC_Pro plugin through ImageJ and activity of all identified ROIs was plotted as a heatmap (FIG. 22A). Population activity was represented by the mean plus 95% confidence interval of all identified ROIs (FIG. 22B, time series trace). In contrast to the FLIPR assay that measures population activity within a spheroid, synchrony could be measured with this assay since the activity of individual cells within the spheroid was obtained and therefore, correlation matrices indicating correlation coefficient (R²) between all identified cells (ROIs) were plotted as a heatmap, and the average R² value from each spheroid was calculated as its “correlation score” (FIGS. 22A, 22B). Here the inventors surprising found that that the VTA- and PFC-like spheroids display unique phenotypic profiles that are similar to the phenotypes displayed by the SNSs with dominant neuronal cell types (i.e., SNSs with dopaminergic or glutamatergic neurons, respectively; FIGS. 22A, 22D). Furthermore, astrocytes were not necessary for neuronal activity in spheroids, but their presence was found to alter phenotypes in SNSs containing dopaminergic or glutamatergic neurons (FIG. 22D). Given that astrocytes can alter specific peak parameters such as peak width, which may be an indicator of neurotransmitter release, along with the role they play in synaptic plasticity and neurological diseases, spheroids tested throughout the rest of the study were all made with 90% neurons and 10% astrocytes and only differed by neuronal subtype composition. In addition, synchronous activity was observed in SNSs with dopaminergic and glutamatergic neurons along with both brain region-specific spheroid types, but not in SNSs with GABAergic neurons (FIGS. 22B, 22C). Together, these data indicate that brain region-specific spheroids display unique phenotypic profiles and that changes in synchronous neuronal activity in these spheroids occurs through GABAergic mechanisms.

Phenotypic Profiles in Brain Region-Specific Spheroids can be Differentially Modulated with Compounds Targeting Neuronal Subtype Receptors

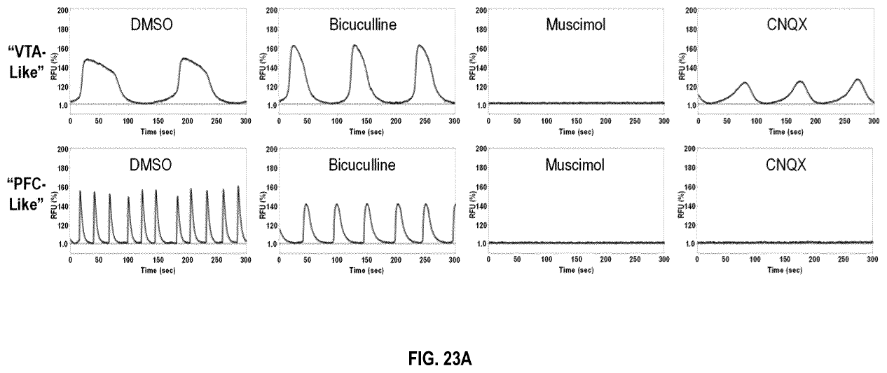

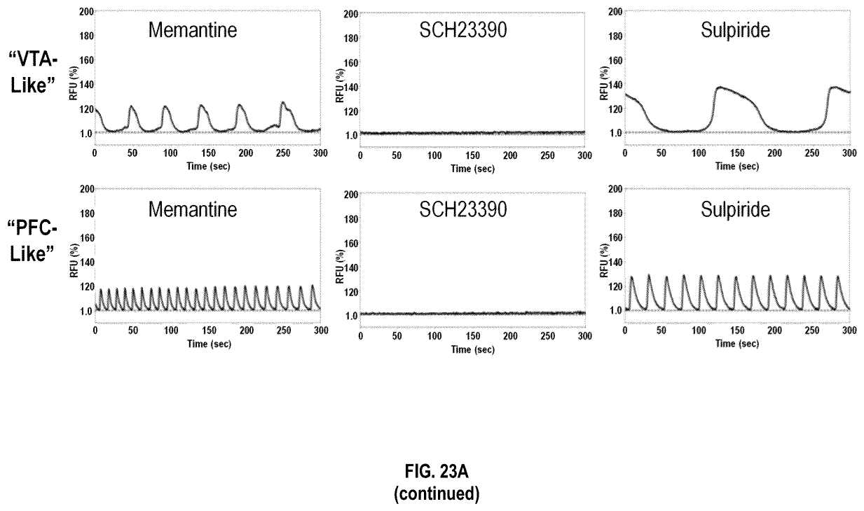

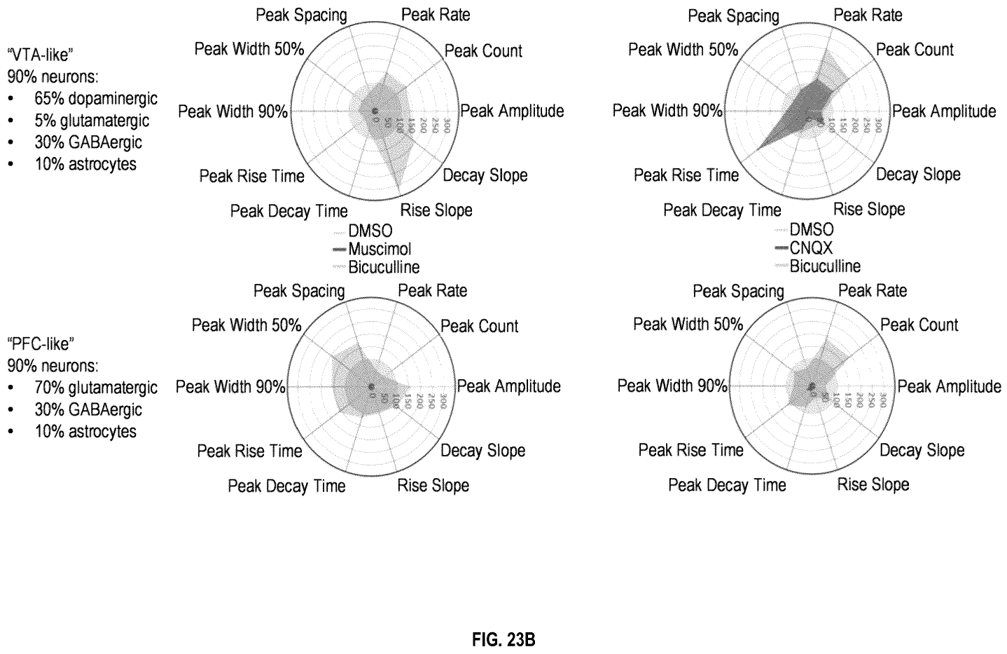

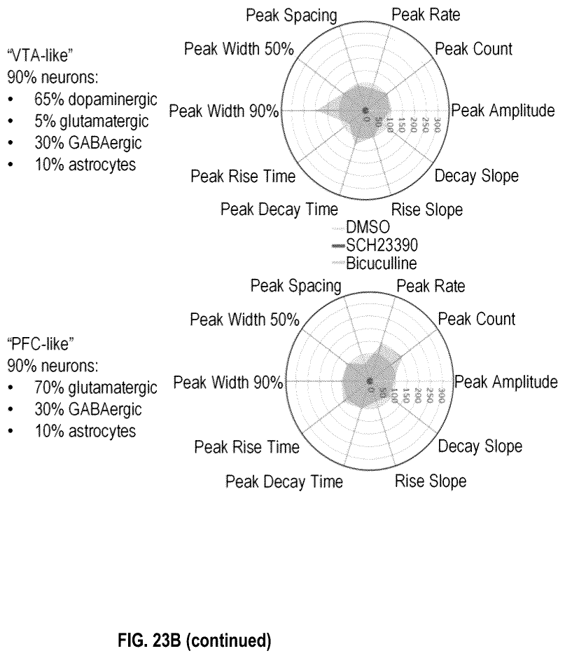

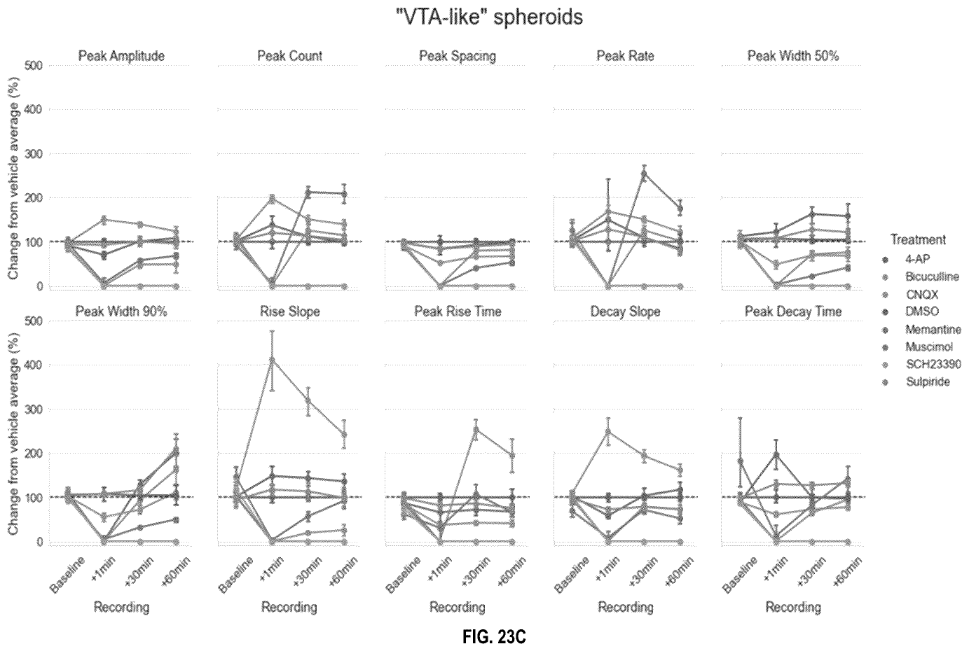

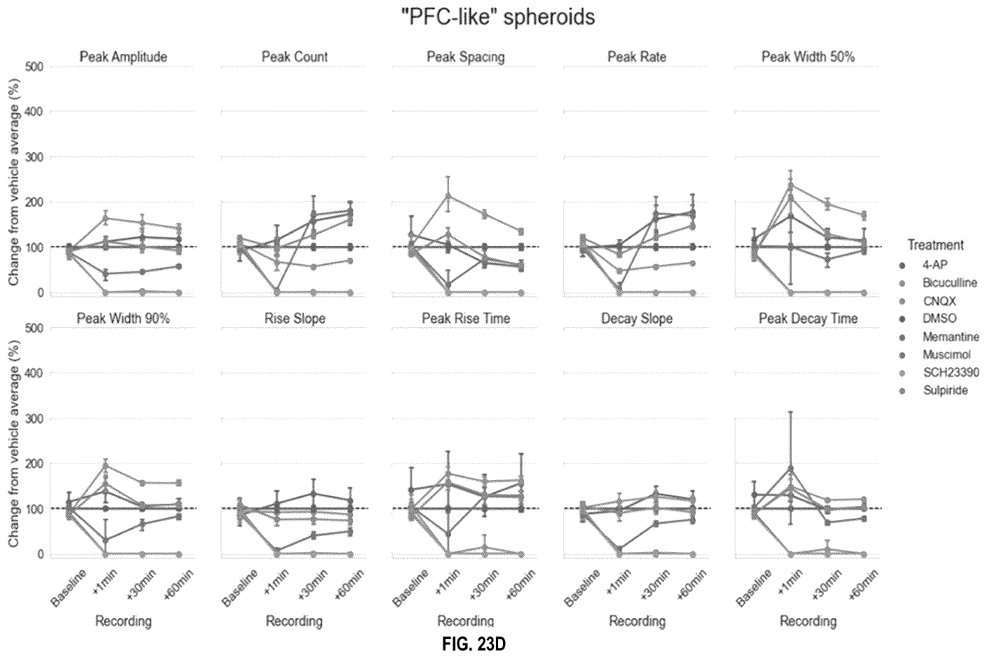

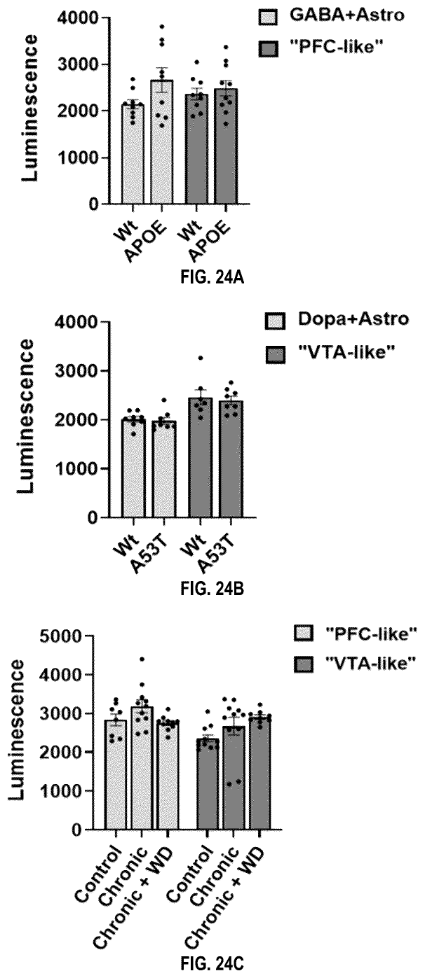

The inventors validated neuronal spheroid functional response via treatment with compounds of known mechanism, termed here as quality control (QC) compounds, that targeted receptors on each neuronal subtype (FIGS. 23A, 23B, 23C, 23D, Table 5). In both spheroid types, treatment with the GABA_{A} receptor (GABA_{A}R) agonist muscimol led to a complete inhibition of activity that began immediately after treatment and persisted for at least 60-min (FIGS. 23A, 23B, 23C, 23D, Table 5). Conversely, treatment with GABA_{A}R antagonist, bicuculline, induced spheroid type-dependent changes; in VTA-like spheroids, enhanced activity was apparent through increases in peak count, rate, and amplitude with decreases in peak rise and decay time while in PFC-like spheroids, peak count was reduced while peak amplitude and peak width was increased, suggesting different functional responses between the spheroids to GABA_{A}R antagonism (FIGS. 23A-23D, Table 5). To target Gluta neurons, spheroids were treated with the α-amino-hydroxy-5-methyl-4-isoxazolepropionic acid receptor (AMPAR) antagonist, CNQX, and the N-methyl-D-aspartate receptor (NMDAR) antagonist, memantine. Memantine similarly affected the two spheroid types, with effects including increases in peak count and rate and decreases in peak amplitude (FIGS. 23A-23D, Table 5). CNQX inhibited all activity in PFC-like spheroids for all three recordings (1-, 30-, and 60-min), but this total inhibition was only observed in VTA-like spheroids 1-min after treatment, with activity rebounding by the 30-min recording, indicated by increases in peak rise time and peak count (FIGS. 23A-23D, Table 5). These data suggest that the differences in number of Gluta neurons between the two spheroid types (70% in PFC-like and 5% in VTA-like spheroids) changes the threshold for response to compounds targeting these receptors, presumably because of lower AMPAR expression in VTA-like compared to PFC-like spheroids. SCH23390 was used to block dopamine 1/5 receptors (D1 Rs), which are stimulatory G-protein coupled receptors, while sulpiride was used to block dopamine 2/3 receptors (D2Rs), which are inhibitory G-protein coupled receptors. In both spheroid types, D1R antagonism with CNQX led to total inhibition that lasted throughout the 1-hr period (FIGS. 23A-23D, Table 5). In PFC-like spheroids, D2R antagonism with sulpiride increased peak count and rate while decreasing peak spacing. However, in VTA-like spheroids, no changes were observed in measures of peak frequency but increases in peak width and peak decay time were observed (FIGS. 23A-23D, Table 5). Together, this data shows that predictable functional responses occurred in brain region-specific spheroids based on their neuronal cell type composition. Further, calcium activity of brain region-specific spheroids can be differentially modulated with control compounds targeting cell type specific receptors based on their neuronal cell type composition.

TABLE 5
Statistical analysis of functional responses to control compounds with 2-way repeated measures ANOVA.

| Multiple | | | | | | | | Rise | Decay | | |
|---|---|---|---|---|---|---|---|---|---|---|---|
| Comparisons | Recording | PkA | PkCt | PkRate | PkSp | PkW50 | PkW90 | Slope | Slope | PkRt | PkDt |

VTA-like spheroids

| 4-AP - DMSO | Baseline | | 0.374 | 1.000 | 1.000 | 0.985 | <0.0001 | 0. [symbol] | 1.000 | 1.000 | 0.741 | 0.8 [symbol] |
|---|---|---|---|---|---|---|---|---|---|---|---|---|
| Bicuculline - DMSO | Baseline | | 0.945 | 1.000 | 0.9 [symbol] 8 | 0.284 | 0. [symbol] | 1.000 | 0.8 [symbol] | 0.9 [symbol] | 0.581 | 0. [symbol] 40 |
| CNQX - DMSO | Baseline | | 0.070 | 0. [symbol] | 0.371 | 0.041 | 1.000 | 1.000 | 1.000 | 1.000 | 0.998 | 0. [symbol] 47 |
| Memantine - DMSO | Baseline | | 0.997 | 0.91 [symbol] | 0. [symbol] | 0.722 | 1.000 | 1.000 | 0.024 | 0.002 | 0.00 [symbol] | <0.0001 |
| Muscimol - DMSO | Baseline | | 0.12 [symbol] | 0.9 [symbol] | 0.9 [symbol] | 0.481 | 0.9 [symbol] | 1.000 | 1.000 | 0. [symbol] | 0. [symbol] 74 | 1.000 |
| SCH23390 - DMSO | Baseline | | 0.141 | 0.992 | 1.000 | 0.735 | 0.242 | 1.000 | 0.739 | 0. [symbol] | 0.087 | 0.912 |
| Sulpiride - DMSO | Baseline | | 0.735 | 1.000 | 1.000 | 0.9 [symbol] | 1.000 | 1.000 | 0.918 | 0.8 [symbol] 3 | 1.000 | 1.000 |
| 4-AP - DMSO | +1 | min | <0.0001 | <0.0001 | 0.01 [symbol] | 0.0 [symbol] | <0.0001 | <0.0001 | 0.006 | <0.0001 | 0.002 | <0.0001 |
| Bicuculline - DMSO | +1 | min | <0.0001 | <0.0001 | <0.0001 | <0.0001 | <0.0001 | 0.001 | <0.0001 | <0.0001 | <0.0001 | 0.004 |
| CNQX - DMSO | +1 | min | <0.0001 | <0.0001 | <0.0001 | <0.0001 | <0.0001 | <0.0001 | <0.0001 | <0.0001 | <0.0001 | <0.0001 |
| Memantine - DMSO | +1 | min | <0.0001 | <0.0001 | <0.0001 | <0.0001 | <0.0001 | <0.0001 | <0.0001 | <0.0001 | <0.0001 | <0.0001 |
| Muscimol - DMSO | +1 | min | <0.0001 | <0.0001 | <0.0001 | <0.0001 | <0.0001 | <0.0001 | <0.0001 | <0.0001 | <0.0001 | <0.0001 |
| SCH23390 - DMSO | +1 | min | <0.0001 | <0.0001 | <0.0001 | <0.0001 | <0.0001 | <0.0001 | <0.0001 | <0.0001 | <0.0001 | <0.0001 |
| Sulpiride - DMSO | +1 | min | 0. [symbol] | 0.284 | 0. [symbol] | 0.013 | 0.922 | 0. [symbol] | 0. [symbol] | 0.003 | 0.307 | 0.1 [symbol] 4 |
| 4-AP - DMSO | +30 | min | 1.000 | 0.389 | 0.9 [symbol] | 0. [symbol] | <0.0001 | 0.1 [symbol] | 0.004 | 1.000 | 0.014 | 1.000 |
| Bicuculline - DMSO | +30 | min | <0.0001 | <0.0001 | 0.002 | <0.0001 | 0.010 | 0.016 | <0.0001 | <0.0001 | <0.0001 | 0.0 [symbol] 1 |
| CNQX - DMSO | +30 | min | <0.0001 | 0.002 | 0. [symbol] 73 | <0.0001 | 0.031 | 0.9 [symbol] | <0.0001 | 0.042 | <0.0001 | 0.015 |
| Memantine - DMSO | +30 | min | <0.0001 | <0.0001 | <0.0001 | <0.0001 | <0.0001 | <0.0001 | 0.020 | 0.008 | 0. [symbol] | 0. [symbol] 9 |
| Muscimol - DMSO | +30 | min | <0.0001 | <0.0001 | <0.0001 | <0.0001 | <0.0001 | <0.0001 | <0.0001 | <0.0001 | <0.0001 | <0.0001 |
| SCH23390 - DMSO | +30 | min | <0.0001 | <0.0001 | <0.0001 | <0.0001 | <0.0001 | <0.0001 | <0.0001 | <0.0001 | <0.0001 | <0.0001 |
| Sulpiride - DMSO | +30 | min | 1.000 | 0.217 | 0.9 [symbol] | 0.020 | 0.021 | 0. [symbol] 85 | 0.997 | 0.006 | 0. [symbol] | 0.079 |
| 4-AP - DMSO | + [symbol] 0 | min | 0. [symbol] | 1.000 | 0. [symbol] | 1.000 | <0.0001 | <0.0001 | 0.03 [symbol] | 0.232 | 0.002 | 1.000 |
| Bicuculline - DMSO | + [symbol] 0 | min | <0.0001 | <0.0001 | 0.495 | <0.0001 | 0.007 | 0.992 | <0.0001 | <0.0001 | <0.0001 | 0.244 |
| CNQX - DMSO | + [symbol] 0 | min | <0.0001 | 0.24 [symbol] | 1.000 | <0.0001 | 0.116 | <0.0001 | <0.0001 | 0.003 | <0.0001 | 0. [symbol] |
| Memantine - DMSO | + [symbol] 0 | min | <0.0001 | <0.0001 | <0.0001 | <0.0001 | <0.0001 | <0.0001 | 0. [symbol] | <0.0001 | 0.01 [symbol] | 0.009 |
| Muscimol - DMSO | + [symbol] 0 | min | <0.0001 | <0.0001 | <0.0001 | <0.0001 | <0.0001 | <0.0001 | <0.0001 | <0.0001 | <0.0001 | <0.0001 |
| SCH23390 - DMSO | + [symbol] 0 | min | <0.0001 | <0.0001 | <0.0001 | <0.0001 | <0.0001 | <0.0001 | <0.0001 | <0.0001 | <0.0001 | <0.0001 |
| Sulpiride - DMSO | + [symbol] 0 | min | 0.978 | 0.9 [symbol] 1 | 0. [symbol] | 0.423 | 0.281 | <0.0001 | 0. [symbol] | <0.0001 | 0.077 | 0.023 |

PFC-like spheroids

| 4-AP - DMSO | Baseline | | 0.755 | 1.000 | 1.000 | 0.230 | 1.000 | <0.0001 | 0.999 | 0. [symbol] 7 | 0. [symbol] 31 | 0.31 [symbol] |
|---|---|---|---|---|---|---|---|---|---|---|---|---|
| Bicuculline - DMSO | Baseline | | 0.4 [symbol] | 0.850 | 0.813 | 0.983 | 0.011 | 0.015 | 0.998 | 0.992 | 0. [symbol] 75 | 0.7 [symbol] 0 |
| CNQX - DMSO | Baseline | | 0.186 | 0.998 | 0. [symbol] | 1.000 | 0.111 | 0.144 | 0.949 | 0. [symbol] | 1.000 | 0. [symbol] 4 |
| Memantine - DMSO | Baseline | | 0.280 | 0. [symbol] | 1.000 | 0.999 | 0. [symbol] 2 | 1. [symbol] 00 | 0.991 | 0.581 | 0.917 | 1.000 |
| Muscimol - DMSO | Baseline | | 0.203 | 0. [symbol] 1 | 0. [symbol] | 0. [symbol] 1 | 0.1 [symbol] 2 | 0.172 | 0.993 | 1.000 | 0.4 [symbol] 0 | 0. [symbol] 1 |
| SCH23390 - DMSO | Baseline | | 0.069 | 0.484 | 0.422 | 0.378 | 0.02 [symbol] | 0.009 | 0.909 | 0.999 | 0.041 | 0. [symbol] |
| Sulpiride - DMSO | Baseline | | 0.863 | 0.1 [symbol] | 0.210 | 0.13 [symbol] | 0.020 | 0.006 | <0.0001 | 0.451 | <0.0001 | 0.84 [symbol] |
| 4-AP - DMSO | +1 | min | 0.174 | 0. [symbol] 11 | 1.000 | 1.000 | <0.0001 | <0.0001 | <0.0001 | 1.000 | 1.000 | 0.349 |
| Bicuculline - DMSO | +1 | min | <0.0001 | 0.075 | <0.0001 | <0.0001 | <0.0001 | <0.0001 | 1.000 | 0.05 [symbol] | 0.001 | 0.010 |
| CNQX - DMSO | +1 | min | <0.0001 | <0.0001 | <0.0001 | <0.0001 | <0.0001 | <0.0001 | <0.0001 | <0.0001 | <0.0001 | <0.0001 |
| Memantine - DMSO | +1 | min | <0.0001 | <0.0001 | <0.0001 | <0.0001 | <0.0001 | <0.0001 | <0.0001 | <0.0001 | <0.0001 | 0.007 |
| Muscimol - DMSO | +1 | min | <0.0001 | <0.0001 | <0.0001 | <0.0001 | <0.0001 | <0.0001 | <0.0001 | <0.0001 | <0.0001 | <0.0001 |
| SCH23390 - DMSO | +1 | min | <0.0001 | <0.0001 | <0.0001 | <0.0001 | <0.0001 | <0.0001 | <0.0001 | <0.0001 | <0.0001 | <0.0001 |
| Sulpiride - DMSO | +1 | min | 0.050 | 1.000 | 0.374 | 0.076 | <0.0001 | <0.0001 | 0. [symbol] 3 | 0. [symbol] | 0.003 | 0.024 |
| 4-AP - DMSO | +30 | min | <0.0001 | <0.0001 | <0.0001 | <0.0001 | 0. [symbol] | 0.999 | <0.0001 | <0.0001 | 0.011 | 1.000 |
| Bicuculline - DMSO | +30 | min | <0.0001 | 0.002 | <0.0001 | <0.0001 | <0.0001 | <0.0001 | 0. [symbol] | 0.001 | 0.077 | 0.72 [symbol] |
| CNQX - DMSO | +30 | min | <0.0001 | <0.0001 | <0.0001 | <0.0001 | <0.0001 | <0.0001 | <0.0001 | <0.0001 | <0.0001 | <0.0001 |
| Memantine - DMSO | +30 | min | <0.0001 | <0.0001 | <0.0001 | 0.002 | 0.003 | <0.0001 | <0.0001 | <0.0001 | 0.9 [symbol] | 0.016 |
| Muscimol - DMSO | +30 | min | <0.0001 | <0.0001 | <0.0001 | <0.0001 | <0.0001 | <0.0001 | <0.0001 | <0.0001 | <0.0001 | <0.0001 |
| SCH23390 - DMSO | +30 | min | <0.0001 | <0.0001 | <0.0001 | <0.0001 | <0.0001 | <0.0001 | <0.0001 | <0.0001 | <0.0001 | <0.0001 |
| Sulpiride - DMSO | +30 | min | 0. [symbol] 7 | 0.110 | 0.242 | 0.0 [symbol] 2 | 0.10 [symbol] | 1.000 | 1.000 | 0. [symbol] 73 | 0. [symbol] | 1.000 |
| 4-AP - DMSO | + [symbol] 0 | min | 0.001 | <0.0001 | <0.0001 | <0.0001 | 0. [symbol] 7 | 0. [symbol] | <0.0001 | <0.0001 | 1.000 | 1.000 |
| Bicuculline - DMSO | + [symbol] 0 | min | <0.0001 | 0.101 | 0.00 [symbol] | 0.011 | <0.0001 | <0.0001 | 1.000 | 0.013 | 0.032 | 0. [symbol] 52 |
| CNQX - DMSO | + [symbol] 0 | min | <0.0001 | <0.0001 | <0.0001 | <0.0001 | <0.0001 | <0.0001 | <0.0001 | <0.0001 | <0.0001 | <0.0001 |
| Memantine - DMSO | + [symbol] 0 | min | <0.0001 | <0.0001 | <0.0001 | <0.0001 | 0. [symbol] 7 | 0.009 | <0.0001 | 0.002 | 0. [symbol] | 0.1 [symbol] 2 |
| Muscimol - DMSO | + [symbol] 0 | min | <0.0001 | <0.0001 | <0.0001 | <0.0001 | <0.0001 | <0.0001 | <0.0001 | <0.0001 | <0.0001 | <0.0001 |
| SCH23390 - DMSO | + [symbol] 0 | min | <0.0001 | <0.0001 | <0.0001 | <0.0001 | <0.0001 | <0.0001 | <0.0001 | <0.0001 | <0.0001 | <0.0001 |
| Sulpiride - DMSO | + [symbol] 0 | min | 1.000 | <0.0001 | <0.0001 | <0.0001 | 0. [symbol] 94 | 0. [symbol] 4 | 0.0 [symbol] | 0. [symbol] | 0. [symbol] | 1.000 |

[symbol]

indicates data missing or illegible when filed

Table representing p-values from multiple comparisons analysis with Sidak's post hoc test comparing responses from each control compound to DMSO controls for each of the 10 peak parameters analyzed. Data was analyzed using linear mixed model ANOVA where treatment was a between-subjects factor and recording was a within-subjects factor to examine repeated measures. Significant treatment×recording interactions (p<0.05) were followed up with Sidak's post hoc test, and p-values are displayed on the table, with those as bold being significantly different from DMSO.

Incorporating Genetically Engineered GABA Neurons Expressing APOE e4/4 Allele Produces a Predictive Calcium Activity Phenotype that is Reversed with Clinically Approved Treatments for Alzheimer's Disease

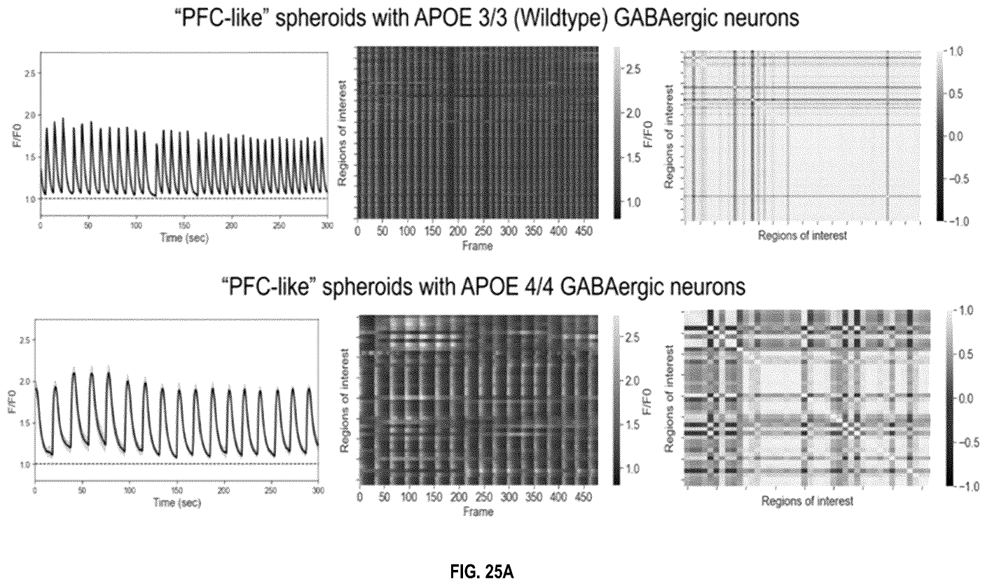

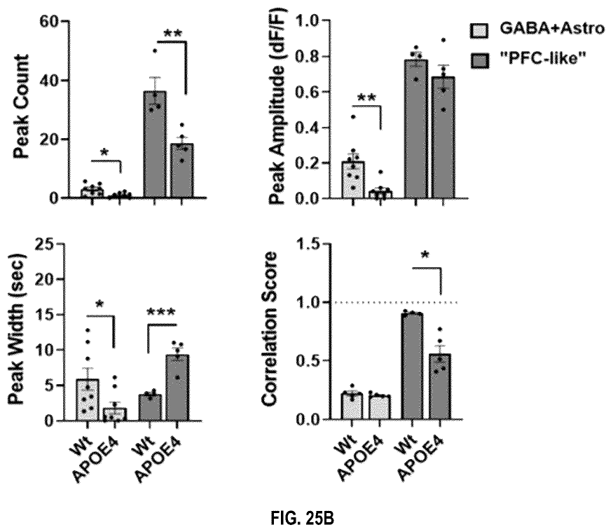

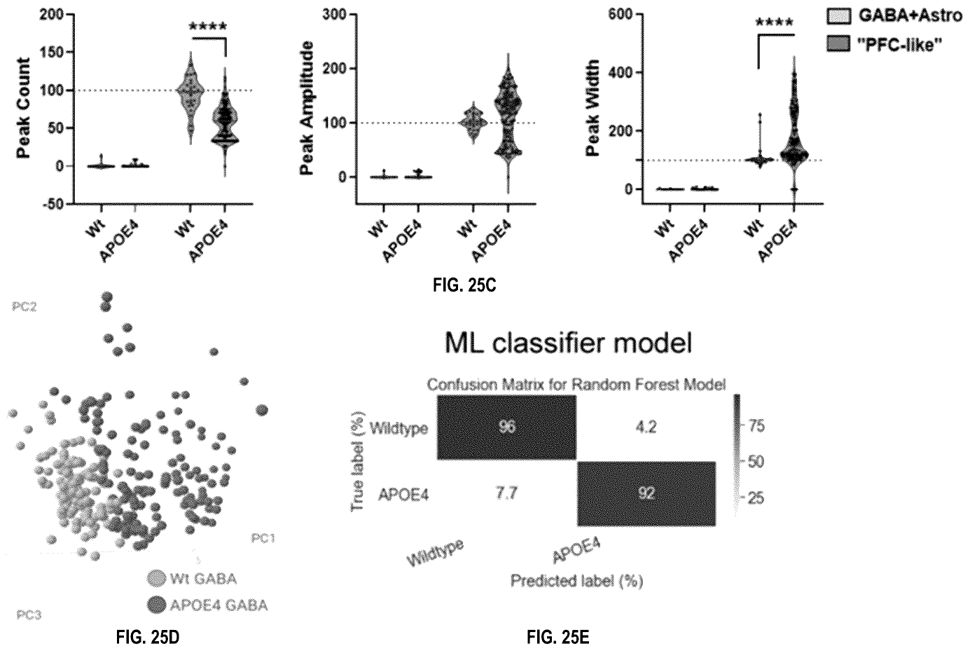

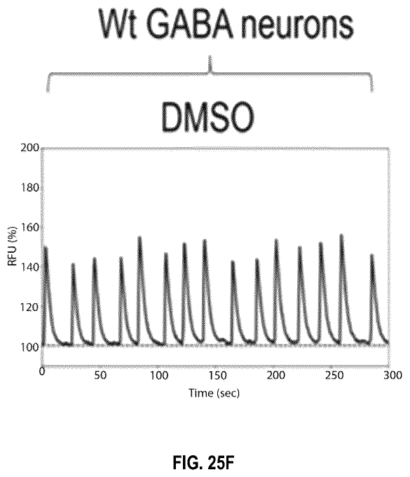

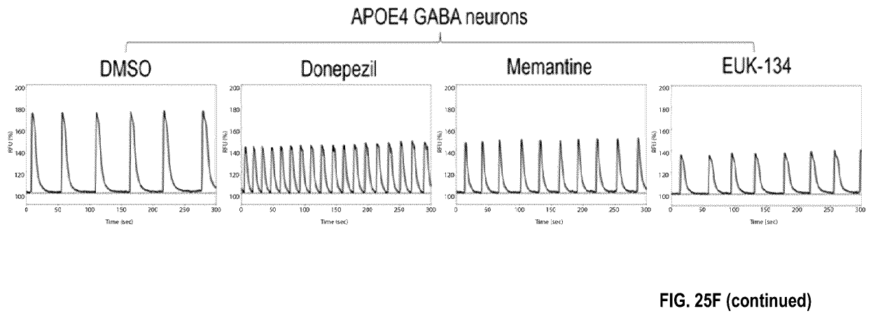

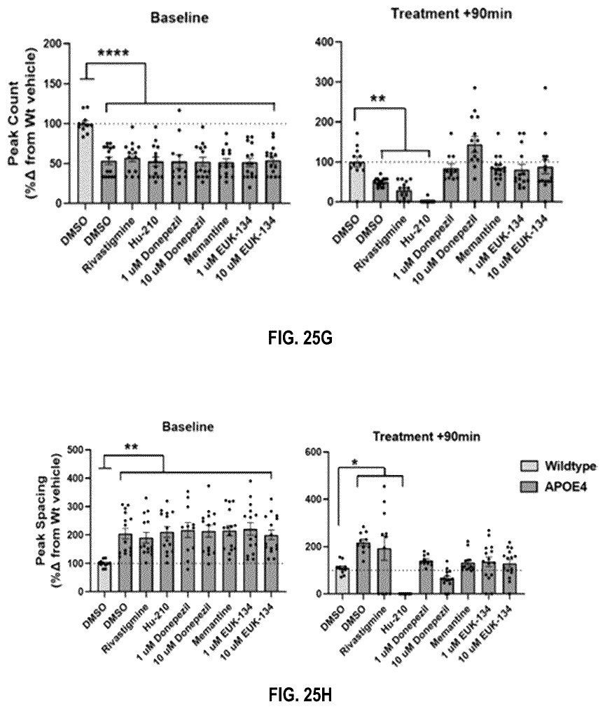

To model Alzheimer's Disease (AD), GABA neurons that were genetically engineered to carry the apolipoprotein e4/4 (APOE4) allele, a genotype associated with AD, were incorporated into spheroids on the day they were generated. The APOE4 GABA neurons were engineered from the same donor's cell line as the wildtype (Wt) GABA neurons, which express the APOE e3/3 (APOE3) allele, a genotype with no association for developing AD (Belloy et al., 2019). PFC-like spheroids were made either containing 30% APOE3 (Wt) GABA neurons or 30% APOE4 (mutant) GABA neurons, and single neuron GABA spheroids with either Wt or APOE4 GABA neurons were made as controls. After a 3-week maintenance period, spheroid viability was measured with a 3D Cell Titer Glo (CTG) assay kit and no significant differences were observed between APOE3 and APOE4 GABA neurons both for single neuron GABA spheroids or PFC-like spheroids, suggesting that functional differences observed between genotypes would not attributed to cell viability differences caused by the APOE4 mutation (FIGS. 25A, 25B, 25C).

To assess functional differences between genotypes, spheroids were incubated in Cal6 dye, baseline calcium activity was recorded using a PhenixPlus automated confocal microscope, and peaks from individual ROIs identified with LC_Pro were quantified with the find_peaks package in Python. Peak detection analysis showed that both PFC-like and single neuron GABA spheroids with APOE4 GABA neurons displayed reduced peak count (FIGS. 25A, 25B). Additionally, peak amplitude was reduced only in SNSs with APOE4 GABA neurons, however peak width was decreased in SNSs but increased in PFC-like spheroids with APOE4 GABA neurons (FIGS. 25A, 25B). In PFC-like spheroids, the incorporation of APOE4 GABA neurons also disrupted synchronous neuronal activity, as indicated by reduced correlation scores (FIGS. 25A, 25B). Baseline functional differences between APOE genotypes in SNSs with GABAergic neurons and PFC-like spheroids were also assessed with a FLIPR prior to treating spheroids with compounds used to treat the symptoms of AD. Here, APOE4 GABA neurons in PFC-like spheroids caused a reduction in peak count and an increase in peak width, replicating the differences observed with the confocal recordings (FIGS. 25A-25I). Given the non-synchronous activity of SNSs with GABAergic neurons, the FLIPR was unable to detect peaks in these spheroids, and therefore, functional differences in genotypes in this spheroid type were not observed (FIGS. 25A-25I). To quantify how predictive the APOE4 phenotype was for the AD model, the inventors implemented a supervised machine learning algorithm to measure labeling accuracy of genotypes based on multiparametric FLIPR data in PFC-like spheroids (FIGS. 25A-25I). Principal component analysis (PCA) was used as a dimension reduction analysis prior to fitting data to a Random Forest Classifier (RFC) model, which was trained with 80% of the dataset and tested with the other 20% of the dataset, and the PCA data is plotted as a scatter plot color-coded by genotype (FIGS. 25A-25I). Prior to using the RFC model, a t-test was used to ensure no significant differences between data in the training versus the test sets, and z-tests were used to ensure similar frequencies of each genotype were represented. Here, the RFC model accurately predicted the label of 96% of Wt (APOE3) PFC-like spheroids and 92% of APOE4 PFC-like spheroids, giving an average accuracy score of 94% (FIGS. 25A-25I). Analyzing the baseline data for our AD model indicated two things: genotypic differences produced deficits in baseline calcium activity phenotypic profiles and these phenotypic profiles showed high predictive accuracy when tested against a RFC machine learning algorithm. Machine learning algorithms are superior in detecting trends and/or differences in complex systems, especially with high levels of noise, subtle presentations, and/or high volumes of data.

The inventors determined whether APOE4-induced deficits in PFC-like spheroids could be reversed following treatment with three clinically approved compounds used to treat the symptoms of AD in humans along with two preclinical compounds that are known to inhibit beta-amyloid plaques. To do this, FLIPR recordings were obtained 30-,60-, and 90-min after compound treatment in the same spheroids analyzed herein. The clinically approved compounds included cholinesterase inhibitors (Rivastigmine and Donepezil) along with an NMDAR antagonist (Memantine) while the preclinical compounds were Hu-210 and EUK-134, which have both been shown to inhibit beta-amyloid plaque production through cannabinoid receptors or inhibiting oxidative stress pathways, respectively (Bahramikia and Yazdanparast, 2013; Chen er al., 2010; Jekabsone et al., 2006; Ramirez et al., 2005). Controls included both Wt and APOE4 PFC-like spheroids that were treated with vehicle (DMSO), and all data was normalized to Wt DMSO-treated controls. First the inventors found that baseline deficits were consistent between all treatment groups prior to compound addition, and show significant reductions in peak count as well as increases in peak spacing among all treatment groups in APOE4 PFC-like spheroids compared to Wt controls (FIGS. 25A-25I). Compounds were added to wells containing spheroids via a 384-well pin tool, and treatment effects were captured 30-,60-, and 90-min after treatment. Three compounds were found to reverse deficits caused by APOE4 GABA neurons including Memantine (10 μM), Donepezil (1 & 10 uM), and EUK-134 (1 & 10 μM) since deficits in peak count and spacing were no longer significantly different from Wt DMSO-treated spheroids (FIGS. 25A-25I). In Wt PFC-like spheroids, these same compounds similarly impacted peak count and spacing (FIGS. 25A-25I). Radar plots were generated for each of these three compounds to illustrate the phenotypic profiles across all peak parameters 90 minutes (min) after treatment compared to both Wt and APOE4 PFC-like spheroids treated with DMSO (FIGS. 26A, 26B). In summary, these data show that functional deficits caused by the APOE4 mutation in GABA neurons can be reversed and that the APOE4 GABA PFC-like spheroids described herein may used in high-throughput drug screening studies.

A Mutant A53T SNCA Model of Parkinson's Disease Produces Predictive Phenotypic Deficits in Calcium Activity that can be Reversed with a Dopamine Agonist

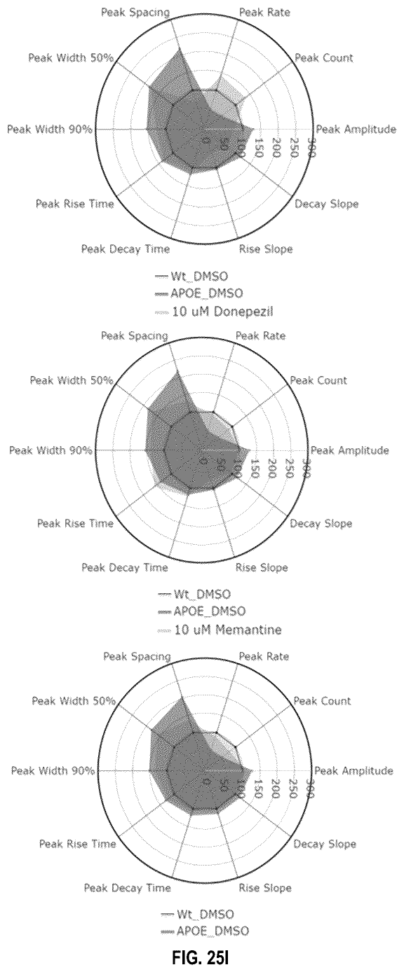

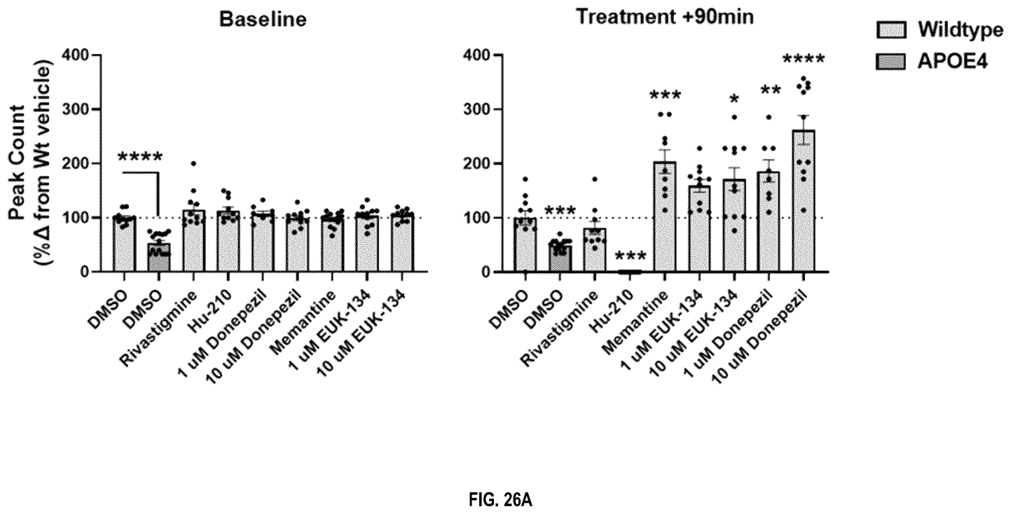

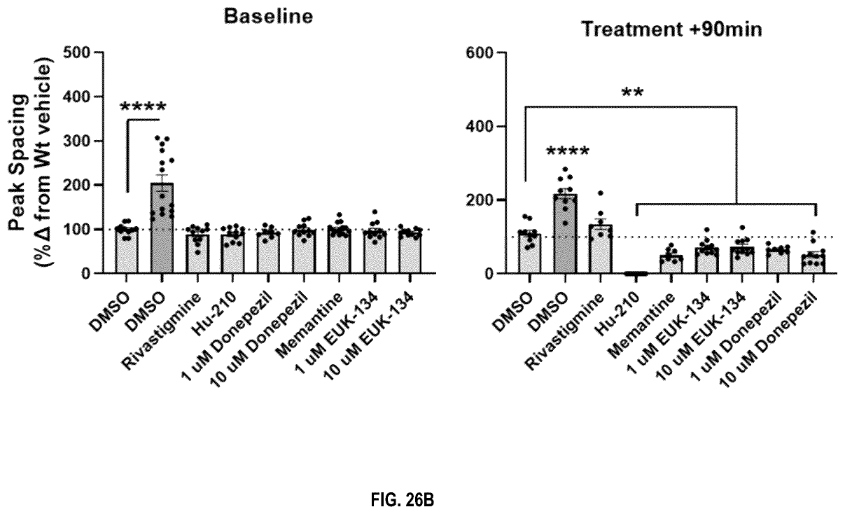

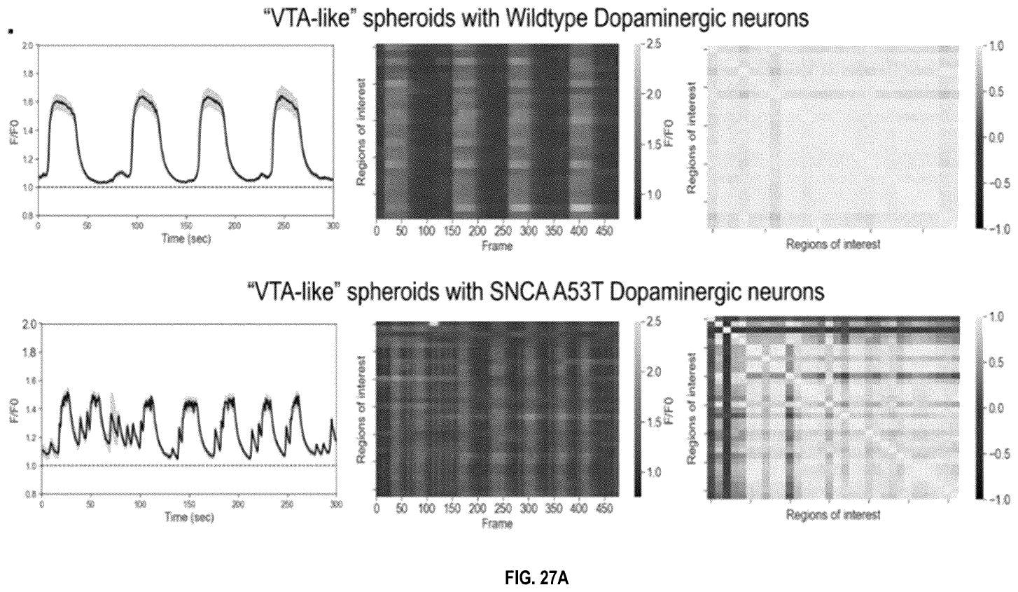

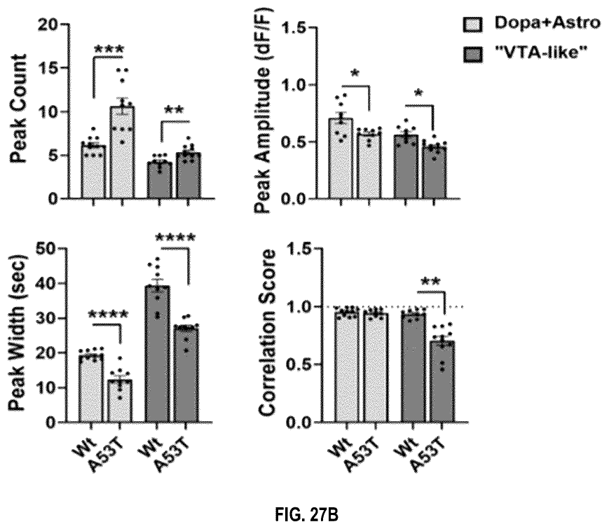

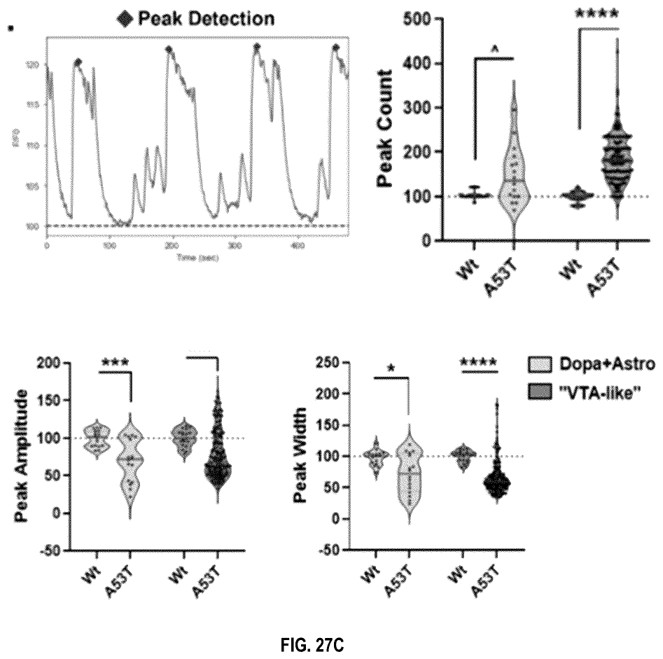

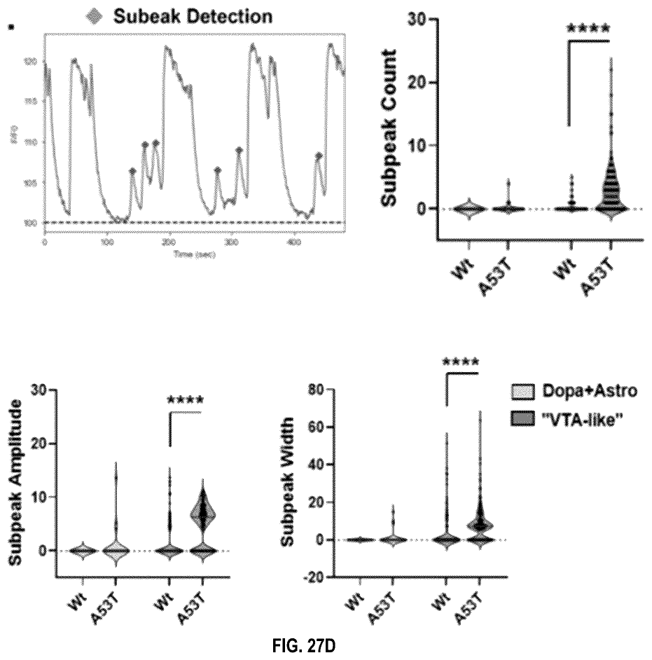

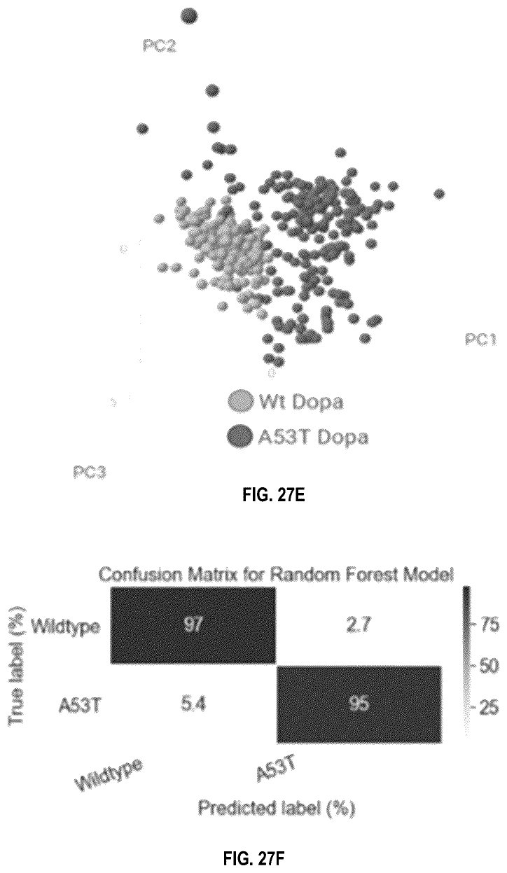

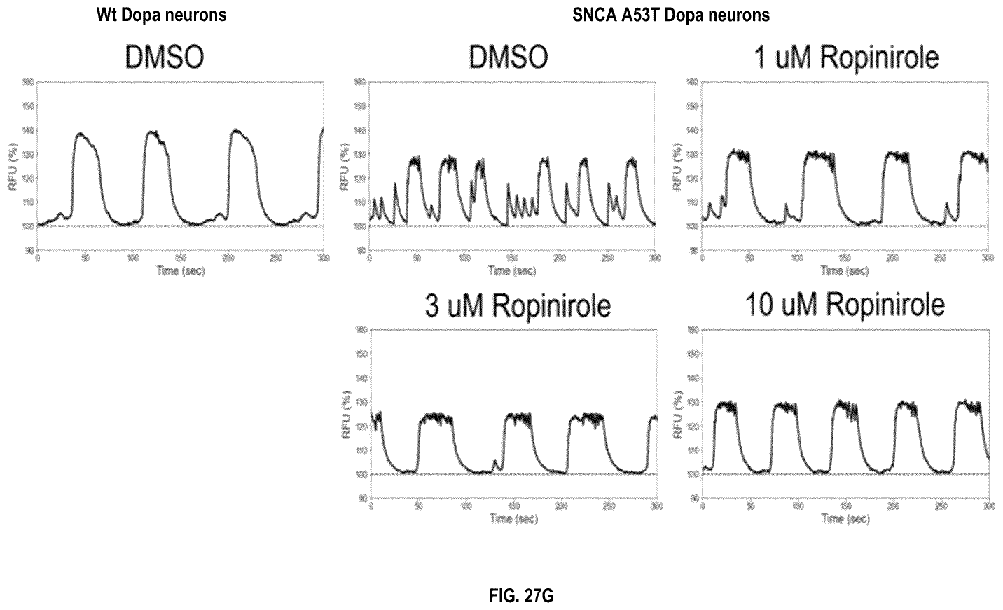

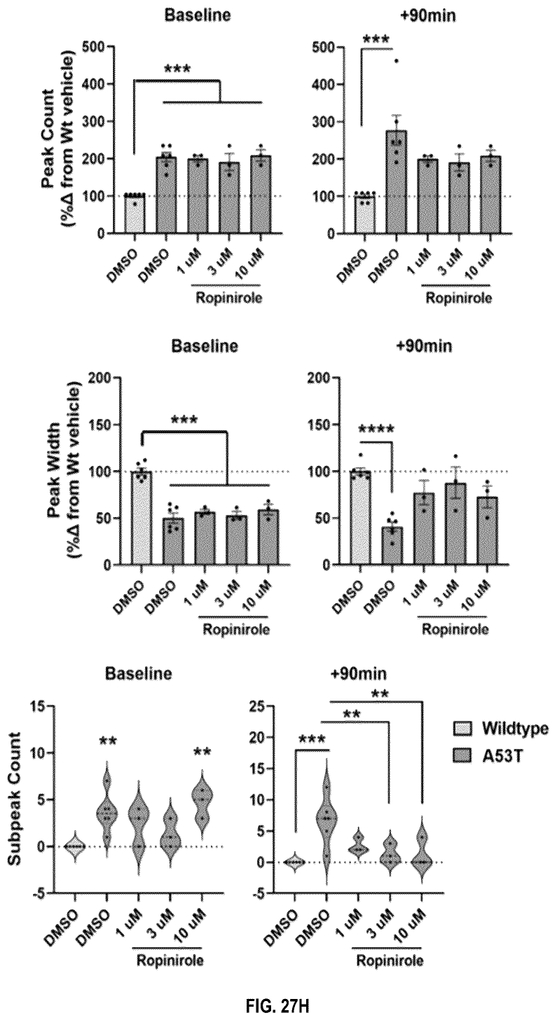

To model Parkinson's Disease (PD), the inventors incorporated dopaminergic neurons expressing mutant A53T alpha-synuclein into spheroids given that it is a common risk factor for non-familial PD. Fernandes et al. (2020) Cell Rep; Petrucci et al. (2016) Parkinsonism Relat Disord; Zambon et al. (2019) Hum Mol Gen. Similar to the AD model described above, A53T dopaminergic neurons were used to make mutant VTA-like spheroids along with control single neuron spheroids (SNSs) containing dopaminergic neurons and consisting of 90% Wt or A53T Dopa neurons and 10% astrocytes. VTA-like spheroids were formed with 65% Wt or A53T dopaminergic neurons plus 5% glutamatergic neurons and 30% GABAergic neurons. To assess whether incorporation of A53T dopaminergic neurons into spheroids impacted cell viability, the 3D CTG assay was used 3-weeks after spheroids were generated. For both spheroid types including SNSs with dopaminergic neurons along with VTA-like spheroids, the genotype of dopaminergic neurons (Wt vs A53T) had no impact of cell viability, suggesting that functional differences between genotypes were not due to differences in cell death caused by the mutant cell line (FIGS. 27A-27I).

After 3-weeks of maintenance, baseline calcium activity was recorded with the PhenixPlus automated confocal microscope from spheroids expressing GCaMP6f, and analysis of peak parameters was performed through the same processes as above with LC_Pro and find_peaks. Here, the inventors surprisingly found functional changes between genotypes that were consistent between single neuron Dopa spheroids and VTA-like spheroids. Specifically, spheroids with A53T Dopa neurons displayed significantly increased peak count and decreased amplitude and peak width (FIG. 27A, 27B). Despite this, the synchronicity of oscillatory patterns between all identified ROIs was unaffected by A53T Dopa neurons in both spheroid types, which is in line with our data showing that disruptions in synchrony are caused by changes in activity of GABA, not Dopa, neurons.

Baseline functional differences between genotypes were also analyzed from FLIPR recordings using a multiparametric approach. Similar to results from PhenixPlus confocal microscopy recordings, spheroids with A53T Dopa neurons displayed significant increases in peak count and decreases in peak width among both the single neuron and VTA-like spheroids (FIG. 27A). While peak amplitude was significantly reduced in A53T spheroids with only Dopa neurons, this effect was not observed in VTA-like spheroids (FIG. 27A). To quantify how predictive the PD spheroid model worked in VTA-like spheroids, PCA was used as a dimension reduction analysis prior to supervised machine learning using the same RFC model described above (FIG. 27C, 27D). Here, baseline FLIPR data from Wt and A53T VTA-like spheroids was accurately labeled in Wild-type (Wt) spheroids 97% of the time and in A53T spheroids 95% of the time, giving an average accuracy score of 96% (FIG. 27D). In summary, the data shows that A53T Dopa neurons alter calcium oscillatory patterns without impacting synchrony, and that these results are consistent across different imaging platforms. Furthermore, using the RFC machine learning algorithm the inventors demonstrated that this functional model for PD is highly predictive.

The Dopamine Agonist, Ropinirole, Reduces A53T Dopa-Mediated Increases in Peak Frequency within VTA-Like Spheroids

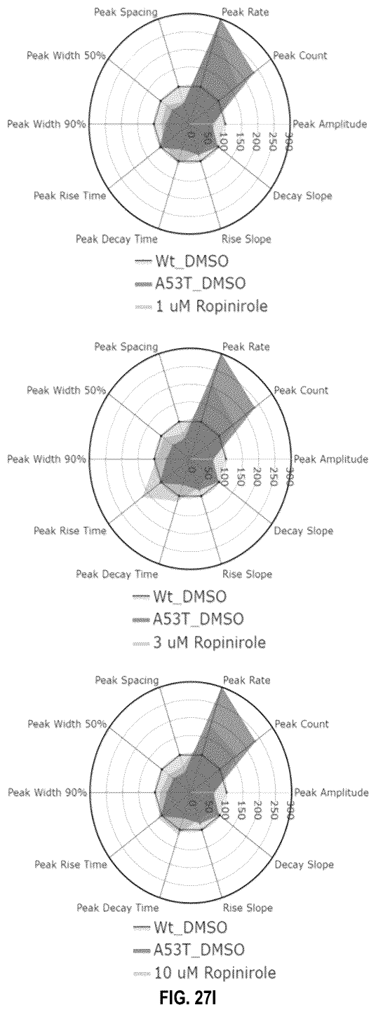

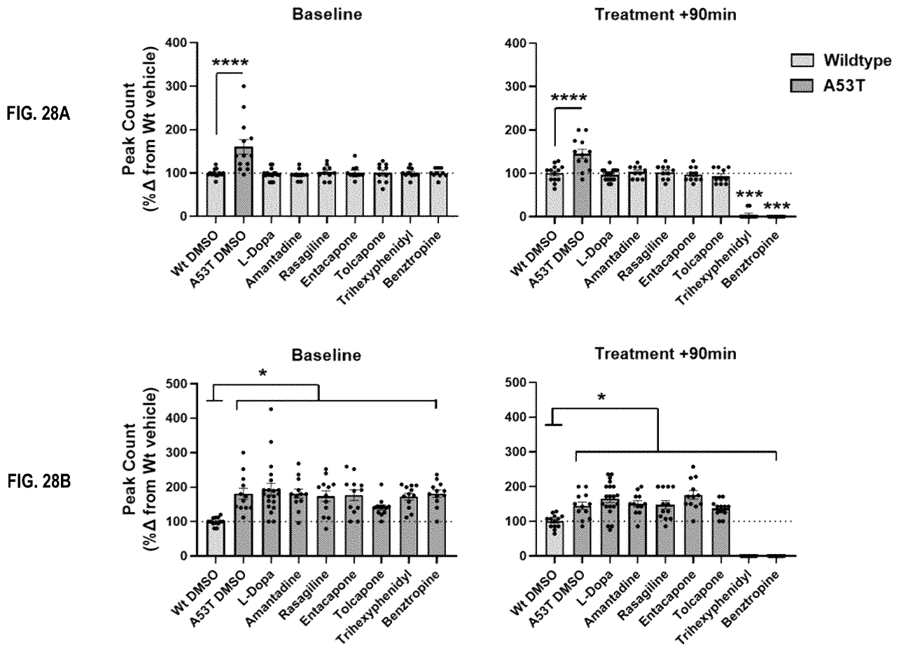

Immediately after the baseline FLIPR recording, spheroids were treated with clinically approved treatments for PD to measure whether they could reverse disease-related deficits in VTA-like spheroids with A53T dopaminergic neurons. These treatments included L-Dopa, used as dopamine replacement therapy, Ropinirole (dopamine agonist), Entacapone and Tolcapone (catechol-O-methyltransferase (COMT) inhibitors), Rasagiline (monoamine oxidase type B (MAO-B) inhibitor), Benztropine (dopamine transporter inhibitor), Trihexyphenidyl (antimuscarinic), and Amantadine (antiviral). Treatment effects were measured with FLIPR recordings 30-, 60-, and 90-min after treatment, and the effects at 90 min were reported. While significant increases in peak count and reductions in peak width were observed across all treatment groups during the baseline recording, only Ropinirole was found to reverse this 90-min after treatment (FIGS. 28A, 28B). Specifically, Ropinirole was tested at 1, 3, and 10 μM and was found to significantly reduce peak count and increase peak width to the level that they were no longer significantly different from Wt DMSO-treated control spheroids (FIGS. 28A, 28B). Furthermore, 3 and 10 μM, but not 1 μM, Ropinirole significantly reduced subpeak count to where they were similar to Wt DMSO-treated controls (FIGS. 28A, 28B). Differences across all peak parameters are displayed as radar plots, where phenotypic profiles for all three concentrations of Ropinirole were tested (FIGS. 28A, 28B). In summary, dopamine agonist, Ropinirole, was able to reverse deficits induced by the incorporate of A53T mutant dopaminergic neurons, showing that our PD model responds to treatments clinically approved to treat PD in humans.

### Chronic DAMGO Pre-Treatment Differentially Affects Brain Region-Specific Neural Spheroids

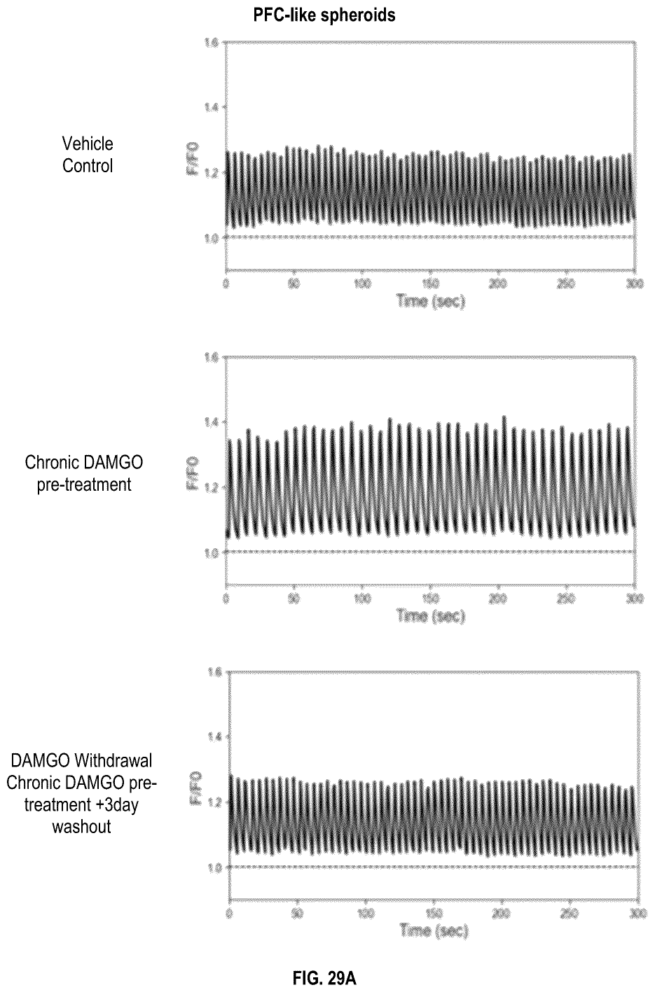

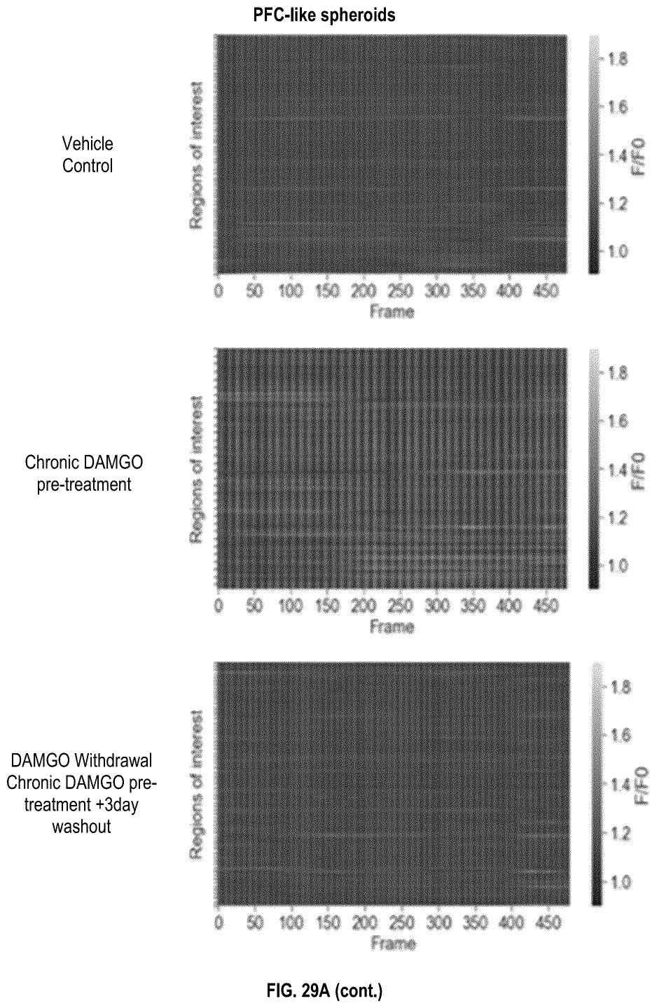

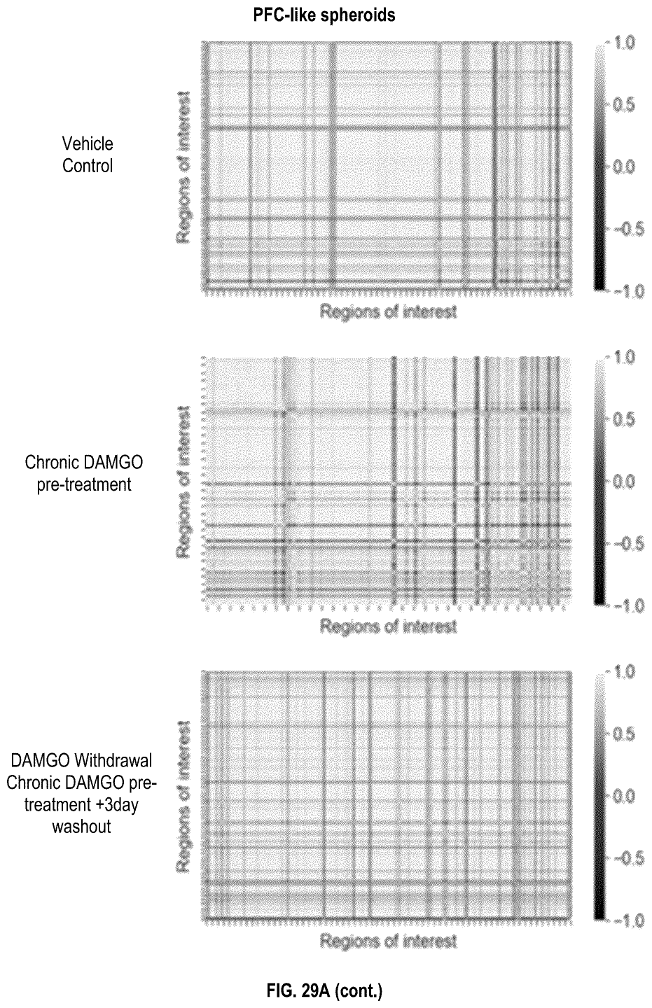

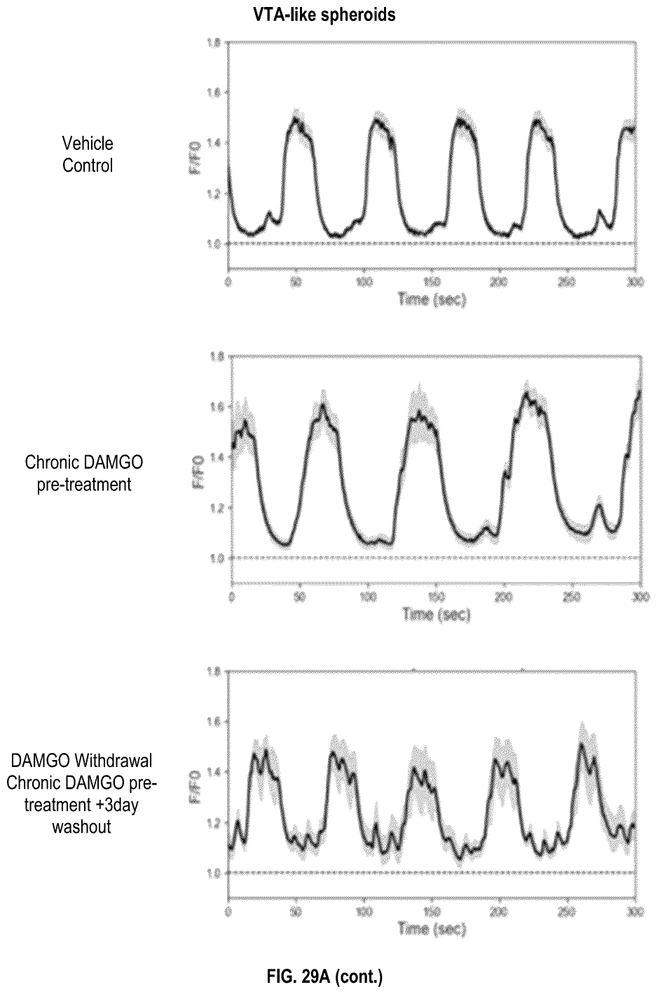

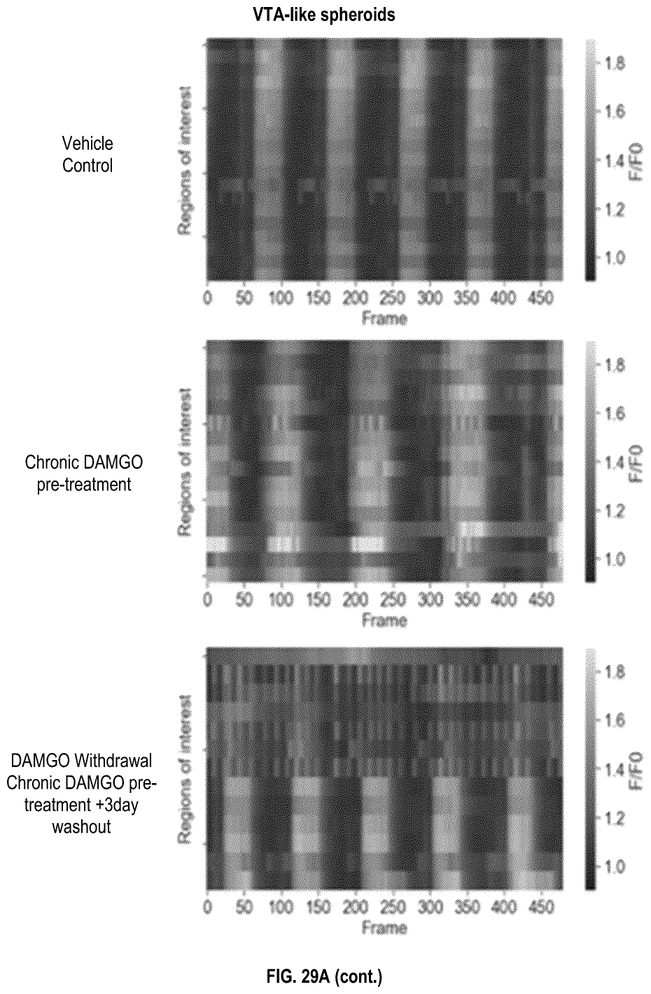

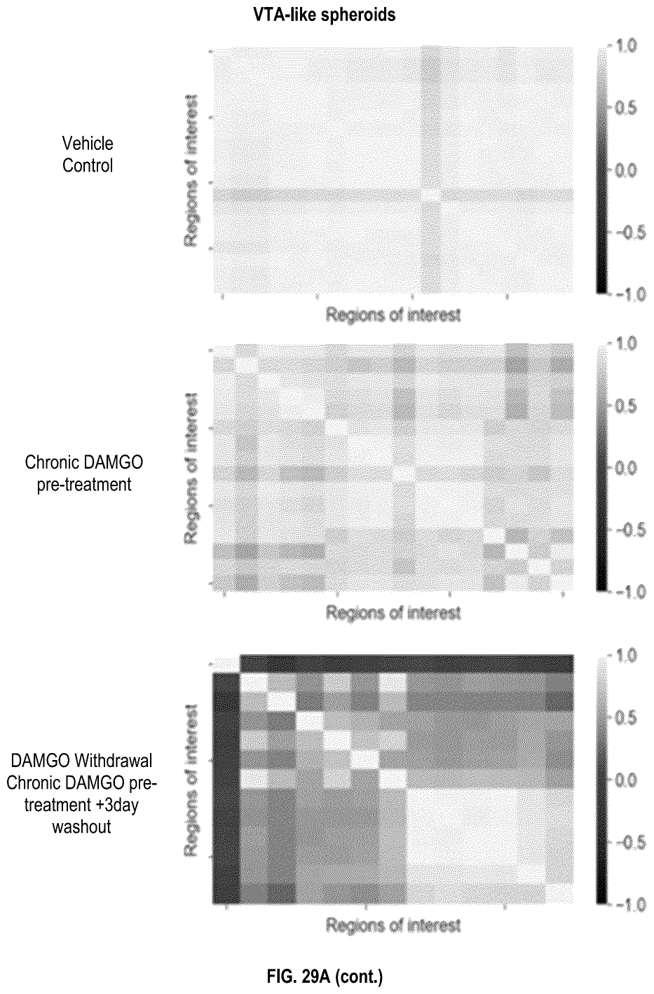

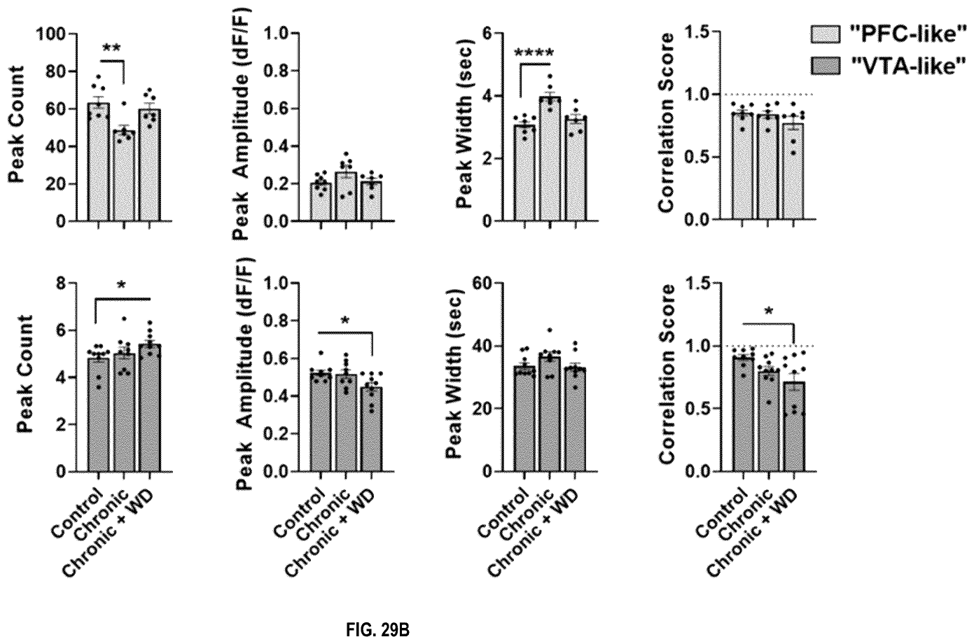

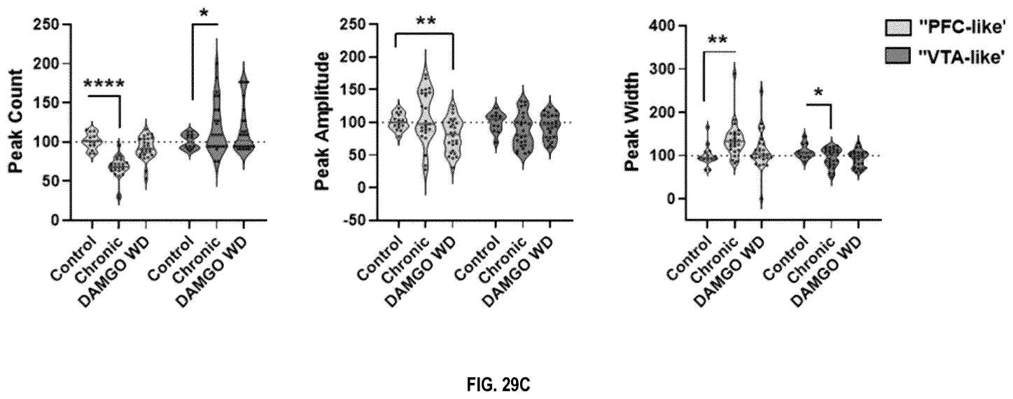

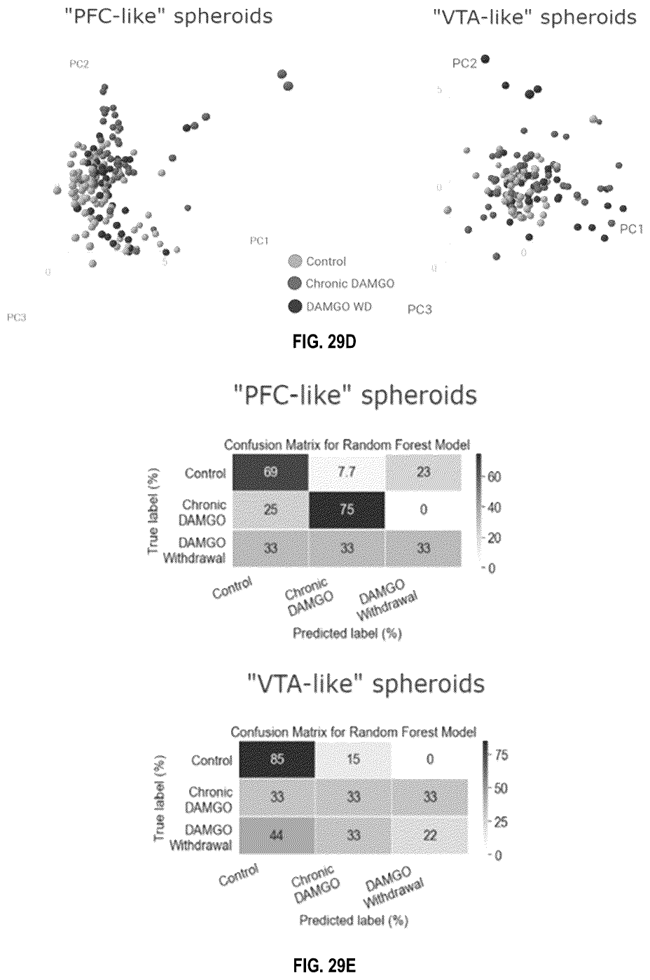

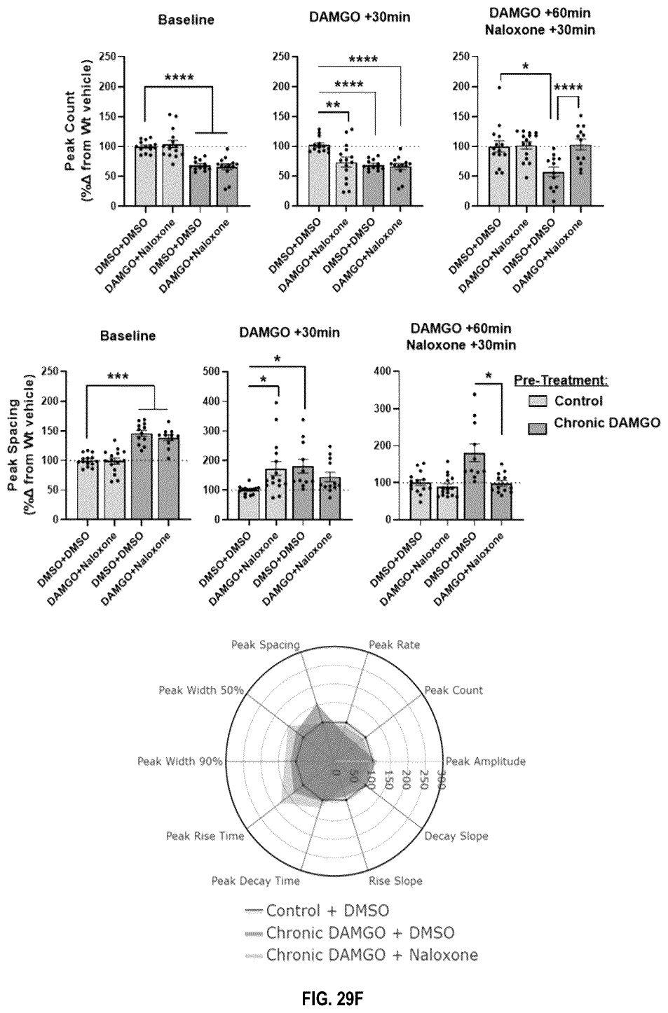

To model opioid use disorder (OUD), the inventors developed a protocol intended to model various facets of addiction including drug intake and withdrawal. To model chronic opioid use, the inventors began adding 20 μM DAMGO, a mu opioid receptor agonist (MOR), to media on days with half media changes, giving a final concentration of 10 μM DAMGO. The inventors began adding DAMGO to the media on day 10 and since the recording was on day 21, a total of five DAMGO exposures occurred for the chronic DAMGO pre-treatment group. To model withdrawal, all conditions were the same except for only four treatments were administered since these spheroids received no drug on the final treatment day. Viability data with 3D CTG showed that spheroid viability was similar between spheroids with no DAMGO pre-treatment, those chronically treated with DAMGO, and those undergoing the DAMGO withdrawal regimen (FIGS. 29A-29F). Further, viability was the same between PFC-like and VTA-like spheroids (FIGS. 29A-29F).

On day 21, baseline calcium imaging recordings were obtained from either a PhenixPlus automated confocal microscope or a FLIPR. Data from the PhenixPlus recordings from spheroids expressing GCaMP6f showed that peak count was reduced by chronic DAMGO treatment in PFC-like spheroids and increased by DAMGO withdrawal in VTA-like spheroids (FIGS. 29A-29F). DAMGO withdrawal reduced peak amplitude in VTA-like but not PFC-like spheroids (FIGS. 29A-29F). Chronic DAMGO increased peak width in PFC-like but not VTA-like spheroids, and DAMGO withdrawal did not impact peak width in either spheroid type (FIGS. 29A-29F). Further, DAMGO withdrawal reduced synchrony in VTA-like spheroids but synchrony was unaffected in PFC-like spheroids (FIGS. 29A-29F).

Baseline calcium activity for the OUD model was also collected with a FLIPR from spheroids incubating in Cal6 dye, and this data showed that peak count was reduced by chronic DAMGO in PFC-like spheroids but that chronic DAMGO treatment increased peak count in VTA-like spheroids (FIGS. 29A-29F). Peak amplitude was reduced by DAMGO withdrawal in PFC-like spheroids but unaffected in VTA-like spheroids, however peak width was increased by chronic DAMGO in PFC-like spheroids but reduced in VTA-like spheroids (FIGS. 29A-29F). Multiparametric differences between pre-treatment groups are represented as radar plots for both spheroid types (FIGS. 29A-29F). Similar to our AD and PD models, the inventors used PCA followed by the RFC model to quantify how predictive the calcium activity phenotypes are for each disease line (FIGS. 29A-29F). In PFC-like spheroids, accuracy for predicting phenotypes of control spheroids previously un-treated with DAMGO was only 50% with the other 50% of errors occurring in the DAMGO withdrawal group (FIGS. 29A-29F). Accuracy for predicting labels of chronic DAMGO was only 43%, though 43% of errors occurred in the DAMGO withdrawal group and 14% in control spheroids, while accuracy for the DAMGO withdrawal group was 50% with 33% of errors occurring in the chronic DAMGO group and 17% in control spheroids (FIGS. 29A-29F). For VTA-like spheroids, the RFC model was 67% accurate at predicting the label of control spheroids, with 33% of errors occurring in the chronic DAMGO group while it was 80% accurate at predicting labels of the chronic DAMGO group, with 20% of errors occurred in the control group (FIGS. 29A-29F). For the DAMGO withdrawal group in VTA-like spheroids, 57% of labels were accurate while 29% labeled data as chronic DAMGO-treated and 14% labeled the spheroid as a control (FIGS. 29A-29F). Taken together, this data shows that while chronic DAMGO pre-treatment impacted measures of both oscillatory peak parameters and synchrony, the model is not as predictive as the AD or PD model.

After the baseline FLIPR recording, spheroids were treated with 10 μM DAMGO one final time, and activity was recorded 30-min later, followed immediately by naloxone, an MOR antagonist used to reverse opioid overdoses in humans, via a 384-well pin tool. Here, both control and chronic DAMGO-treated spheroids were either treated with DMSO prior to each recording (DMSO+DMSO) or DAMGO followed by naloxone (DAMGO+Naloxone). At baseline, peak count was significantly decreased and peak spacing was significantly increased in PFC-like spheroids for both the DMSO+DMSO group and the DAMGO+Naloxone group (FIGS. 29A-29F, panels titled Baseline). 30-min after treatment with either DMSO or DAMGO, a significant reduction in peak count was observed in control spheroids acutely treated with DAMGO while chronic DAMGO-treated spheroids treated with either DMSO or DAMGO showed significant reductions in peak count (FIGS. 29A-29F, panels titled DAMGO+30 min). For peak spacing, acute DAMGO treatment in control spheroids increased peak spacing, and peak spacing remained significantly increased in chronic DAMGO-treated spheroids that were treated with DMSO, but not DAMGO (FIGS. 29A-29F, panels titled DAMGO+30 min). Spheroids were treated with either DMSO or Naloxone and a recording was obtained 30-min later. Here, the inventors found that naloxone reversed deficits in peak count and spacing in acute DAMGO-treated control spheroids since these peak parameters were no longer significantly different from spheroids treated with DMSO (FIGS. 29A-29F, panels titled DAMGO+30 min, Naloxone+60 min). Furthermore, while chronic DAMGO spheroids treated with DMSO still showed significant differences in peak count and spacing, those treated with naloxone no longer showed significant differences in these peak parameters compared to DMSO-treated controls, suggesting naloxone reversed these deficits (FIGS. 29A-29F, panels titled DAMGO+30 min, Naloxone+60 min). Changes across all 10 peak parameters measured during the 60-min recording are displayed via a radar plot in FIG. 29F. Here the inventors show that opioid treatment with DAMGO impacts activity in spheroids acutely treated along with those receiving treatment after chronic DAMGO pre-treatment, and that blocking MORs with naloxone is able to reverse these changes.

Functional Assembloids Made from Conjoining VTA- and PFC-Like Spheroids can be Used to Model Neural Circuitry

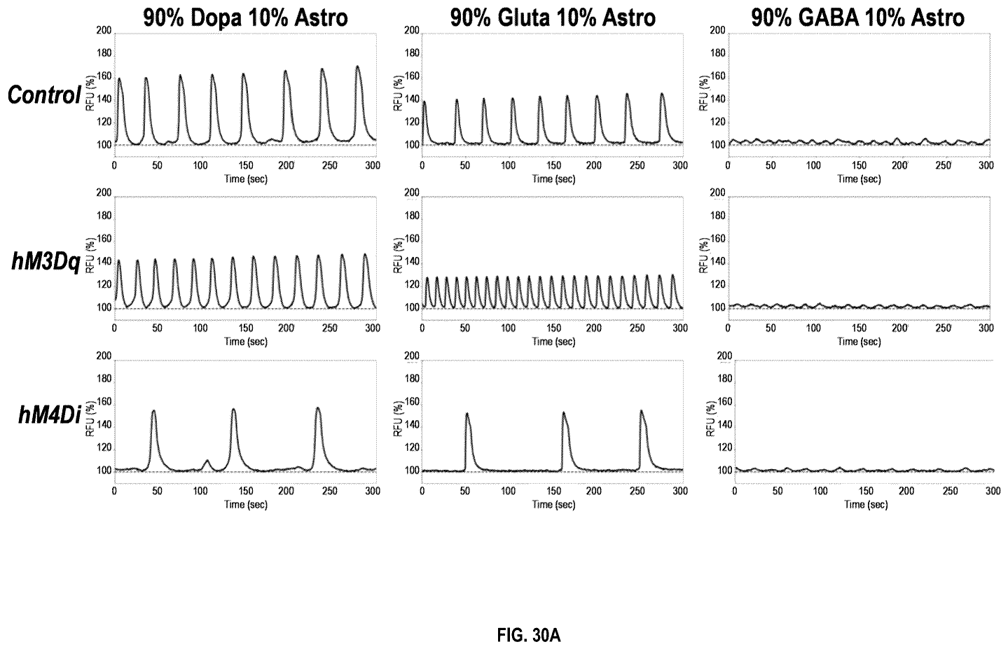

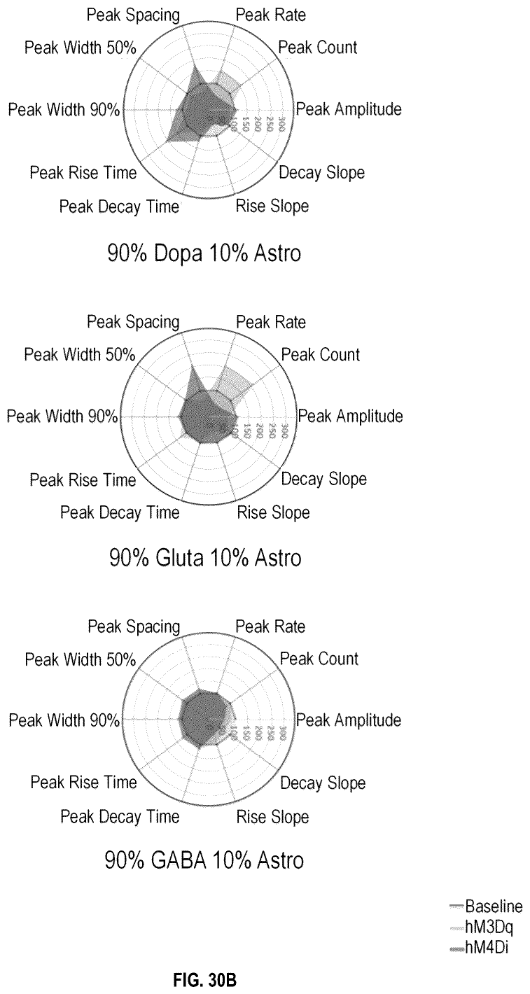

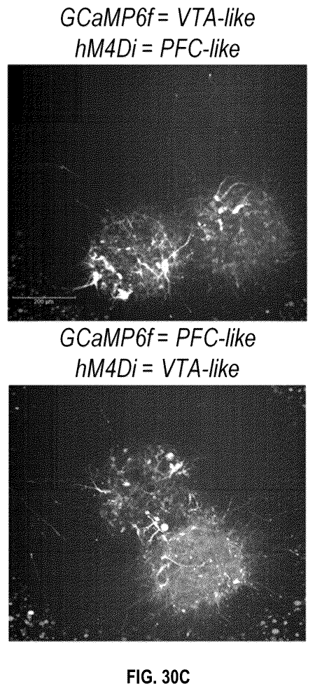

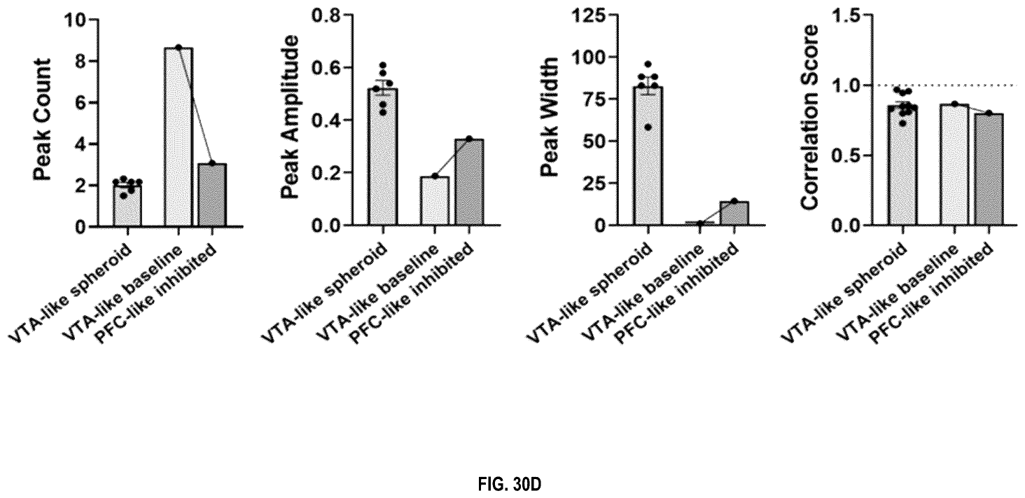

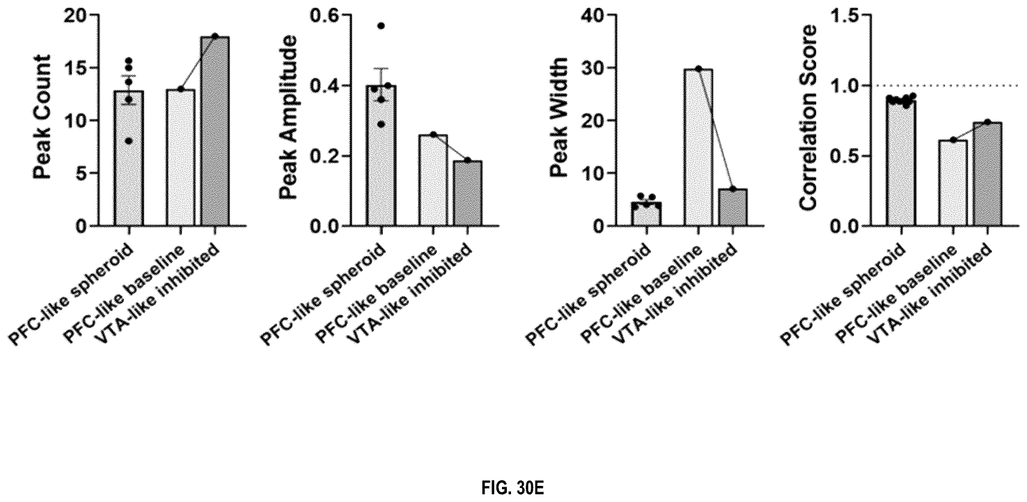

Prior to making assembloids, the inventors employed a proof-of-concept experiment using a chemogenetic approach to examine whether calcium activity could be both enhanced and inhibited (FIGS. 30A-30E). On day 7, the inventors transduced spheroids with retrograde AAV viruses fused with an mCherry fluorophore to avoid cross excitation with Cal6 dye (550-670 nm emission vs 515-575 nm emission, respectively), which inserted either stimulatory (hM3Dq) or inhibitory (hM4Di) designer receptors that are exclusively activated by designer drugs (DREADDs) tagged. Clozapine-N-oxide (CNO) was used at a 1 uM concentration to activate the DREADDs viruses, and representative time series traces show the effect 30-min after treatment with FLIPR recordings (FIGS. 30A-30E). Radar plots were generated showing the effect of DREADDs across all ten peak parameters with low CV values (FIGS. 30A-30E), and mean values for each parameter are reported. For spheroids with Dopa or Gluta composition, chemogenetic activation increased measures of peak frequency such as peak count and rate while reducing parameters such as peak spacing and peak width, while the opposite was observed for these spheroids subjected to chemogenetic inhibition (FIGS. 30A-30E).

Given that the cell type composition of these spheroids can be altered to model various brain regions, the inventors tested neural circuit-specific projections could be modeled by creating assembloids via the fusing of two spheroids together. One week prior to recording activity, assembloids were created by combining a PFC-like and VTA-like spheroid into one well and casting in collagen. Specifically, the inventors paired brain region-specific spheroids together such that one expressed GCaMP6f and the other expressed the inhibitory DREADDs virus, hM4Di (FIGS. 30A-30E). The inventors first recorded from assembloids where the VTA-like component of the assembloid expressed GCaMP6f and the PFC-like component expressed hM4Di (FIGS. 30A-30E). Both a baseline recording along with a recording 60-min after treatment with 1 uM CNO to activate hM4Di were collected (FIGS. 30A-30E). Here, at baseline, peak count was increased when recording from the VTA-like component within the assembloid, but that CNO treatment reduced this effect to the level of VTA-like spheroids (FIGS. 30A-30E). Furthermore, baseline peak amplitude and width were reduced in the VTA-like component of the assembloid and inhibition of the PFC-like component of the assembloid increased this, though not to the level of VTA-like spheroids (FIGS. 30D, 30E). Lastly, correlation score was unaffected, suggesting that inhibiting part of the circuit did not impact synchronous activity.

The inventors also recorded from an assembloid where the PFC-like component expressed GCaMP6f and the VTA-like component expressed hM4Di (FIGS. 30F, 30G). The inventors observed similar baseline peak count in the PFC-like component of the assembloid as the inventors observed in PFC-like spheroids, though inhibiting the VTA-like component of the assembloid further increased peak count (FIGS. 30F, 30G). Additionally, baseline peak amplitude was reduced when compared to PFC-like spheroids, and the inventors found that inhibiting the VTA-like component of the assembloid further reduced this (FIGS. 30F, 30G). However, the inventors observed increased peak width at baseline in the PFC-like component of the assembloid compared to PFC-like spheroids, and inhibiting the VTA-like component of the assembloid brought this level back to that of PFC-like spheroids (FIGS. 30F, 30G). Lastly, the inventors saw that synchrony was reduced in the PFC-like component of the assembloid compared to PFC-like spheroids, and that inhibiting the VTA-like component of the assembloid slightly increased this (FIGS. 30F, 30G). Taken together, this data shows that brain region-specific neural circuits can be modeled with these spheroids, and that calcium activity phenotypes can be modulated by manipulating different components of the assembloid.

### Discussion

While currently available 3D neural spheroid models that differentiate neural stem cells into neuronal and glial subtypes in culture show more consistent and robust activity compared to 2D models, they lack control over proportions of cell types within them and primarily model cortical brain regions. To improve upon currently existing methods, the inventors created brain region-specific neural spheroids such that the neuronal cell type composition reflected what is represented in the human brain. The inventors focused on two brain regions specifically, and created spheroids with neuronal subtype distributions modeling the human prefrontal cortex (PFC-like spheroids) and ventral tegmental area (VTA-like spheroids) given the role these two regions play in neurological diseases including opioid use disorder (OUD), Parkinson's Disease, and Alzheimer's Disease.

Current in vitro neural models range from two-dimensional (2D) monolayer cellular assay systems to 3D brain organoid models. 2D cultured cells are robust in the sense that they can be seeded in multiwell plates for high-throughput (HT) study designs but display low functional reproducibility well-to-well and do not adequately model in vivo neurophysiology. Studies have shown that 2D monolayers of neural cells show shorter neurite growth and reduced gene expression for markers of neuronal function, extracellular matrix, and cytoskeleton compared to 3D models. Chandrasekaran et al. (2017) Stem Cell Res. 25: 139-151; Smith et al. 2017. Given that synapses are formed through the growth of pre- and postsynaptic densities whereby neurotransmitters and ions are released to promote neuronal communication in the form of action potentials, differences in activity between tissue models can occur when the morphology occurs in such a way that impedes synaptic growth (Hodgkin and Huxley, 1939; Hausser, 2000; Schuetze, 1983). In line with this, studies have observed reduced neuronal activity in 2D models that is less functionally reproducible than in 3D models (Smith et al., 2017; Woodruff et al., 2020).

In recent years, the development of three-dimensional (3D) iPSC-derived brain organoids has provided an important step towards bridging the gap for more predictable and translatable in vitro models of neurological diseases. With the development of forebrain, midbrain, and hindbrain organoids it has been shown that organoids can contain some degree of cellular organization, mimicking human brain anatomy, and therefore are more physiologically relevant than 2D cellular models. Furthermore, neural circuit modeling has been established via the fusion of two different organoid types to create functional assembloids, which could help establish models for neurological diseases impacting specific neural circuits such as Parkinson's Disease (PD) and substance use disorder. While organoid models have made significant inroads as complex 3D neural models, their complexity hinders their ability to be implemented in high-throughput drug screening (HTS) assay platforms. For instance, organoids can suffer from batch-to-batch variation in both size and cell composition heterogeneity, limited differentiation of neuronal cell types, and lengthy differentiation and maturation times. As such, there is a need for tissue models that can balance the robustness of 2D cellular models with the complexity of 3D organoids.

This spontaneous, synchronous activity arises from local field potentials (LFPs) generated from the summation of spontaneously generated action potentials from networks of neurons, and intracellular calcium oscillations have been shown to be highly correlated with the electrophysiological properties of neurons. The inventors recorded calcium activity using a calcium dye (Cal6) on two platforms that differed in high-throughput capability and amount of data generated. Image-based single-well recordings were obtained with an automated confocal microscope to measure fluctuations in calcium fluorescence in individual cells within a spheroid, and a fluorescent imaging plate reader (FLIPR) was used to record fluctuations in population spheroid activity simultaneously across all wells on the 384-well plate. Similar phenotypic profiles were observed between the two recording platforms, and while the automated confocal recordings were useful for quantifying synchrony within spheroids, the FLIPR is amenable for HTS studies.

All references cited in this specification are herein incorporated by reference as though each reference was specifically and individually indicated to be incorporated by reference. The citation of any reference is for its disclosure prior to the filing date and should not be construed as an admission that the present disclosure is not entitled to antedate such reference by virtue of prior invention.

It will be understood that each of the elements described above, or two or more together may also find a useful application in other types of methods differing from the type described above. Without further analysis, the foregoing will so fully reveal the gist of the present disclosure that others can, by applying current knowledge, readily adapt it for various applications without omitting features that, from the standpoint of prior art, fairly constitute essential characteristics of the generic or specific aspects of this disclosure set forth in the appended claims. The foregoing embodiments are presented by way of example only; the scope of the present disclosure is to be limited only by the following claims.

1-202. (canceled)

203. A method of making an isolated neural spheroid, the method comprising:
admixing differentiated neurons, the differentiated neurons comprising two or more neuronal types selected from GABAergic neurons, glutamatergic neurons, dopaminergic neurons, cholinergic neurons, and serotonergic neurons; and
culturing the admixed differentiated neurons to form the isolated neural spheroid.

204. The method of claim 203, wherein the isolated neural spheroid exhibits one or more properties of cells from one or more defined brain regions selected from the ventral tegmental area (VTA), prefrontal cortex (PFC), nucleus accumbens, amygdala, hippocampus, somatomotor cortex, somatosensory cortex, parietal lobe, occipital lobe, cerebellum, and temporal lobe.

205. The method of claim 203, wherein the admixing further comprises admixing differentiated glial cells with the differentiated neurons, the differentiated glial cells comprising at least one glial cell type selected from astrocytes, microglia, and oligodendrocytes.

206. The method of claim 205, wherein the differentiated neurons, or the differentiated glial cells, or both are human cells.

207. The method of claim 205, wherein the differentiated neurons, or the differentiated glial cells, or both are derived from induced pluripotent stem cells (hiPSCs).

208. The method of claim 205, wherein:
the admixed differentiated neurons comprise GABAergic neurons and glutamatergic neurons;
the admixed differentiated glial cells comprise astrocytes; and
the isolated neural spheroid exhibits one or more properties of cells from the prefrontal cortex (PFC).

209. The method of claim 208, wherein the admixed differentiated neurons and differentiated glial cells comprise GABAergic neurons, glutamatergic neurons, and astrocytes in a ratio of 7 to 7.5 glutamatergic neurons: 2.5 to 3 GABAergic neurons: 1 astrocyte.

210. The method of claim 208, wherein the admixed differentiated neurons and differentiated glial cells comprise about 60% to about 80% glutamatergic neurons and from about 20% to about 40% GABAergic neurons by percentage of neurons, and from about 5% to about 15% astrocytes by total percentage of admixed cell number.

211. The method of claim 205, wherein:
the admixed differentiated neurons comprise GABAergic neurons, glutamatergic neurons, and dopaminergic neurons;
the admixed differentiated glial cells comprise astrocytes; and
the isolated neural spheroid exhibits one or more properties of cells from the ventral tegmental area (VTA).

212. The method of claim 211, wherein the admixed differentiated neurons and differentiated glial cells comprise GABAergic neurons, glutamatergic neurons, dopaminergic neurons, and astrocytes in a ratio of 3 to 3.5 GABAergic neurons: 0.5 glutamatergic neurons: 6.0 to 6.5 dopaminergic neurons: 1 astrocyte.

213. The method of claim 211, wherein the admixed differentiated neurons and differentiated glial cells comprise from about 55% to about 75% dopaminergic neurons, from about 2.5% to about 7.5% glutamatergic neurons, from about 20% to about 40% GABAergic neurons by percentage of neurons, and from about 5% to about 15% astrocytes by total percentage of admixed cell number.

214. The method of claim 203, wherein the admixing further comprises admixing endothelial cells, or pericytes, or both with the differentiated neurons.

215. The method of claim 203, wherein cells of the isolated neural spheroid comprise transfected cells, the cells transfected using a viral construct comprising a transgene.

216. The method of claim 203, further comprising suspending at least two isolated neural spheroids in a matrix to form an assembloid.

217. The method of claim 203, further comprising measuring one or more properties, the one or more properties comprising an electrophysiological property, a calcium activity profile, neurotransmitter release, neurotransmitter uptake, cell death, or synchrony, or any combination thereof, in the presence and absence of an agent.

218. The method of claim 203, further comprising:
exposing the isolated neural spheroid to a test compound;
measuring an activity of the isolated neural spheroid and collecting activity data, wherein the activity measured is cell death, calcium activity, neurotransmitter release, or neurotransmitter uptake, or a combination thereof; and
comparing the activity measured with an activity of an isolated neural spheroid not exposed to the test compound.

219. The method of claim 203, further comprising:
chronically treating, or acutely treating, or both the isolated neural spheroid with a mu opioid receptor (MOR) agonist;
exposing the isolated neural spheroid to a test compound;
measuring an activity of the isolated neural spheroid; and
comparing the measured activity against the activity measured in a control isolated neural spheroid not treated with the mu opioid receptor (MOR) agonist.

220. The method of claim 208, further comprising:
exposing the neural spheroid to a test compound, wherein cells of the neural spheroid have been transfected with a APOE4 transgene;
measuring an activity of the neural spheroid; and
comparing the measured activity against the activity measured in a control isolated neural spheroid, comprising cells that have been transfected with the APOE4 transgene, not treated with the test compound, or a control isolated neural spheroid not transfected with the APOE4 transgene, or both.

221. The method of claim 211, further comprising:
exposing the neural spheroid to a test compound wherein cells of the neural spheroid have been transfected with a A53T mutant alpha-synuclein transgene;
measuring an activity of the neural spheroid; and
comparing the measured activity against the activity measured in a control isolated neural spheroid, comprising cells that have been transfected with the A53T mutant alpha-synuclein transgene, not treated with the test compound, or a control isolated neural spheroid not transfected with the A53T mutant alpha-synuclein transgene, or both.

222. The isolated neural spheroid produced by the method of claim 203.
